SEQUENCE LISTING (212 sequences)

SEQ ID NO: 1 - protein, 117 aa, Artificial Sequence
  Synthetic construct
EVQLQESGPSLVKPSQTLSLTCSVTGDSITSGYWNWIRKFPGNKLEYMGYISYSGSTYYNLSLRSRISITRDTSKNQYYLQLNSVTTEDTATYYCALITTTTYAMDYWGQGTSVTVS

SEQ ID NO: 2 - protein, 111 aa, Artificial Sequence
  Synthetic construct
NIVLTQSPVSLAVSLGQRATISCRASESVDGYGNSFLHWFQQKPGQPPKLLIYLASNLNSGVPARFSGSGSRTDFTLTIDPVEADDAATYYCQQNNVDPWTFGGGTKLEIK

SEQ ID NO: 3 - protein, 117 aa, Artificial Sequence
  Synthetic construct
QVQLQESGPGLVKPSETLSLTCTVSGDSISSGYWNWIRQPPGRALEWIGYISYSGSTYYNLSLRSRVTISVDTSKNQFSLRLSSVTAADTAMYYCARITTTTYAMDYWGQGTTVTVS

SEQ ID NO: 4 - protein, 111 aa, Artificial Sequence
  Synthetic construct
DIVLTQSPDSLSVSLGERVTVNCRASESVDGYGNSFLHWFQQNPGQPPKLLIYLASNLNSGVPARFMGSGSGTEFSLTISSLQAEDVAVYYCQQNNVDPWTFGQGTKLEIN

SEQ ID NO: 5 - protein, 111 aa, Artificial Sequence
  Synthetic construct
DIVLTQSPDSLSVSLGERVTVNCRASESVDGYGNSFLHWFQQNPGQPPKLLIYLASNLNSGVPARFMGSGSRTEFSLTISSLQAEDVAVYYCQQNNVDPWTFGQGTKLEIK

SEQ ID NO: 6 - protein, 111 aa, Artificial Sequence
  Synthetic construct
EIVLTQSPSSLSASVGDRVTITCRASESVDGYGNSFLHWYQQKPGKAPKLLIYLASNLNSGVPSRFSGSGSGTDFTLTISSLQPEDFATYYCQQNNVDPWTFGQGTKLEIK

SEQ ID NO: 7 - protein, 111 aa, Artificial Sequence
  Synthetic construct
EIVLTQSPSSLSASVGDRVTITCRASESVDGYGNSFLHWFQQKPGKAPKLLIYLASNLNSGVPSRFSGSGSRTDFTLTISSLQPEDFATYYCQQNNVDPWTFGQGTKLEIK

SEQ ID NO: 8 - protein, 117 aa, Artificial Sequence
  Synthetic construct
EIVLTQSPLSLPVTLGQPASISCRASESVDGYGNSFLHWFQQRPGQSPRLLIYLASNLNSGVPDRFSGSGSRTDFTLKISRVEAEDVGVYYCQQNNVDPWTFGGGTKVEIKREWIPR

SEQ ID NO: 9 - protein, 111 aa, Artificial Sequence
  Synthetic construct
DIVMTQSPDSLAVSLGERATINCRASESVDGYGNSFLHWYQQKPGQPPKLLIYLASNLNSGVPDRFSGSGSGTDFTLTISSLQAEDVAVYYCQQNNVDPWTFGQGTKLEIK

SEQ ID NO: 10 - protein, 117 aa, Artificial Sequence
  Synthetic construct
QVQLQESGPGLVKPSETLSLTCTVSGDSITSGYWNWIRQPPGRALEYMGYISYSGSTYYNLSLRSRITISRDTSKNQYSLRLSSVTAADTAMYYCALITTTTYAMDYWGQGTTVTVS

SEQ ID NO: 11 - protein, 117 aa, Artificial Sequence
  Synthetic construct
QVQLQESGPGLVKPSETLSLTCTVSGDSITSGYWNWIRKPPGRALEYMGYISYSGSTYYNLSLRSRITISRDTSKNQYSLRLSSVTAADTAMYYCALITTTTYAMDYWGQGTTVTVS

SEQ ID NO: 12 - protein, 118 aa, Artificial Sequence
  Synthetic construct
QVQLQESGPGLVKPSETLSLTCTVSGDSISSGYWNWIRQPPGRALEYMGYISYSGSTYYNLSLRSRITISRDTSKNQYSLRLSSVTAADTAMYYCALITTTTYAMDYWGQGTTVTVSS

SEQ ID NO: 13 - protein, 118 aa, Artificial Sequence
  Synthetic construct
QVQLQESGPGLVKPSETLSLTCTVSGDSITSGYWNWIRQPPGRALEWMGYISYSGSTYYNLSLRSRITISRDTSKNQYSLRLSSVTAADTAMYYCALITTTTYAMDYWGQGTTVTVSS

SEQ ID NO: 14 - protein, 117 aa, Artificial Sequence
  Synthetic construct
QVQLQESGPGLVKPSETLSLTCTVSGDSITSGYWNWIRQPPGRALEYIGYISYSGSTYYNLSLRSRITISRDTSKNQYSLRLSSVTAADTAMYYCALITTTTYAMDYWGQGTTVTVS

SEQ ID NO: 15 - protein, 117 aa, Artificial Sequence
  Synthetic construct
QVQLQESGPGLVKPSETLSLTCTVSGDSITSGYWNWIRQPPGRALEYMGYISYSGSTYYNLSLRSRVTISRDTSKNQYSLRLSSVTAADTAMYYCALITTTTYAMDYWGQGTTVTVS

SEQ ID NO: 16 - protein, 116 aa, Artificial Sequence
  Synthetic construct
QVQLQESGPGLVKPSETLSLTCTVSGDSITSGYWNWIRQPPGRALEYMGYISYSGSTYYLSLRSRITISVDTSKNQYSLRLSSVTAADTAMYYCALITTTTYAMDYWGQGTTVTVS

SEQ ID NO: 17 - protein, 117 aa, Artificial Sequence
  Synthetic construct
QVQLQESGPGLVKPSETLSLTCTVSGDSITSGYWNWIRQPPGRALEYMGYISYSGSTYYNLSLRSRITISRDTSKNQFSLRLSSVTAADTAMYYCALITTTTYAMDYWGQGTTVTVS

SEQ ID NO: 18 - protein, 117 aa, Artificial Sequence
  Synthetic construct
QVQLQESGPGLVKPSETLSLTCTVSGDSITSGYWNWIRQPPGRALEYMGYISYSGSTYYNLSLRSRITISRDTSKNQYSLRLSSVTAADTAMYYCARITTTTYAMDYWGQGTTVTVS

SEQ ID NO: 19 - protein, 111 aa, Artificial Sequence
  Synthetic construct
EIVLTQSPSSLSASVGDRVTITCRASESVDGYGNSFLHWFQQKPGKAPKLLIYLASNLNSGVPSRFSGSGSGTDFTLTISSLQPEDFATYYCQQNNVDPWTFGQGTKLEIK

SEQ ID NO: 20 - protein, 111 aa, Artificial Sequence
  Synthetic construct
EIVLTQSPSSLSASVGDRVTITCRASESVDGYGNSFLHWYQQKPGKAPKLLIYLASNLNSGVPSRFSGSGSRTDFTLTISSLQPEDFATYYCQQNNVDPWTFGQGTKLEIK

SEQ ID NO: 21 - DNA, 351 bp, Artificial Sequence
  Synthetic construct
gaggtgcagcttcaggagtcaggacctagcctcgtgaaaccttctcagactctgtccctcacctgttctgtcactggcgactccatcaccagtggttactggaactggatccggaaattcccagggaataaacttgagtacatgggatacataagttacagtggtagcacttactacaatctatctctcagaagtcgcatctccatcactcgagacacatccaagaatcagtactacctgcagttgaattctgtgactactgaggacacagccacatattactgtgcgctcattactacgactacctatgctatggactactggggtcaaggaacctcagtcaccgtctcc

SEQ ID NO: 22 - DNA, 333 bp, Artificial Sequence
  Synthetic construct
aacattgtgctgacccaatctccagtttctttggctgtgtctctggggcagagggccaccatttcctgcagagccagtgaaagtgttgatggttatggcaatagttttctgcactggttccagcagaaaccaggacagccacccaaactcctcatctatcttgcatccaacctaaactctggggtccctgccaggttcagtggcagtgggtctaggacagacttcaccctcaccattgatcctgtggaggctgatgatgctgcaacctattactgtcagcaaaataatgtggacccgtggacgttcggtggaggcaccaagctggaaatcaaa

SEQ ID NO: 23 - DNA, 351 bp, Artificial Sequence
  Synthetic construct
caagtgcagctgcaggagtcgggaccaggactggtgaagccttcggagaccctgtccctcacctgcactgtctctggtgactccatcagtagtggttactggaactggatccggcagcccccagggagggcactggagtggataggatacataagttacagtggtagcacttactacaatctatctctcagaagtcgggtcaccatatcagtagacacctctaagaaccagttctccctgaggctgagctctgtgaccgctgcggacacggccatgtattactgtgcgagaattactacgactacctatgctatggactactggggccaagggaccacggtcaccgtctcc

SEQ ID NO: 24 - DNA, 334 bp, Artificial Sequence
  Synthetic construct
gacatcgtgctgacccagtctccagactccctgtctgtgtctctgggcgagagggtcaccgtcaactgcagagccagtgaaagtgttgatggttatggcaatagttttctgcactggttccagcaaaacccaggacagcctcctaaactcctcatttatcttgcatccaacctaaactctggggtccctgcccgattcatgggcagcgggtctgggacagaattcagtctcaccatcagcagcctgcaggctgaagatgtggcagtttattactgtcagcaaaataatgtggacccgtggacctttggccaggggaccaagctggagatcaacc

SEQ ID NO: 25 - DNA, 333 bp, Artificial Sequence
  Synthetic construct
gacatcgtgctgacccagtctccagactccctgtctgtgtctctgggcgagagggtcaccgtcaactgcagagccagtgaaagtgttgatggttatggcaatagttttctgcactggttccagcaaaacccaggacagcctcctaaactcctcatttatcttgcatccaacctaaactctggggtccctgcccgattcatgggcagcgggtctcggacagaattcagtctcaccatcagcagcctgcaggctgaagatgtggcagtttattactgtcagcaaaataatgtggacccgtggacctttggccaggggaccaagctggagatcaaa

SEQ ID NO: 26 - DNA, 334 bp, Artificial Sequence
  Synthetic construct
gaaatagtgttgacgcagtctccatcctccctgtctgcatctgtaggagacagagtcaccatcacttgcagagccagtgaaagtgttgatggttatggcaatagttttctgcactggtatcagcagaaaccagggaaagcccctaagctcctgatctatcttgcatccaacctaaactctggggtcccatcaaggttcagtggcagtggatctgggacagatttcactctcaccatcagcagtctgcaacctgaagattttgcaacttactactgtcagcaaaataatgtggacccgtggacttttggccaggggaccaagctggagatcaaac

SEQ ID NO: 27 - DNA, 333 bp, Artificial Sequence
  Synthetic construct
gaaatagtgttgacgcagtctccatcctccctgtctgcatctgtaggagacagagtcaccatcacttgcagagccagtgaaagtgttgatggttatggcaatagttttctgcactggtttcagcagaaaccagggaaagcccctaagctcctgatctatcttgcatccaacctaaactctggggtcccatcaaggttcagtggcagtggatctcggacagatttcactctcaccatcagcagtctgcaacctgaagattttgcaacttactactgtcagcaaaataatgtggacccgtggacttttggccaggggaccaagctggagatcaaa

SEQ ID NO: 28 - DNA, 350 bp, Artificial Sequence
  Synthetic construct
gaaattgtgctgactcagtctccactctccctgcccgtcacccttggacagccggcctccatctcctgcagagccagtgaaagtgttgatggttatggcaatagttttctgcactggtttcagcagaggccaggccaatctccaaggctcctaatttatcttgcatccaacctaaactctggggtcccagacagattcagcggcagcggatcaaggactgatttcacactgaaaatcagcagagtggaggctgaggatgttggggtttattactgccagcaaaataatgtggacccgtggacgttcggcggagggaccaaagtggagatcaaacgtgagtggatcccgcg

SEQ ID NO: 29 - DNA, 333 bp, Artificial Sequence
  Synthetic construct
gacatcgtgatgacccagtctccagactccctggctgtgtctctgggcgagagggccaccatcaactgcagagccagtgaaagtgttgatggttatggcaatagttttctgcactggtatcagcagaaaccgggacagcctcctaagttgctcatttaccttgcatccaacctaaactctggggtccctgaccgattcagtggcagcgggtctgggacagatttcactctcaccatcagcagcctgcaggccgaagatgtggcagtgtattactgtcagcaaaataatgtggacccgtggacttttggccaggggaccaagctggagatcaaa

SEQ ID NO: 30 - DNA, 351 bp, Artificial Sequence
  Synthetic construct
caagtgcagctgcaggagtcgggaccaggactggtgaagccttcggagaccctgtccctcacctgcactgtctctggtgactccatcactagtggttactggaactggatccggcagcccccagggagggcactggagtacatgggatacataagttacagtggtagcacttactacaatctatctctcagaagtcggatcaccatatcaagagacacctctaagaaccagtactccctgaggctgagctctgtgaccgctgcggacacggccatgtattactgtgcgctgattactacgactacctatgctatggactactggggccaagggaccacggtcaccgtctcc

SEQ ID NO: 31 - DNA, 351 bp, Artificial Sequence
  Synthetic construct
caagtgcagctgcaggagtcgggaccaggactggtgaagccttcggagaccctgtccctcacctgcactgtctctggtgactccatcactagtggttactggaactggatccggaagcccccagggagggcactggagtacatgggatacataagttacagtggtagcacttactacaatctatctctcagaagtcggatcaccatatcaagagacacctctaagaaccagtactccctgaggctgagctctgtgaccgctgcggacacggccatgtattactgtgcgctgattactacgactacctatgctatggactactggggccaagggaccacggtcaccgtctcc

SEQ ID NO: 32 - DNA, 354 bp, Artificial Sequence
  Synthetic construct
caagtgcagctgcaggagtcgggaccaggactggtgaagccttcggagaccctgtccctcacctgcactgtctctggtgactccatcagtagtggttactggaactggatccggcagcccccagggagggcactggagtacatgggatacataagttacagtggtagcacttactacaatctatctctcagaagtcggatcaccatatcaagagacacctctaagaaccagtactccctgaggctgagctctgtgaccgctgcggacacggccatgtattactgtgcgctgattactacgactacctatgctatggactactggggccaagggaccacggtcaccgtctcctca

SEQ ID NO: 33 - DNA, 354 bp, Artificial Sequence
  Synthetic construct
caagtgcagctgcaggagtcgggaccaggactggtgaagccttcggagaccctgtccctcacctgcactgtctctggtgactccatcaccagtggttactggaactggatccggcagcccccagggagggcactggagtggatgggatacataagttacagtggtagcacttactacaatctatctctcagaagtcggatcaccatatcaagagacacctctaagaaccagtactccctgaggctgagctctgtgaccgctgcggacacggccatgtattactgtgcgctgattactacgactacctatgctatggactactggggccaagggaccacggtcaccgtctcctca

SEQ ID NO: 34 - DNA, 351 bp, Artificial Sequence
  Synthetic construct
caagtgcagctgcaggagtcgggaccaggactggtgaagccttcggagaccctgtccctcacctgcactgtctctggtgactccatcaccagtggttactggaactggatccggcagcccccagggagggcactggagtacataggatacataagttacagtggtagcacttactacaatctatctctcagaagtcggatcaccatatcaagagacacctctaagaaccagtactccctgaggctgagctctgtgaccgctgcggacacggccatgtattactgtgcgctgattactacgactacctatgctatggactactggggccaagggaccacggtcaccgtctcc

SEQ ID NO: 35 - DNA, 351 bp, Artificial Sequence
  Synthetic construct
caagtgcagctgcaggagtcgggaccaggactggtgaagccttcggagaccctgtccctcacctgcactgtctctggtgactccatcaccagtggttactggaactggatccggcagcccccagggagggcactggagtacatgggatacataagttacagtggtagcacttactacaatctatctctcagaagtcgggtcaccatatcaagagacacctctaagaaccagtactccctgaggctgagctctgtgaccgctgcggacacggccatgtattactgtgcgctgattactacgactacctatgctatggactactggggccaagggaccacggtcaccgtctcc

SEQ ID NO: 36 - DNA, 351 bp, Artificial Sequence
  Synthetic construct
caagtgcagctgcaggagtcgggaccaggactggtgaagccttcggagaccctgtccctcacctgcactgtctctggtgactccatcaccagtggttactggaactggatccggcagcccccagggagggcactggagtacatgggatacataagttacagtggtagcacttactacaatctatctctcagaagtcggatcaccatatcagtggacacctctaagaaccagtactccctgaggctgagctctgtgaccgctgcggacacggccatgtattactgtgcgctgattactacgactacctatgctatggactactggggccaagggaccacggtcaccgtctcc

SEQ ID NO: 37 - DNA, 351 bp, Artificial Sequence
  Synthetic construct
caagtgcagctgcaggagtcgggaccaggactggtgaagccttcggagaccctgtccctcacctgcactgtctctggtgactccatcaccagtggttactggaactggatccggcagcccccagggagggcactggagtacatgggatacataagttacagtggtagcacttactacaatctatctctcagaagtcggatcaccatatcaagagacacctctaagaaccagttctccctgaggctgagctctgtgaccgctgcggacacggccatgtattactgtgcgctgattactacgactacctatgctatggactactggggccaagggaccacggtcaccgtctcc

SEQ ID NO: 38 - DNA, 351 bp, Artificial Sequence
  Synthetic construct
caagtgcagctgcaggagtcgggaccaggactggtgaagccttcggagaccctgtccctcacctgcactgtctctggtgactccatcaccagtggttactggaactggatccggcagcccccagggagggcactggagtacatgggatacataagttacagtggtagcacttactacaatctatctctcagaagtcggatcaccatatcaagagacacctctaagaaccagtactccctgaggctgagctctgtgaccgctgcggacacggccatgtattactgtgcgagaattactacgactacctatgctatggactactggggccaagggaccacggtcaccgtctcc

SEQ ID NO: 39 - DNA, 333 bp, Artificial Sequence
  Synthetic construct
gaaatagtgttgacgcagtctccatcctccctgtctgcatctgtaggagacagagtcaccatcacttgcagagccagtgaaagtgttgatggttatggcaatagttttctgcactggtttcagcagaaaccagggaaagcccctaagctcctgatctatcttgcatccaacctaaactctggggtcccatcaaggttcagtggcagtggatctgggacagatttcactctcaccatcagcagtctgcaacctgaagattttgcaacttactactgtcagcaaaataatgtggacccgtggacttttggccaggggaccaagctggagatcaaa

SEQ ID NO: 40 - DNA, 334 bp, Artificial Sequence
  Synthetic construct
gaaatagtgttgacgcagtctccatcctccctgtctgcatctgtaggagacagagtcaccatcacttgcagagccagtgaaagtgttgatggttatggcaatagttttctgcactggtatcagcagaaaccagggaaagcccctaagctcctgatctatcttgcatccaacctaaactctggggtcccatcaaggttcagtggcagtggatctcggacagatttcactctcaccatcagcagtctgcaacctgaagattttgcaacttactactgtcagcaaaataatgtggacccgtggacttttggccaggggaccaagctggagatcaaac

SEQ ID NO: 41 - protein, 15 aa, Artificial Sequence
  Synthetic construct
RASESVDGYGNSFLH

SEQ ID NO: 42 - protein, 7 aa, Artificial Sequence
  Synthetic construct
LASNLNS

SEQ ID NO: 43 - protein, 9 aa, Artificial Sequence
  Synthetic construct
QQNNVDPWT

SEQ ID NO: 44 - protein, 10 aa, Artificial Sequence
  Synthetic construct
GDSITSGYWN

SEQ ID NO: 45 - protein, 9 aa, Artificial Sequence
  Synthetic construct
YISYSGSTY

SEQ ID NO: 46 - protein, 10 aa, Artificial Sequence
  Synthetic construct
ITTTTYAMDY

SEQ ID NO: 47 - protein, 5 aa, Artificial Sequence
  Synthetic construct
SGYWN

SEQ ID NO: 48 - protein, 16 aa, Artificial Sequence
  Synthetic construct
YISYSGSTYYNLSLRS

SEQ ID NO: 49 - protein, 118 aa, Artificial Sequence
  Synthetic construct
QVQLQESGPGLVKPSETLSLTCTVSGDSISSGYWNWIRQPPGRALEWIGYISYSGSTYYNLSLRSRVTISVDTSKNQFSLRLSSVTAADTAMYYCARITTTTYAMDYWGQGTTVTVSS

SEQ ID NO: 50 - protein, 30 aa, Artificial Sequence
  Synthetic construct
QVQLQESGPGLVKPSETLSLTCTVSGDSIS

SEQ ID NO: 51 - protein, 14 aa, Artificial Sequence
  Synthetic construct
WIRQPPGRALEWIG

SEQ ID NO: 52 - protein, 32 aa, Artificial Sequence
  Synthetic construct
RVTISVDTSKNQFSLRLSSVTAADTAMYYCAR

SEQ ID NO: 53 - protein, 11 aa, Artificial Sequence
  Synthetic construct
WGQGTTVTVSS

SEQ ID NO: 54 - protein, 122 aa, Homo sapiens
QVQLQESGPGLVKPSETLSLTCTVSGDSISSYYWSWIRQPPGRALEWIGYIYHGGSTNYSPSLKSRVTISVDTSKNQFSLRLSSVTAADTAMYYCARDRHCSGGTCYGMDVWGQGTTVTVSS

SEQ ID NO: 55 - DNA, 57 bp, Artificial Sequence
  Synthetic construct
atgaaacatctgtggttcttccttctgctggtggcagctcccagatgggtcctgtcc

SEQ ID NO: 56 - protein, 19 aa, Artificial Sequence
  Synthetic construct
MKHLWFFLLLVAAPRWVLS

SEQ ID NO: 57 - DNA, 90 bp, Artificial Sequence
  Synthetic construct
caggtgcagctgcaggagtcgggcccaggactggtgaagccttcggagaccctgtccctcacctgcactgtctctggtgactccatcagt

SEQ ID NO: 58 - DNA, 15 bp, Artificial Sequence
  Synthetic construct
agtggttactggaac

SEQ ID NO: 59 - DNA, 39 bp, Artificial Sequence
  Synthetic construct
atccggcagcccccagggagggcactggagtggatagga

SEQ ID NO: 60 - DNA, 48 bp, Artificial Sequence
  Synthetic construct
tacataagttacagtggtagcacttactacaatctatctctcagaagt

SEQ ID NO: 61 - DNA, 96 bp, Artificial Sequence
  Synthetic construct
cgggtcaccatatcagtagacacgtctaagaaccagttctccctgaggctgagctctgtgaccgctgcggacacggccatgtattactgtgcgaga

SEQ ID NO: 62 - DNA, 30 bp, Artificial Sequence
  Synthetic construct
attactacgactacctatgctatggactac

SEQ ID NO: 63 - DNA, 30 bp, Artificial Sequence
  Synthetic construct
tggggccaagggaccacggtcaccgtctcc

SEQ ID NO: 64 - DNA, 453 bp, Artificial Sequence
  Synthetic construct
cacgccaagcttgccgccaccatgaaacatctgtggttcttccttctgctggtggcagctcccagatgggtcctgtcccaagtgcagctgcaggagtcgggaccaggactggtgaagccttcggagaccctgtccctcacctgcactgtctctggtgactccatcagtagtggttactggaactggatccggcagcccccagggagggcactggagtggataggatacataagttacagtggtagcacttactacaatctatctctcagaagtcgggtcaccatatcagtagacacctctaagaaccagttctccctgaggctgagctctgtgaccgctgcggacacggccatgtattactgtgcgagaattactacgactacctatgctatggactactggggccaagggaccacggtcaccgtctcctcagcctccaccaagggcccatcg

SEQ ID NO: 65 - protein, 151 aa, Artificial Sequence
  Synthetic construct
HAKLAATMKHLWFFLLLVAAPRWVLSQVQLQESGPGLVKPSETLSLTCTVSGDSISSGYWNWIRQPPGRALEWIGYISYSGSTYYNLSLRSRVTISVDTSKNQFSLRLSSVTAADTAMYYCARITTTTYAMDYWGQGTTVTVSSASTKGPS

SEQ ID NO: 66 - protein, 23 aa, Artificial Sequence
  Synthetic construct
DIVLTQSPDSLSVSLGERVTVNC

SEQ ID NO: 67 - protein, 15 aa, Artificial Sequence
  Synthetic construct
WFQQNPGQPPKLLIY

SEQ ID NO: 68 - protein, 32 aa, Artificial Sequence
  Synthetic construct
GVPARFMGSGSGTEFSLTISSLQAEDVAVYYC

SEQ ID NO: 69 - protein, 10 aa, Artificial Sequence
  Synthetic construct
FGQGTKLEIN

SEQ ID NO: 70 - protein, 113 aa, Artificial Sequence
  Synthetic construct
DIVLTQSPDSLSVSLGERVTVNCKLSQSVLHSSNKQNYLAWFQQNPGQPPKLLIYWASARQSGVPARFMGSGSGTEFSLTISSLQAEDVAVYYCQQYYDSTYTFGQGTKLEIN

SEQ ID NO: 71 - DNA, 66 bp, Artificial Sequence
  Synthetic construct
atggacatgagggtccctgctcagctcctggggctcctgcagctctggctctccggcgccagatgt

SEQ ID NO: 72 - protein, 22 aa, Artificial Sequence
  Synthetic construct
MDMRVPAQLLGLLQLWLSGARC

SEQ ID NO: 73 - DNA, 69 bp, Artificial Sequence
  Synthetic construct
gacatcgtgctgacccagtctccagactccctgtctgtgtctctgggcgagagggtcaccgtcaactgc

SEQ ID NO: 74 - DNA, 45 bp, Artificial Sequence
  Synthetic construct
agagccagtgaaagtgttgatggttatggcaatagttttctgcac

SEQ ID NO: 75 - DNA, 21 bp, Artificial Sequence
  Synthetic construct
cttgcatccaacctaaactct

SEQ ID NO: 76 - DNA, 96 bp, Artificial Sequence
  Synthetic construct
ggggtccctgcccgattcatgggcagcgggtctgggacagaattcagtctcaccatcagcagcctgcaggctgaagatgtggcagtttattactgt

SEQ ID NO: 77 - DNA, 27 bp, Artificial Sequence
  Synthetic construct
cagcaaaataatgtggacccgtggacg

SEQ ID NO: 78 - DNA, 30 bp, Artificial Sequence
  Synthetic construct
tttggccaggggaccaagctggagatcaac

SEQ ID NO: 79 - protein, 111 aa, Artificial Sequence; identical to SEQ ID NO: 7
  Synthetic construct

SEQ ID NO: 80 - protein, 23 aa, Artificial Sequence
  Synthetic construct
EIVLTQSPSSLSASVGDRVTITC

SEQ ID NO: 81 - protein, 15 aa, Artificial Sequence
  Synthetic construct
WFQQKPGKAPKLLIY

SEQ ID NO: 82 - protein, 32 aa, Artificial Sequence
  Synthetic construct
GVPSRFSGSGSRTDFTLTISSLQPEDFATYYC

SEQ ID NO: 83 - protein, 10 aa, Artificial Sequence
  Synthetic construct
FGQGTKLEIK

SEQ ID NO: 84 - protein, 109 aa, Artificial Sequence
  Synthetic construct
EIVLTQSPSSLSASVGDRVTITCRLKSQSISSYLNWFQQKPGKAPKLLIYAASSLQSGVPSRFSGSGSRTDFTLTISSLQPEDFATYYCQQSYSTLMYTFGQGTKLEIK

SEQ ID NO: 85 - DNA, 69 bp, Artificial Sequence
  Synthetic construct
gaaatagtgttgacgcagtctccatcctccctgtctgcatctgtaggagacagagtcaccatcacttgc

SEQ ID NO: 86 - DNA, 45 bp, Artificial Sequence
  Synthetic construct
tggtttcagcagaaaccagggaaagcccctaagctcctgatctat

SEQ ID NO: 87 - DNA, 96 bp, Artificial Sequence
  Synthetic construct
ggggtcccatcaaggttcagtggcagtggatctcggacagatttcactctcaccatcagcagtctgcaacctgaagattttgcaacttactactgt

SEQ ID NO: 88 - DNA, 30 bp, Artificial Sequence
  Synthetic construct
tttggccaggggaccaagctggagatcaaa

SEQ ID NO: 89 - DNA, 399 bp, Artificial Sequence
  Synthetic construct
atggacatgagggtccctgctcagctcctggggctcctgcagctctggctctccggcgccagatgtgacatcgtgctgacccagtctccagactccctgtctgtgtctctgggcgagagggtcaccgtcaactgcagagccagtgaaagtgttgatggttatggcaatagttttctgcacagagccagtgaaagtgttgatggttatggcaatagttttctgcaccttgcatccaacctaaactctggggtccctgcccgattcatgggcagcgggtctgggacagaattcagtctcaccatcagcagcctgcaggctgaagatgtggcagtttattactgtcagcaaaataatgtggacccgtggacgtttggccaggggaccaagctggagatcaac

SEQ ID NO: 90 - DNA, 399 bp, Artificial Sequence
  Synthetic construct
atggacatgagggtccccgctcagctcctggggctcctgctactctggctccgaggtgccagatgtgaaatagtgttgacgcagtctccatcctccctgtctgcatctgtaggagacagagtcaccatcacttgcagagccagtgaaagtgttgatggttatggcaatagttttctgcactggtttcagcagaaaccagggaaagcccctaagctcctgatctatcttgcatccaacctaaactctggggtcccatcaaggttcagtggcagtggatctcggacagatttcactctcaccatcagcagtctgcaacctgaagattttgcaacttactactgtcagcaaaataatgtggacccgtggacgtttggccaggggaccaagctggagatcaaa

SEQ ID NO: 91 - DNA, 393 bp, Artificial Sequence
  Synthetic construct
atgaggctccctgctcagctcctggggctgctaatgctctgggtcccaggatccagtggggaaattgtgctgactcagtctccactctccctgcccgtcacccttggacagccggcctccatctcctgcagagccagtgaaagtgttgatggttatggcaatagttttctgcactggtttcagcagaggccaggccaatctccaaggcgcctaatttatcttgcatccaacctaaactctggggtcccagacagattcagcggcagtgggtcaggcactgatttcacactgaaaatcagcagggtggaggctgaggatgttggggtttattactgccagcaaaataatgtggacccgtggacgttcggcggagggaccaaggtggagatcaaa

SEQ ID NO: 92 - DNA, 399 bp, Artificial Sequence
  Synthetic construct
atggacatgagggtccctgctcagctcctggggctcctgcagctctggctctcaggggccagatgtgacatcgtgatgacccagtctccagactccctggctgtgtctctgggcgagagggccaccatcaactgcagagccagtgaaagtgttgatggttatggcaatagttttctgcactggtatcagcagaaaccgggacagcctcctaagttgctcatttaccttgcatccaacctaaactctggggtccctgaccgattcagtggcagcgggtctgggacagatttcactctcaccatcagcagcctgcaggccgaagatgtggcagtgtattactgtcagcaaaataatgtggacccgtggacgtttggccaggggaccaagctggagatcaaa

SEQ ID NO: 93 - DNA, 394 bp, Artificial Sequence
  Synthetic construct
atggtgttgcagacccaggtcttcatttctctgttgctctggatctctggggcttacggggacatcgtgctgacccagtctccagactccctgtctgtgtctctgggcgagagggtcaccgtcaactgcagagccagtgaaagtgttgatggttatggcaatagttttctgcactggttccagcaaaacccaggacagcctcctaaactcctcatttatcttgcatccaacctaaactctggggtccctgcccgattcatgggcagcgggtctgggacagaattcagtctcaccatcagcagcctgcaggctgaagatgtggcagtttattactgtcagcaaaataatgtggacccgtggacctttggccaggggaccaagctggagatcaacc

SEQ ID NO: 94 - protein, 131 aa, Artificial Sequence
  Synthetic construct
MVLQTQVFISLLLWISGAYGDIVLTQSPDSLSVSLGERVTVNCRASESVDGYGNSFLHWFQQNPGQPPKLLIYLASNLNSGVPARFMGSGSGTEFSLTISSLQAEDVAVYYCQQNNVDPWTFGQGTKLEIN

SEQ ID NO: 95 - DNA, 400 bp, Artificial Sequence
  Synthetic construct
atggacatgagggtccccgctcagctcctggggctcctgctactctggctccgaggtgccagatgtgaaatagtgttgacgcagtctccatcctccctgtctgcatctgtaggagacagagtcaccatcacttgcagagccagtgaaagtgttgatggttatggcaatagttttctgcactggtatcagcagaaaccagggaaagcccctaagctcctgatctatcttgcatccaacctaaactctggggtcccatcaaggttcagtggcagtggatctgggacagatttcactctcaccatcagcagtctgcaacctgaagattttgcaacttactactgtcagcaaaataatgtggacccgtggacttttggccaggggaccaagctggagatcaaac

SEQ ID NO: 96 - DNA, 400 bp, Artificial Sequence
  Synthetic construct
gtttgatctccagcttggtcccctggccaaaagtccacgggtccacattattttgctgacagtagtaagttgcaaaatcttcaggttgcagactgctgatggtgagagtgaaatctgtcccagatccactgccactgaaccttgatgggaccccagagtttaggttggatgcaagatagatcaggagcttaggggctttccctggtttctgctgataccagtgcagaaaactattgccataaccatcaacactttcactggctctgcaagtgatggtgactctgtctcctacagatgcagacagggaggatggagactgcgtcaacactatttcacatctggcacctcggagccagagtagcaggagccccaggagctgagcggggaccctcatgtccat

SEQ ID NO: 97 - protein, 133 aa, Artificial Sequence
  Synthetic construct
MDMRVPAQLLGLLLLWLRGARCEIVLTQSPSSLSASVGDRVTITCRASESVDGYGNSFLHWYQQKPGKAPKLLIYLASNLNSGVPSRFSGSGSGTDFTLTISSLQPEDFATYYCQQNNVDPWTFGQGTKLEIK

SEQ ID NO: 98 - protein, 17 aa, Artificial Sequence
  Synthetic construct
IIWFQPLLIYGGRTFYF

SEQ ID NO: 99 - protein, 17 aa, Homo sapiens
ILWFQPRLIYGGGTFYF

SEQ ID NO: 100 - protein, 17 aa, Homo sapiens
IMWFQPLLIYGGGTFYF

SEQ ID NO: 101 - protein, 17 aa, Homo sapiens
ILWFQPLLIYGGGTFFF

SEQ ID NO: 102 - protein, 17 aa, Homo sapiens
ILWYQPLLIYGGGTFYF

SEQ ID NO: 103 - protein, 17 aa, Artificial Sequence
  Synthetic construct
ILWYQPLLIYGGETFYF

SEQ ID NO: 104 - protein, 112 aa, Homo sapiens
AEIVLTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWLTFGGGTKVEIK

SEQ ID NO: 105 - protein, 113 aa, Homo sapiens
GDIVLTQSPLSLPVTLGQPASISCRSSQNLVYSDGNTYLNWFQQRPGQSPRRLIYKVSNRDSGVPDSFSGSGSGTDFTLTISRVEAEDVGIYYCMQGTRWPYTFGEGTKLEIK

SEQ ID NO: 106 - protein, 112 aa, Homo sapiens
DIVLTQSPLSLPVTLGQPASISCRSSQNLVYSDGNTYLNWFQQRPGQSPRRLIYKVSNRDSGVPDSFSGSGSGTDFTLTISRVEAEDVGIYYCMQGTRWPYTFGEGTKLEIK

SEQ ID NO: 107 - protein, 113 aa, Homo sapiens
DIVMTQTPLSLSVSPGQAASISCKSSQSLQHPNGKTYLYWFLQKPGQPPQLLIYELSRRFSGVPARFSGSGSGTEFTLKISRVEAEDVGVYYCMETMELRSITFGQGTRLEIK

SEQ ID NO: 108 - protein, 113 aa, Homo sapiens
AEIVLTQSPLSLPVTLGQPASISCRSSQSLVDSNGRIYLAWFQQRPGQSPRRLIYPVSKRDSGVPDRFSGSGSGTEFTLKISRVEAEDVGVYYCMQHTHWPHTFGQGTKLEIK

SEQ ID NO: 109 - protein, 112 aa, Homo sapiens
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIK

SEQ ID NO: 110 - protein, 113 aa, Homo sapiens
AEIVLTQSPLSLPVTLGQPASISCRSSQSLVYSDGNTYLNWFQQRPGQSPRRLIYKVSNWDSGVPDRFSGSGSGTDFALKISRVEAEDVGVYYCMQGTHWPLTFGQGTRLEIK

SEQ ID NO: 111 - protein, 113 aa, Homo sapiens
AEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGQGTKLEIK

SEQ ID NO: 112 - protein, 113 aa, Homo sapiens
AEIVLTQSPLSLPVTPGEPASISCKSSQSLLHMNGANYLDWYLQKPGQSPQLLIYLGSNRASGVPDRFSGSGSETDFTLKITRVEADDVGVYYCMQSLQNPLTFGGGTKVEIR

SEQ ID NO: 113 - protein, 113 aa, Homo sapiens
AEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIK

SEQ ID NO: 114 - protein, 112 aa, Homo sapiens
DIVMTQTPLSSPVTLGQPASISCRSSQSLVHSDGNTYLNWFHQRPGQPPRLLIYKISSRFSGVPDRFSGSGAGTDFTLKISRVEAEDVGVYYCMQATQFPHTFGQGTKLEIK

SEQ ID NO: 115 - protein, 112 aa, Homo sapiens
EIVLTQSPLSLPVTPGEPASISCRSSQSLLHSNGYTYLDWYLQKPGQSPQLLIYLGSNRASGVPDKFSGSGSGTDFTLKISRVEAEDVGVYYCMQALETPQTFGQGTRLEIK

SEQ ID NO: 116 - protein, 112 aa, Homo sapiens
DIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKVEIK

SEQ ID NO: 117 - protein, 106 aa, Homo sapiens
DIVMTQTPLSLSVTPGQPASISCKSSQSLLHSDGKTYLYWFLQKPGQPPQLLIYEVSNRFSGVPDRFSGSGSGTDFTLKISRVEAEDVGVYYCMQSIQLPRWTFGQ

SEQ ID NO: 118 - protein, 112 aa, Homo sapiens
EIVLTQSPLSLSVTPGEPASISCRSSQSLLHSNGYNYLDWYLQKPGQSPQLLIYVGSSRASGVPDRFSGSGSGTDFTLKISRVEAEDVGVYYCMQTTHWPLYTFGQGTKVEI

SEQ ID NO: 119 - protein, 106 aa, Homo sapiens
EIVLTQTPLSLSVTPGQPASISCKSSQSLLHSDGKTYFYWYLQKPGQSPQLLIYEVSNRFSGVPDRFSGSGSGTDFTLKISRVEAEDVGVYYCMQSIQLPRWTFGQ

SEQ ID NO: 120 - protein, 106 aa, Homo sapiens
DIVMTQTPLSLSVTPGQPASISCKSSQSLLHSDGKTYLYWFLQKPGQPPQLLIYEVSNRFSGVPDRFSGSGSGTDFTLKISRVEAEDVGVYYCMQSIQPPRWTFGQ

SEQ ID NO: 121 - protein, 113 aa, Homo sapiens
EIVLTQSPLSLPVTPGEPAPISCRSSQSLLHSDGYNYLDWYVQKPGQSPQLLIYLASNRASGVPDRFSGSGSGTEFTLKISRVEAEDVGVYYCTQALQTPGWTFGQGTKVEIK

SEQ ID NO: 122 - protein, 112 aa, Homo sapiens
EIVLTQSHLSLPVTPGEPASISCRSSQSFLHSNGYNYLDWYLQKPGQSPQLLIYLGSNRASRVPDRFSGSGSGTDFTLKISRVEAEDVGVYYCMQALQTQGTFGQGTKVEIK

SEQ ID NO: 123 - protein, 112 aa, Homo sapiens
DIVMTQSPLSLPVTPGEPASISCRSSQSLLHSDGYNSLDWFLQRPGQSPQLLIYLGSNRASGVPDRFSGSGSGTDFTLKISRVEAEDVGVYYCMQVLQTPFTFGPGTKVDIK

SEQ ID NO: 124 - protein, 111 aa, Homo sapiens
EIVLTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWLTFGGGTKVEIK

SEQ ID NO: 125 - protein, 112 aa, Homo sapiens
  Synthetic construct
DIVLTQSPLSLPVTLGQPASISCRSSQNLVYSDGNTYLNWFQQRPGQSPRRLIYKVSNRDSGVPDSFSGSGSGIDFTLTISRVEAEDVGIYYCMQGTRWPYTFGEGTKLEIK

SEQ ID NO: 126 - protein, 113 aa, Homo sapiens; identical to SEQ ID NO: 107

SEQ ID NO: 127 - protein, 112 aa, Homo sapiens
EIVLTQSPLSLPVTLGQPASISCRSSQSLVDSNGRIYLAWFQQRPGQSPRRLIYPVSKRDSGVPDRFSGSGSGTEFTLKISRVEAEDVGVYYCMQHTHWPHTFGQGTKLEIK

SEQ ID NO: 128 - protein, 112 aa, Homo sapiens; identical to SEQ ID NO: 109

SEQ ID NO: 129 - protein, 112 aa, Homo sapiens
EIVLTQSPLSLPVTLGQPASISCRSSQSLVYSDGNTYLNWFQQRPGQSPRRLIYKVSNWDSGVPDRFSGSGSGTDFALKISRVEAEDVGVYYCMQGTHWPLTFGQGTRLEIK

SEQ ID NO: 130 - protein, 112 aa, Homo sapiens
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGQGTKLEIK

SEQ ID NO: 131 - protein, 112 aa, Homo sapiens
EIVLTQSPLSLPVTPGEPASISCKSSQSLLHMNGANYLDWYLQKPGQSPQLLIYLGSNRASGVPDRFSGSGSETDFTLKITRVEADDVGVYYCMQSLQNPLTFGGGTKVEIR

SEQ ID NO: 132 - protein, 112 aa, Homo sapiens
EIVLTQSPLSLPVTPGEPASISCRSSQSLLHSNGYNYLDWYLQKPGQSPQLLIYLGSNRASGVPDRFSGSGSGIDFTLKISRVEAEDVGVYYCMQALQTPRTFGQGTKVEIK

SEQ ID NO: 133 - protein, 112 aa, Homo sapiens; identical to SEQ ID NO: 114

SEQ ID NO: 134 - protein, 112 aa, Homo sapiens; identical to SEQ ID NO: 115

SEQ ID NO: 135 - protein, 107 aa, Homo sapiens
DIVMTQTPLSLSVTPGQPASISCKSSQSLLHSDGKTYLYWFLQKPGQPPQLLIYEVSNRFSGVPDRFSGSGSGTDFTLKISRVEAEDVGVYYCMQSIQLPRWTFGQG

SEQ ID NO: 136 - protein, 112 aa, Homo sapiens; identical to SEQ ID NO: 123

SEQ ID NO: 137 - protein, 107 aa, Homo sapiens
EIVLTQTPLSLSVTPGQPASISCKSSQSLLHSDGKTYFYWYLQKPGQSPQLLIYEVSNRFSGVPDRFSGSGSGTDFTLKISRVEAEDVGVYYCMQSIQLPRWTFGQG

SEQ ID NO: 138 - protein, 107 aa, Homo sapiens
DIVMTQTPLSLSVTPGQPASISCKSSQSLLHSDGKTYLYWFLQKPGQPPQLLIYEVSNRFSGVPDRFSGSGSGTDFTLKISRVEAEDVGVYYCMQSIQPPRWTFGQG

SEQ ID NO: 139 - protein, 113 aa, Homo sapiens; identical to SEQ ID NO: 121

SEQ ID NO: 140 - protein, 112 aa, Homo sapiens; identical to SEQ ID NO: 122

SEQ ID NO: 141 - protein, 113 aa, Homo sapiens
EIVLTQSPLSLSVTPGEPASISCRSSQSLLHSNGYNYLDWYLQKPGQSPQLLIYVGSSRASGVPDRFSGSGSGTDFTLKISRVEAEDVGVYYCMQTTHWPLYTFGQGTKVEIK

SEQ ID NO: 142 - protein, 122 aa, Homo sapiens; identical to SEQ ID NO: 54

SEQ ID NO: 143 - protein, 111 aa, Artificial Sequence; identical to SEQ ID NO: 6
  Synthetic construct

SEQ ID NO: 144 - protein, 111 aa, Artificial Sequence; identical to SEQ ID NO: 124
  Synthetic construct

SEQ ID NO: 145 - protein, 113 aa, Artificial Sequence
  Synthetic construct
DIVMTQSPDSLAVSLGERATINCKSSQSVLYSSNNNNYLAWYQQKPGQPPKLLIYWASTRESGVPDRFSGSGSGTDFTLTISSLQAEDVAVYYCQQYYSTSYTFGQGTKLEIK

SEQ ID NO: 146 - protein, 111 aa, Artificial Sequence
  Synthetic construct
EIVLTQSPLSLPVTLGQPASISCRASESVDGYGNSFLHWFQQRPGQSPRRLIYLASNLNSGVPDRFSGSGSGTDFTLKISRVEAEDVGVYYCQQNNVDPWTFGGGTKVEIK

SEQ ID NO: 147 - protein, 23 aa, Artificial Sequence
  Synthetic construct
EIVLTQSPLSLPVTLGQPASISC

SEQ ID NO: 148 - protein, 15 aa, Artificial Sequence
  Synthetic construct
WFQQRPGQSPRRLIY

SEQ ID NO: 149 - protein, 32 aa, Artificial Sequence
  Synthetic construct
GVPDRFSGSGSGTDFTLKISRVEAEDVGVYYC

SEQ ID NO: 150 - protein, 10 aa, Artificial Sequence
  Synthetic construct
FGGGTKVEIK

SEQ ID NO: 151 - DNA, 60 bp, Artificial Sequence
  Synthetic construct
atgaggctccctgctcagctcctggggctgctaatgctctgggtcccaggatccagtggg

SEQ ID NO: 152 - protein, 20 aa, Artificial Sequence
  Synthetic construct
MRLPAQLLGLLMLWVPGSSG

SEQ ID NO: 153 - DNA, 69 bp, Artificial Sequence
  Synthetic construct
gaaattgtgctgactcagtctccactctccctgcccgtcacccttggacagccggcctccatctcctgc

SEQ ID NO: 154 - DNA, 45 bp, Artificial Sequence
  Synthetic construct
tggtttcagcagaggccaggccaatctccaaggcgcctaatttat

SEQ ID NO: 155 - DNA, 96 bp, Artificial Sequence
  Synthetic construct
ggggtcccagacagattcagcggcagtgggtcaggcactgatttcacactgaaaatcagcagggtggaggctgaggatgttggggtttattactgc

SEQ ID NO: 156 - DNA, 30 bp, Artificial Sequence
  Synthetic construct
ttcggcggagggaccaaggtggagatcaaa

SEQ ID NO: 157 - DNA, 66 bp, Artificial Sequence
  Synthetic construct
atggacatgagggtccctgctcagctcctggggctcctgcagctctggctctcaggtgccagatgt

SEQ ID NO: 158 - DNA, 69 bp, Artificial Sequence
  Synthetic construct
gacatcgtgatgacccagtctccagactccctggctgtgtctctgggtgagagggccaccatcaactgc

SEQ ID NO: 159 - protein, 23 aa, Artificial Sequence
  Synthetic construct
DIVMTQSPDSLAVSLGERATINC

SEQ ID NO: 160 - DNA, 45 bp, Artificial Sequence
  Synthetic construct
tggtaccagcagaaaccgggacagcctcctaagttgctcatttac

SEQ ID NO: 161 - protein, 15 aa, Artificial Sequence
  Synthetic construct
WYQQKPGQPPKLLIY

SEQ ID NO: 162 - DNA, 96 bp, Artificial Sequence
  Synthetic construct
ggggtccctgaccgattcagtggcagcgggtctgggacagatttcactctcaccatcagcagcctgcaggccgaagatgtggcagtgtattactgt

SEQ ID NO: 163 - protein, 32 aa, Artificial Sequence
  Synthetic construct
GVPDRFSGSGSGTDFTLTISSLQAEDVAVYYC

SEQ ID NO: 164 - DNA, 425 bp, Artificial Sequence
  Synthetic construct
aagcttgccgccaccatgaggctccctgctcagctcctggggctgctaatgctctgggtcccagggtccagcggggaaattgtgctgactcagtctccactctccctgcccgtcacccttggacagccggcctccatctcctgcagagccagtgaaagtgttgatggttatggcaatagttttctgcactggtttcagcagaggccaggccaatctccaaggcgcctaatttatcttgcatccaacctaaactctggggtcccagacagattcagcggcagcggatcaggcactgatttcacactgaaaatcagcagagtggaggctgaggatgttggggtttattactgccagcaaaataatgtggacccgtggacgttcggcggagggaccaaagtggagatcaaacgtgagtggatcccgcg

SEQ ID NO: 165 - protein, 142 aa, Artificial Sequence
  Synthetic construct
KLAATMRLPAQLLGLLMLWVPGSSGEIVLTQSPLSLPVTLGQPASISCRASESVDGYGNSFLHWFQQRPGQSPRRLIYLASNLNSGVPDRFSGSGSGTDFTLKISRVEAEDVGVYYCQQNNVDPWTFGGGTKVEIKREWIPR

SEQ ID NO: 166 - DNA, 399 bp, Artificial Sequence
  Synthetic construct
atggacatgagggtccctgctcagctcctggggctcctgcagctctggctctcaggggccagatgtgacatcgtgatgacccagtctccagactccctggctgtgtctctgggcgagagggccaccatcaactgcagagccagtgaaagtgttgatggttatggcaatagttttctgcactggtatcagcagaaaccgggacagcctcctaagttgctcatttaccttgcatccaacctaaactctggggtccctgaccgattcagtggcagcgggtctgggacagatttcactctcaccatcagcagcctgcaggccgaagatgtggcagtgtattactgtcagcaaaataatgtggacccgtggacttttggccaggggaccaagctggagatcaaa

SEQ ID NO: 167 - DNA, 399 bp, Artificial Sequence
  Synthetic construct
tttgatctccagcttggtcccctggccaaaagtccacgggtccacattattttgctgacagtaatacactgccacatcttcggcctgcaggctgctgatggtgagagtgaaatctgtcccagacccgctgccactgaatcggtcagggaccccagagtttaggttggatgcaaggtaaatgagcaacttaggaggctgtcccggtttctgctgataccagtgcagaaaactattgccataaccatcaacactttcactggctctgcagttgatggtggccctctcgcccagagacacagccagggagtctggagactgggtcatcacgatgtcacatctggcccctgagagccagagctgcaggagccccaggagctgagcagggaccctcatgtccat

SEQ ID NO: 168 - protein, 133 aa, Artificial Sequence
  Synthetic construct
MDMRVPAQLLGLLQLWLSGARCDIVMTQSPDSLAVSLGERATINCRASESVDGYGNSFLHWYQQKPGQPPKLLIYLASNLNSGVPDRFSGSGSGTDFTLTISSLQAEDVAVYYCQQNNVDPWTFGQGTKLEIK

SEQ ID NO: 169 - DNA, 351 bp, Artificial Sequence
  Synthetic construct
ggagacggtgactgaggttccttgaccccagtagtccatagcataggtagtcgtagtaatgagcgcacagtaatatgtggctgtgtcctcagtagtcacagaattcaactgcaggtagtactgattcttggatgtgtctcgagtgatggagatgcgacttctgagagatagattgtagtaagtgctaccactgtaacttatgtatcccatgtactcaagtttattccctgggaatttccggatccagttccagtaaccactggtgatggagtcgccagtgacagaacaggtgagggacagagtctgagaaggtttcacgaggctaggtcctgactcctgaagctgcacctc

SEQ ID NO: 170 - DNA, 333 bp, Artificial Sequence
  Synthetic construct
tttgatttccagcttggtgcctccaccgaacgtccacgggtccacattattttgctgacagtaataggttgcagcatcatcagcctccacaggatcaatggtgagggtgaagtctgtcctagacccactgccactgaacctggcagggaccccagagtttaggttggatgcaagatagatgaggagtttgggtggctgtcctggtttctgctggaaccagtgcagaaaactattgccataaccatcaacactttcactggctctgcaggaaatggtggccctctgccccagagacacagccaaagaaactggagattgggtcagcacaatgtt

SEQ ID NO: 171 - DNA, 351 bp, Artificial Sequence
  Synthetic construct
ggagacggtgaccgtggtcccttggccccagtagtccatagcataggtagtcgtagtaattctcgcacagtaatacatggccgtgtccgcagcggtcacagagctcagcctcagggagaactggttcttagaggtgtctactgatatggtgacccgacttctgagagatagattgtagtaagtgctaccactgtaacttatgtatcctatccactccagtgccctccctgggggctgccggatccagttccagtaaccactactgatggagtcaccagagacagtgcaggtgagggacagggtctccgaaggcttcaccagtcctggtcccgactcctgcagctgcacttg

SEQ ID NO: 172 - DNA, 334 bp, Artificial Sequence
  Synthetic construct
ggttgatctccagcttggtcccctggccaaaggtccacgggtccacattattttgctgacagtaataaactgccacatcttcagcctgcaggctgctgatggtgagactgaattctgtcccagacccgctgcccatgaatcgggcagggaccccagagtttaggttggatgcaagataaatgaggagtttaggaggctgtcctgggttttgctggaaccagtgcagaaaactattgccataaccatcaacactttcactggctctgcagttgacggtgaccctctcgcccagagacacagacagggagtctggagactgggtcagcacgatgtc

SEQ ID NO: 173 - DNA, 333 bp, Artificial Sequence
  Synthetic construct
tttgatctccagcttggtcccctggccaaaggtccacgggtccacattattttgctgacagtaataaactgccacatcttcagcctgcaggctgctgatggtgagactgaattctgtccgagacccgctgcccatgaatcgggcagggaccccagagtttaggttggatgcaagataaatgaggagtttaggaggctgtcctgggttttgctggaaccagtgcagaaaactattgccataaccatcaacactttcactggctctgcagttgacggtgaccctctcgcccagagacacagacagggagtctggagactgggtcagcacgatgtc

SEQ ID NO: 174 - DNA, 334 bp, Artificial Sequence
  Synthetic construct
gtttgatctccagcttggtcccctggccaaaagtccacgggtccacattattttgctgacagtagtaagttgcaaaatcttcaggttgcagactgctgatggtgagagtgaaatctgtcccagatccactgccactgaaccttgatgggaccccagagtttaggttggatgcaagatagatcaggagcttaggggctttccctggtttctgctgataccagtgcagaaaactattgccataaccatcaacactttcactggctctgcaagtgatggtgactctgtctcctacagatgcagacagggaggatggagactgcgtcaacactatttc

SEQ ID NO: 175 - DNA, 333 bp, Artificial Sequence
  Synthetic construct
tttgatctccagcttggtcccctggccaaaagtccacgggtccacattattttgctgacagtagtaagttgcaaaatcttcaggttgcagactgctgatggtgagagtgaaatctgtccgagatccactgccactgaaccttgatgggaccccagagtttaggttggatgcaagatagatcaggagcttaggggctttccctggtttctgctgaaaccagtgcagaaaactattgccataaccatcaacactttcactggctctgcaagtgatggtgactctgtctcctacagatgcagacagggaggatggagactgcgtcaacactatttc

SEQ ID NO: 176 - DNA, 350 bp, Artificial Sequence
  Synthetic construct
cgcgggatccactcacgtttgatctccactttggtccctccgccgaacgtccacgggtccacattattttgctggcagtaataaaccccaacatcctcagcctccactctgctgattttcagtgtgaaatcagtccttgatccgctgccgctgaatctgtctgggaccccagagtttaggttggatgcaagataaattaggagccttggagattggcctggcctctgctgaaaccagtgcagaaaactattgccataaccatcaacactttcactggctctgcaggagatggaggccggctgtccaagggtgacgggcagggagagtggagactgagtcagcacaatttc

SEQ ID NO: 177 - DNA, 333 bp, Artificial Sequence
  Synthetic construct
tttgatctccagcttggtcccctggccaaaagtccacgggtccacattattttgctgacagtaatacactgccacatcttcggcctgcaggctgctgatggtgagagtgaaatctgtcccagacccgctgccactgaatcggtcagggaccccagagtttaggttggatgcaaggtaaatgagcaacttaggaggctgtcccggtttctgctgataccagtgcagaaaactattgccataaccatcaacactttcactggctctgcagttgatggtggccctctcgcccagagacacagccagggagtctggagactgggtcatcacgatgtc

SEQ ID NO: 178 - DNA, 351 bp, Artificial Sequence
  Synthetic construct
ggagacggtgaccgtggtcccttggccccagtagtccatagcataggtagtcgtagtaatcagcgcacagtaatacatggccgtgtccgcagcggtcacagagctcagcctcagggagtactggttcttagaggtgtctcttgatatggtgatccgacttctgagagatagattgtagtaagtgctaccactgtaacttatgtatcccatgtactccagtgccctccctgggggctgccggatccagttccagtaaccactagtgatggagtcaccagagacagtgcaggtgagggacagggtctccgaaggcttcaccagtcctggtcccgactcctgcagctgcacttg

SEQ ID NO: 179 - DNA, 351 bp, Artificial Sequence
  Synthetic construct
ggagacggtgaccgtggtcccttggccccagtagtccatagcataggtagtcgtagtaatcagcgcacagtaatacatggccgtgtccgcagcggtcacagagctcagcctcagggagtactggttcttagaggtgtctcttgatatggtgatccgacttctgagagatagattgtagtaagtgctaccactgtaacttatgtatcccatgtactccagtgccctccctgggggcttccggatccagttccagtaaccactagtgatggagtcaccagagacagtgcaggtgagggacagggtctccgaaggcttcaccagtcctggtcccgactcctgcagctgcacttg

SEQ ID NO: 180 - DNA, 354 bp, Artificial Sequence
  Synthetic construct
tgaggagacggtgaccgtggtcccttggccccagtagtccatagcataggtagtcgtagtaatcagcgcacagtaatacatggccgtgtccgcagcggtcacagagctcagcctcagggagtactggttcttagaggtgtctcttgatatggtgatccgacttctgagagatagattgtagtaagtgctaccactgtaacttatgtatcccatgtactccagtgccctccctgggggctgccggatccagttccagtaaccactactgatggagtcaccagagacagtgcaggtgagggacagggtctccgaaggcttcaccagtcctggtcccgactcctgcagctgcacttg

SEQ ID NO: 181 - DNA, 354 bp, Artificial Sequence
  Synthetic construct
tgaggagacggtgaccgtggtcccttggccccagtagtccatagcataggtagtcgtagtaatcagcgcacagtaatacatggccgtgtccgcagcggtcacagagctcagcctcagggagtactggttcttagaggtgtctcttgatatggtgatccgacttctgagagatagattgtagtaagtgctaccactgtaacttatgtatcccatccactccagtgccctccctgggggctgccggatccagttccagtaaccactggtgatggagtcaccagagacagtgcaggtgagggacagggtctccgaaggcttcaccagtcctggtcccgactcctgcagctgcacttg

SEQ ID NO: 182 - DNA, 351 bp, Artificial Sequence
  Synthetic construct
ggagacggtgaccgtggtcccttggccccagtagtccatagcataggtagtcgtagtaatcagcgcacagtaatacatggccgtgtccgcagcggtcacagagctcagcctcagggagtactggttcttagaggtgtctcttgatatggtgatccgacttctgagagatagattgtagtaagtgctaccactgtaacttatgtatcctatgtactccagtgccctccctgggggctgccggatccagttccagtaaccactggtgatggagtcaccagagacagtgcaggtgagggacagggtctccgaaggcttcaccagtcctggtcccgactcctgcagctgcacttg

SEQ ID NO: 183 - DNA, 351 bp, Artificial Sequence
  Synthetic construct
ggagacggtgaccgtggtcccttggccccagtagtccatagcataggtagtcgtagtaatcagcgcacagtaatacatggccgtgtccgcagcggtcacagagctcagcctcagggagtactggttcttagaggtgtctcttgatatggtgacccgacttctgagagatagattgtagtaagtgctaccactgtaacttatgtatcccatgtactccagtgccctccctgggggctgccggatccagttccagtaaccactggtgatggagtcaccagagacagtgcaggtgagggacagggtctccgaaggcttcaccagtcctggtcccgactcctgcagctgcacttg

SEQ ID NO: 184 - DNA, 351 bp, Artificial Sequence
  Synthetic construct
ggagacggtgaccgtggtcccttggccccagtagtccatagcataggtagtcgtagtaatcagcgcacagtaatacatggccgtgtccgcagcggtcacagagctcagcctcagggagtactggttcttagaggtgtccactgatatggtgatccgacttctgagagatagattgtagtaagtgctaccactgtaacttatgtatcccatgtactccagtgccctccctgggggctgccggatccagttccagtaaccactggtgatggagtcaccagagacagtgcaggtgagggacagggtctccgaaggcttcaccagtcctggtcccgactcctgcagctgcacttg

SEQ ID NO: 185 - DNA, 351 bp, Artificial Sequence
  Synthetic construct
ggagacggtgaccgtggtcccttggccccagtagtccatagcataggtagtcgtagtaatcagcgcacagtaatacatggccgtgtccgcagcggtcacagagctcagcctcagggagaactggttcttagaggtgtctcttgatatggtgatccgacttctgagagatagattgtagtaagtgctaccactgtaacttatgtatcccatgtactccagtgccctccctgggggctgccggatccagttccagtaaccactggtgatggagtcaccagagacagtgcaggtgagggacagggtctccgaaggcttcaccagtcctggtcccgactcctgcagctgcacttg

SEQ ID NO: 186 - DNA, 351 bp, Artificial Sequence
  Synthetic construct
ggagacggtgaccgtggtcccttggccccagtagtccatagcataggtagtcgtagtaattctcgcacagtaatacatggccgtgtccgcagcggtcacagagctcagcctcagggagtactggttcttagaggtgtctcttgatatggtgatccgacttctgagagatagattgtagtaagtgctaccactgtaacttatgtatcccatgtactccagtgccctccctgggggctgccggatccagttccagtaaccactggtgatggagtcaccagagacagtgcaggtgagggacagggtctccgaaggcttcaccagtcctggtcccgactcctgcagctgcacttg

SEQ ID NO: 187 - DNA, 333 bp, Artificial Sequence
  Synthetic construct
tttgatctccagcttggtcccctggccaaaagtccacgggtccacattattttgctgacagtagtaagttgcaaaatcttcaggttgcagactgctgatggtgagagtgaaatctgtcccagatccactgccactgaaccttgatgggaccccagagtttaggttggatgcaagatagatcaggagcttaggggctttccctggtttctgctgaaaccagtgcagaaaactattgccataaccatcaacactttcactggctctgcaagtgatggtgactctgtctcctacagatgcagacagggaggatggagactgcgtcaacactatttc

SEQ ID NO: 188 - DNA, 334 bp, Artificial Sequence
  Synthetic construct
gtttgatctccagcttggtcccctggccaaaagtccacgggtccacattattttgctgacagtagtaagttgcaaaatcttcaggttgcagactgctgatggtgagagtgaaatctgtccgagatccactgccactgaaccttgatgggaccccagagtttaggttggatgcaagatagatcaggagcttaggggctttccctggtttctgctgataccagtgcagaaaactattgccataaccatcaacactttcactggctctgcaagtgatggtgactctgtctcctacagatgcagacagggaggatggagactgcgtcaacactatttc

SEQ ID NO: 189 - DNA, 30 bp, Artificial Sequence
  Synthetic construct
aataaacttgagttcatgggatacataagt

SEQ ID NO: 190 - DNA, 30 bp, Artificial Sequence
  Synthetic construct
acttatgtatcccatgaactcaagtttatt

SEQ ID NO: 191 - DNA, 30 bp, Artificial Sequence
  Synthetic construct
gaataaacttgagtggatgggatacataag

SEQ ID NO: 192 - DNA, 30 bp, Artificial Sequence
  Synthetic construct
cttatgtatcccatccactcaagtttattc

SEQ ID NO: 193 - DNA, 405 bp, Artificial Sequence
  Synthetic construct
atgaaacatctgtggttcttccttctgctggtggcagctcccagatgggtcctgtcccaggtgcagctgcaggagtcgggcccaggactggtgaagccttcggagaccctgtccctcacctgcactgtctctggtgactccatcagtagtggttactggaacatccggcagcccccagggagggcactggagtggataggatacataagttacagtggtagcacttactacaatctatctctcagaagtcgggtcaccatatcagtagacacgtctaagaaccagttctccctgaggctgagctctgtgaccgctgcggacacggccatgtattactgtgcgagaattactacgactacctatgctatggactactggggccaagggaccacggtcaccgtctcc

SEQ ID NO: 194 - DNA, 60 bp, Artificial Sequence
  Synthetic construct
atggtgttgcagacccaggtcttcatttctctgttgctctggatctctggggcttacggg

SEQ ID NO: 195 - protein, 20 aa, Artificial Sequence
  Synthetic construct
MVLQTQVFISLLLWISGAYG

SEQ ID NO: 196 - DNA, 66 bp, Artificial Sequence
  Synthetic construct
atggacatgagggtccccgctcagctcctggggctcctgctgctctggctcccaggcgccagatgt

SEQ ID NO: 197 - protein, 22 aa, Artificial Sequence
  Synthetic construct
MDMRVPAQLLGLLLLWLPGARC

SEQ ID NO: 198 - protein, 16 aa, Artificial Sequence
  Synthetic construct
QLINTNGSWHINGSGK

SEQ ID NO: 199 - protein, 16 aa, Artificial Sequence
  Synthetic construct
NLINTNGSWHINGSGK

SEQ ID NO: 200 - protein, 16 aa, Artificial Sequence
  Synthetic construct
QLINTNGSWHVNGSGK

SEQ ID NO: 201 - protein, 16 aa, Artificial Sequence
  Synthetic construct
QLINSNGSWHINGSGK

SEQ ID NO: 202 - protein, 16 aa, Artificial Sequence
  Synthetic construct
QLVNSNGSWHINGSGK

SEQ ID NO: 203 - protein, 16 aa, Artificial Sequence
  Synthetic construct
VELRNLGGTWRPGSGK

SEQ ID NO: 204 - DNA, 20 bp, Artificial Sequence
  Synthetic construct
ctgcggaaccggtgagtaca

SEQ ID NO: 205 - DNA, 21 bp, Artificial Sequence
  Synthetic construct
tgcacggtctacgagacctcc

SEQ ID NO: 206 - DNA, 22 bp, Artificial Sequence
  Synthetic construct
acccggtcgtcctggcaattcc

SEQ ID NO: 207 - DNA, 20 bp, Artificial Sequence
  Synthetic construct
cttcacgcagaaagcgccta

SEQ ID NO: 208 - DNA, 20 bp, Artificial Sequence
  Synthetic construct
caagcaccctatcaggcagt

SEQ ID NO: 209 - DNA, 20 bp, Artificial Sequence
  Synthetic construct
tatgagtgtcgtacagcctc

SEQ ID NO: 210 - DNA, 19 bp, Artificial Sequence
  Synthetic construct
gaaggtgaaggtcggagtc

SEQ ID NO: 211 - DNA, 20 bp, Artificial Sequence
  Synthetic construct
gaagatggtgatgggatttc

SEQ ID NO: 212 - DNA, 20 bp, Artificial Sequence
  Synthetic construct
atgaccccttcattgacctc

(19) United States
(12) Patent Application Publication
(10) Pub. No.: US 2012/0027722 A1
(43) Pub. Date: Feb. 2, 2012

(54) HEPATITIS C VIRUS COMBINATION THERAPY

(75) Inventors: Sharookh Kapadia, San Jose, CA (US); Arvind Patel, Glasgow (GB); David J. Matthews, London (GB); David G. Williams, London (GB)

(73) Assignee: Medical Research Council

(21) Appl. No.: 13/140,000

(22) PCT Filed: Dec. 17, 2009
(86) PCT No.: PCT/US09/68556

§ 371 Date: Oct. 7, 2011

Related U.S. Application Data
(60) Provisional application No. 61/138,478, filed on Dec. 17, 2008.

(51) Int. Cl. A61K 38/21; A61P 37/04; A61P 31/14

(52) U.S. Cl. 424/85.7

(57) ABSTRACT

The present invention relates to methods and compositions for the treatment or prevention of hepatitis C virus comprising the administration of a combination of anti-hepatitis C virus antibodies and a-interferon.

### CROSS-REFERENCE TO RELATED APPLICATIONS

This application claims priority benefit to provisional application 61/138,478, filed on Dec. 17, 2008, the contents of which are incorporated by reference herein in their entirety.

### FIELD OF THE INVENTION

The present invention relates to methods and compositions for the treatment or prevention of hepatitis C virus comprising the administration of a combination of anti-hepatitis C virus antibodies and a-interferon.

### BACKGROUND OF THE INVENTION

HCV is a positive strand RNA virus belonging to the Flaviviridae family. It is the major cause of non-A non-B viral hepatitis. HCV has infected approximately 200 million people and current estimates suggest that as many as 3 million individuals are newly infected each year. Approximately 80% of those infected fail to clear the virus; a chronic infection ensues, frequently leading to severe chronic liver disease, cirrhosis and hepatocellular carcinoma. Current treatments for chronic infection are ineffective and there is a pressing need to develop preventative and therapeutic vaccines.

Due to the error-prone nature of the RNA-dependent RNA polymerase and the high replicative rate in vivo, HCV exhibits a high degree of genetic variability. HCV can be classified into six genetically distinct genotypes and further subdivided into at least 70 subtypes, which differ by approximately 30% and 15% at the nucleotide level, respectively. A significant challenge for the development of vaccines will be identifying protective epitopes that are conserved in the majority of viral genotypes and subtypes. This problem is compounded by the fact that the envelope proteins, the natural target for the neutralizing response, are two of the most variable proteins.

The envelope proteins, E1 and E2, are responsible for cell binding and entry. They are N-linked glycosylated transmembrane proteins with an N-terminal ectodomain and a C-terminal hydrophobic membrane anchor. In vitro expression experiments have shown that E1 and E2 proteins form a non-covalent heterodimer, which is proposed to be the functional complex on the virus surface. Due to the lack of an efficient culture system, the exact mechanism of viral entry is unknown. That said, there is mounting evidence that entry into isolated primary liver cells and cell lines requires interaction with the cell surface receptors CD81 and Scavenger Receptor Class B Type 1 (SR-B1), although these receptors alone are not sufficient to allow viral entry.

Current evidence suggests that cell mediated immunity is pivotal in clearance and control of viral replication in acute infection. However, surrogate models of infection, such as animal infection and cell and receptor binding assays, have highlighted the potential role of antibodies in both acute and chronic infection. Unsurprisingly, neutralizing antibodies recognize both linear and conformational epitopes. The majority of antibodies that demonstrate broad neutralization capacity are directed against conformational epitopes within E2. Induction of antibodies recognizing conserved conformational epitopes is extremely relevant to vaccine design, but this is likely to prove difficult, as the variable regions appear to be immuno-dominant. One such immuno-dominant linear epitope lies within the first hypervariable region of E2 (HVR1). The use of conserved HVR1 mimotopes has been proposed to overcome problems of restricted specificity, but it is not yet known whether this approach will be successful.

A region immediately downstream of HVR1 contains a number of epitopes. One epitope, encompassing residues 412-423 and defined by the monoclonal antibody AP33, inhibits the interaction between CD81 and a range of presentations of E2, including soluble E2, E1E2 and virus-like particles. See Owsianka A. et al., J Gen Virol 82:1877-83 (2001).

WO 2006/100449 teaches that the monoclonal antibody designated AP33 can bind to and neutralize each of the six known genotypes 1-6 of HCV. Accordingly, it is deduced that the epitope targeted by AP33 is cross-reactive with all of genotypes 1-6 of HCV, indicating it as a target for anti-HCV ligands and as an immunogen for raising anti-HCV antibodies.

More effective treatments for hepatitis C virus are needed.

The disclosures of all publications, patents, patent applications and published patent applications referred to herein are hereby incorporated herein by reference in their entirety.

### BRIEF SUMMARY OF THE INVENTION

The present invention relates to methods and compositions for the treatment or prevention of hepatitis C virus comprising the administration of a combination of anti-hepatitis C virus antibodies and a-interferon.

Provided herein are methods of treating or preventing a HCV infection in a subject, comprising administering to the individual: a) an effective amount of a composition comprising an anti-HCV antibody that binds hepatitis E2 protein; and b) an effective amount of a-interferon.

In some embodiments, the anti-HCV antibody is a monoclonal antibody.

In some embodiments, the monoclonal antibody comprises (a) a light chain variable domain comprising (i) CDR-L1 comprising sequence RASESVDGYGNSFLH (SEQ ID NO:41); (ii) CDR-L2 comprising sequence LASNLNS (SEQ ID NO:42); and (iii) CDR-L3 comprising sequence QQNNVDPWT (SEQ ID NO:43) and (b) a heavy chain variable domain comprising (i) CDR-H1 comprising sequence GDSITSGYWN (SEQ ID NO:44); (ii) CDR-H2 comprising sequence YISYSGSTY (SEQ ID NO:45); and (iii) CDR-H3 comprising sequence ITTTTYAMDY (SEQ ID NO:46).

In some embodiments, the monoclonal antibody comprises (a) a light chain variable domain comprising (i) CDR-L1 comprising sequence RASESVDGYGNSFLH (SEQ ID NO:41); (ii) CDR-L2 comprising sequence LASNLNS (SEQ ID NO:42); and (iii) CDR-L3 comprising sequence QQNNVDPWT (SEQ ID NO:43) and (b) a heavy chain variable domain comprising (i) CDR-H1 comprising sequence SGYWN (SEQ ID NO:47); (ii) CDR-H2 comprising sequence YISYSGSTYYNLSLRS (SEQ ID NO:48); and (iii) CDR-H3 comprising sequence ITTTTYAMDY (SEQ ID NO:46).

In some embodiments, the monoclonal antibody is a humanized antibody.

In some embodiments, the humanized antibody comprises (a) a light chain variable domain comprising (i) CDR-L1 comprising sequence RASESVDGYGNSFLH (SEQ ID NO:41); (ii) CDR-L2 comprising sequence LASNLNS (SEQ ID NO:42); and (iii) CDR-L3 comprising sequence QQNNVDPWT (SEQ ID NO:43) and (b) a heavy chain variable domain comprising (i) CDR-H1 comprising sequence GDSITSGYWN(SEQ ID NO:44); (ii) CDR-H2 comprising sequence YISYSGSTY (SEQ ID NO:45); and (iii) CDR-H3 comprising sequence ITTTTYAMDY (SEQ ID NO:46).

In some embodiments, the humanized antibody comprises (a) a light chain variable domain comprising (i) CDR-L1 comprising sequence RASESVDGYGNSFLH (SEQ ID NO:41); (ii) CDR-L2 comprising sequence LASNLNS (SEQ ID NO:42); and (iii) CDR-L3 comprising sequence QQNNVDPWT (SEQ ID NO:43) and (b) a heavy chain variable domain comprising (i) CDR-H1 comprising sequence SGYWN (SEQ ID NO:47); (ii) CDR-H2 comprising sequence YISYSGSTYYNLSLRS (SEQ ID NO:48); and (iii) CDR-H3 comprising sequence ITTTTYAMDY(SEQ ID NO:46).

In some embodiments, the humanized antibody comprises a variable heavy chain domain selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18 and a variable light chain domain selected from the group consisting of SEQ ID NO:6, SEQ ID NO:7, SEQ ID NO:19, and SEQ ID NO:20.

In some embodiments of any of the methods, the antibody is an antigen binding fragment. In some embodiments, the antigen binding fragment is selected from the group consisting of a Fab fragment, a Fab′ fragment, a F(ab′)₂ fragment, a scFv, a Fv, and a diabody.

In some embodiments of any of the methods, the a-interferon is selected from a group consisting of IFN-a1, IFN-a2, IFN-a4, IFN-a5, IFN-a6, IFN-a7, IFN-a8, IFN-a10, IFN-a13, IFN-a 14, IFN-a 16, IFN-a 17, and IFN-a21. In some embodiments, the a-interferon is IFN-a2. In some embodiments, the IFN-a2 is selected from the group consisting of IFN-a2a, IFN-a2b, or IFN-a2c. In some embodiments, the IFN-a2 is pegylated.

In some embodiments of any of the methods, the anti-HCV antibody is administered simultaneously, concurrently, rotationally, intermittently, or sequentially with a-interferon.

In some embodiments of any of the methods, the hepatitis C virus infection is an acute hepatitis C virus infection.

In some embodiments of any of the methods, the hepatitis C virus infection is a chronic hepatitis C virus infection.

In some embodiments of any of the methods, the method comprises treat the hepatitis C virus infection. In some embodiments, treating the hepatitis C virus infection comprises reducing viral load. In some embodiments, treating the hepatitis C virus infection comprises reducing viral titer.

### BRIEF DESCRIPTION OF THE DRAWINGS

FIGS. 1A-1B show AP33RHA protein and DNA sequence generation. FIG. 1A shows AP33RHA protein sequence graft (SEQ ID NOS:1 and 46-54), and FIG. 1B shows AP33RHA DNA sequence graft (SEQ ID NOS:46-48, 50-53, and 55-63). Dark grey highlighting with bolded text indicates CDRs.

FIGS. 2A-2B show AP33RHA leader selection and SignalP results with VH4-59 leader and S67826 FW1 [14] (SEQ ID NO:55).

FIG. 3 shows DNA (SEQ ID NO:64) and Protein sequence (SEQ ID NO:65) of AP33RHA. Light grey boxes show nucleotide changes that remove cryptic splice sites.

FIGS. 4A and 4B show generation of AP33RKA sequence. FIG. 4A shows AP33RKA protein sequence graft (SEQ ID NOS:2, 4, 41-43, and 66-70). FIG. 4B shows AP33RKA DNA sequence graft (SEQ ID NOS:41-43, 66-69, and 71-78) with VKIV B3 leader. CDRs are highlighted.

FIGS. 5A-5B show generation of AP33RK2 sequence. FIG. 5A shows AP33RK2 protein sequence graft (SEQ ID NOS:2, 41-43, and 79-84). FIG. 5B shows AP33RK2 DNA sequence generation (SEQ ID NOS:41-43, 69, 71, 72, 74, 75, 77, 80-82, and 85-88). CDRs are highlighted.

FIGS. 6A-6D show AP33RKA (SEQ ID NO:89), AP33RK2 (SEQ ID NO:90), AP33RK3 (SEQ ID NO:91), and AP33RK4 (SEQ ID NO:92) DNA sequence with leader. CDRs are highlighted.

FIG. 7 shows DNA (SEQ ID NO:93) and protein sequence (SEQ ID NO:94) of AP33RKA with leader. Light grey boxes represent changed nucleotides to remove cryptic splice sites or unwanted BamHI sites.

FIG. 8 shows DNA (SEQ ID NOS:95 and 96 (complementary sequence)) and protein sequence (SEQ ID NO:97) of AP33RK2 with leader. Light grey boxes represent changed nucleotides to remove cryptic splice sites or unwanted BamHI sites.

FIG. 9 shows VCI of AP33 VK and non-VK4 human VK sequences with longer CDR1 (SEQ ID NOS:98-103). Twenty human VK nonVK4 sequences with best VCI scores, matching CDR2 size and longer CDR1 compared to AP33VK. VCI/FW score indicates number of VCI or FW residues identical to AP33VK.

FIG. 10 shows AP33 VK (SEQ ID NO:2) and human VK non-VK4 sequences with larger CDR1 (SEQ ID NOS:104-123). Cys, Pro and CDRs are indicated by dark grey highlighting with bolded text, black highlighting with white text, and light grey highlighting, respectively.

FIG. 11 shows ClustalW alignment of AP33 VK (SEQ ID NO:2) and non VK4 human sequences (SEQ ID NOS:124-141) with larger CDR1. Residues identical to AP33VK are indicated by a dot. In the top 7 sequences, conservative changes are medium grey and non conservative changes are dark grey. CDRs are light grey.

FIG. 12 shows predicted signal protease cleavage result [22] with VKII-A17 leader and AB064133 FW1.

FIG. 13 (previously Table 7 in 61/006,066) shows Comparison of Vernier Canonical and Interface residues in AP33 H (SEQ ID NO:1) and L chains (SEQ ID NO:2) with the donor sequences. ‘Vern/CDR’ indicates vernier residues (v) and CDRs (-===-). Light grey highlighting indicates CDRs. Black highlighting with white text indicates VCI residues. Dark gray highlighting with bolded text differences between the VCI residues found in AP33 and S67826 (SEQ ID NO:142), X61125 (SEQ ID NO:70), AB064133 (SEQ ID NO:144), AB064072 (SEQ ID NO:145) and AY68527 (SEQ ID NO:143).

FIGS. 14A-14B show generation of AP33RK3 sequence. FIG. 14A shows AP33RK3 protein sequence graft (SEQ ID NOS: 2, 41-43, 144, and 146-150). FIG. 14B shows AP33RK3 DNA sequence generation (SEQ ID NOS: 41-43, 74, 75, 77, and 147-156). CDRs are highlighted.

FIG. 15 shows AP33RK4 DNA sequence generation. CDRs are highlighted (SEQ ID NOS:41-43, 72, 74, 75, 77, 83, 88, and 157-163).

FIG. 16 shows DNA (SEQ ID NO:164) and protein (SEQ ID NO:165) sequence of AP33RK3 construct. NB AP33RLB has the two VCIs back mutated. No splice sites are generated by this.

FIG. 17 shows DNA (SEQ ID NOS:166 and 167 (complementary sequence)) and protein sequence (SEQ ID NO:168) of AP33RK4 construct.

FIG. 18 shows the binding of humanized and chimeric antibody to the AP33 mimotope H6. To compare relative binding of the chimeric antibody to the humanized heavy and light chains COS7 cells were transfected with a series of chimeric and humanized heavy and light chain constructs and the supernatants were used to compare binding to the mimotope H6. The binding of the mimotope peptide H6 to chimeric (Vh/Vl) and humanized antibodies RHb-h/RK2bc and RHA/RK2bc or mixtures of humanized and chimeric antibodies RHA/Vl, RHb-h/Vl or Vh/RK2bc were measured by ELISA.

FIG. 19 shows the binding of AP33RHI to peptide H6. To determine if the heavy chain interface residue Q39 is responsible for the suboptimal binding to peptide H6 the binding of humanized heavy chain RHI (Q39K) was measured by ELISA. COS7 cells were transfected with a series of chimeric and RHI heavy and RK2b light chain constructs and the supernatants were used to compare binding to the mimotope H6. The binding of the mimotope peptide H6 to chimeric (Vh/Vl) and humanized antibodies RHb-h, RHI/RK2bc and mixtures of humanized and chimeric antibodies RHI/Vl or RHb-h/Vl were measured by ELISA.

FIGS. 20A-20B show the binding of Chimeric and humanized antibody to E2 peptides. To compare relative binding of the chimeric antibody to the humanized antibody RHb-h/Vl, COS7 cells were transfected with a series of chimeric and humanized antibody constructs. The antibody supernatants were subjected to ELISA and used to compare binding to the peptides described in Table 5.

FIG. 21 shows the binding of RK2 variants to H6 peptide. The minimal number of mutations necessary for the humanized light chain RK2 to function was determined by comparing the binding of RHb-g with the light chains RK2, RK2b, and RK2c. The chimeric antibody Vh/Vl was included as a comparator to previous experiments. COS7 cells were transfected with a series of chimeric and humanized antibody constructs. The binding of antibody supernatants to the E2 peptides (Table 5) were measured by ELISA.

FIGS. 22A-22I show the binding of E2 peptides to the humanized heavy chain VC variants. The minimal number of mutations necessary for the humanized heavy chain RHb-h to function was determined by comparing the binding of RHb-h with the back mutated heavy chains RH-B, RH-C, RH-D, RH-E, RH-F, RH-G and RH-H. The chimeric antibody Vh/Vl was included as a comparator to previous experiments and the humanized light chain RK2bc was used to pair with all the humanized heavy chains. COS7 cells were transfected with a series of chimeric and humanized antibody constructs. The binding of antibody supernatants to the E2 peptides (Table 5) were measured by ELISA.

FIGS. 23A-23E shows a comparison of humanized antibody VC mutants binding to E2 peptides using normalized data. The data from FIG. 22 was analyzed further by normalizing each data set as a percentage of H6 binding and each genotype grouped together.

FIGS. 24A-24E show that humanized AP33 antibodies inhibit HCVpp infection. Neutralization by chimeric AP33 or humanized antibodies of HCVpp derived from diverse genotypes. HCVpp were preincubated for 1 hour at 37° C. with different concentrations of purified chimeric AP33 or humanized antibodies prior to infection of Huh-7 cells. The neutralizing activity of the antibody is expressed as percentage of inhibition of the infectious titers.

FIGS. 25A-25B show the neutralization by chimeric AP33 or humanized antibodies of HCVpp derived from genotype 5. HCVpp were pre-incubated for 1 hour at 37° C. with different concentrations of purified chimeric AP33 or humanized antibodies prior to infection of Huh-7 cells. The neutralizing activity of the antibody is expressed as percentage of inhibition of the infectious titers. Results from two separate experiments are shown.

FIGS. 26A-26E show the binding of AP33 mutants Y47F and Y47W to E2 peptides. The impact of mutating residue Y47 of the chimeric heavy of AP33. The binding of the E2 peptides shown in Table 5 were used to compare wild type heavy chain AP33 and the mutants Y47F and Y47W. COS7 cells were transfected with a series of chimeric and mutant antibody constructs. The binding of antibody supernatants to the E2 peptides were measured by ELISA. The data was manipulated by normalizing each data set as a percentage of H6 binding and each genotype grouped together.

FIG. 27 shows inhibition of HCVpp infection by AP33 mutants Y47F and Y47W. Neutralization by chimeric AP33 or humanized antibodies of HCVpp derived from 1a genotype. HCVpp were preincubated for 1 hour at 37° C. with different concentrations of purified chimeric AP33 or humanized antibodies prior to infection of Huh-7 cells. The neutralizing activity of the antibody is expressed as percentage of inhibition of the infectious titers.

FIGS. 28A-28C. FIG. 28A shows AP33 and RH-C/RK2b neutralization of Con1 HCVpp as measured by percent infection. FIG. 28B shows AP33 and RH-C/RK2b neutralization of J6 HCVpp as measured by percent infection. FIG. 28C shows the EC₅₀ (μg/ml) of AP33 and RH-C/RK2b using Con1 and J6 HCVpp.

FIGS. 29A-29C. FIG. 29A shows AP33 and RH-C/RK2b neutralization of Con1 HCVcc as measured by percent infection. FIG. 29B shows AP33 and RH-C/RK2b neutralization of J6 HCVcc as measured by percent infection. FIG. 29C shows the EC₅₀ (μg/ml) of AP33 and RH-C/RK2b using Con1 and J6 HCVcc.

FIGS. 30A-30B. FIG. 30A shows the results of a neutralization assay using Con1 HCVpp in the presence of RH-C/RK2b and 10% normal human serum (NHS) or sera from chronic HCV-infected patients (CHCHS-1 and CHCHC-2). FIG. 30B shows level of binding of NHS, CHCHS-1, CHCHS-2, and RH-C/RK2b to Con1 HCV E1E2-reactive antibodies by ELISA assay using lysates from GT1b (Con1) E1E2-transfected 293T cells as measured by absorbance (A450).

FIG. 31A-31B shows HCV RNA as measured at day 14 or 18 post infection to analyze infection when treated with RH-C/RK2b or IFN-a alone or the combination of RH-C/RK2b plus IFN-a. FIG. 31A shows HCV RNA levels on day 14 post infection. FIG. 31B shows HCV RNA levels on day 18 post infection.

FIGS. 32A1-32G3 shows the amino acid and nucleotide sequences (SEQ ID NOS:1-40 and 169-188) of humanized antibody variable chains of Table 6.

### DETAILED DESCRIPTION OF THE INVENTION

### I. Therapeutic Uses

The present invention provides methods of combination therapy comprising a first therapy comprising an antibody which binds hepatitis C virus “HCV” in conjunction with a-interferon. The combination of anti-HCV antibodies and a-interferon can be used for treating a HCV infection. The combination of anti-HCV antibodies and a-interferon are useful in reducing, eliminating, or inhibiting HCV infection and can be used for treating any pathological condition that is characterized, at least in part, by HCV infection.

The term “hepatitis C virus” or “HCV” is well understood in the art and refers to a virus which is a member of the genus Hepacivirus of the family flaviviridae. HCV is a lipid enveloped virus having a diameter of approximately 55-65 nm in diameter with a positive strand RNA genome. The hepatitis C virus species is classified into six genotypes (1-6) with several subtypes within each genotype. In some embodiments, the subject is infected with one or more HCV genotypes selected from the group consisting of genotype 1 (e.g., genotype 1a and genotype 1b), genotype 2 (e.g., genotype 2a, genotype 2b, genotype 2c), genotype 3 (e.g., genotype 3a), genotype 4, genotype 5, and genotype 6. In North America, genotype 1a predominates followed by 1b, 2a, 2b, and 3a. In Europe, genotype 1b is predominant followed by 2a, 2b, 2c, and 3a. Genotypes 4 and 5 are found almost exclusively in Africa.

Provided herein are methods of treating a HCV infection in a subject, comprising administering to the individual: a) an effective amount of a composition comprising an anti-HCV antibody; and b) an effective amount of a-interferon.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life.

In some embodiments, the combination of anti-HCV antibodies and a-interferon are useful in methods of treating an acute HCV infection. In some embodiments, treating an acute HCV infection includes reducing, eliminating, or inhibiting an acute HCV infection.

The term “acute hepatitis C virus infection” or “acute HCV infection,” as used herein, refers to the first 6 months after infection with HCV.

In some embodiments, a subject with an acute HCV infection will not develop any symptoms (i.e., free of acute HCV infection symptoms). Between 60% to 70% of subjects with acute HCV infection develop no symptoms during the acute phase. In some embodiments, a subject with acute HCV infection will develop symptoms. In some embodiments, the methods of treatment described herein ameliorate (e.g., reduce incidence of, reduce duration of, reduce or lessen severity of) of one or more symptoms of acute HCV infection. In the minority of patients who experience acute phase symptoms, the symptoms are generally mild and nonspecific, and rarely lead to a specific diagnosis of hepatitis C. Symptoms of acute hepatitis C infection include decreased appetite, fatigue, abdominal pain, jaundice, itching, and flu-like symptoms. In some embodiments, the subject with acute HCV infection is infected with HCV of the genotype 1. Treatment during the acute HCV injection of genotype 1 has a greater than 90% success rate with half the treatment time required for chronic infections.

In some embodiments, the combination of anti-HCV antibodies and a-interferon are useful in methods of treating a chronic HCV infection. In some embodiments, treating a chronic HCV infection includes reducing, eliminating, or inhibiting a chronic HCV infection.

The term “chronic hepatitis C virus infection” or “chronic HCV infection,” as used herein, refers to as infection with HCV which persisting for more than six months.

In some embodiments, the methods of treatment described herein ameliorate (e.g., reduce incidence of, reduce duration of, reduce or lessen severity of) of one or more symptoms of chronic HCV infection. Symptoms of chronic HCV infection include fatigue, marked weight loss, flu-like symptoms, muscle pain, joint pain, intermittent low-grade fevers, itching, sleep disturbances, abdominal pain (especially in the right upper quadrant), appetite changes, nausea, diarrhea, dyspepsia, cognitive changes, depression, headaches, and mood swings. Once chronic HCV has progressed to cirrhosis, signs and symptoms may appear that are generally caused by either decreased liver function or increased pressure in the liver circulation, a condition known as portal hypertension. Possible signs and symptoms of liver cirrhosis include ascites, bruising and bleeding tendency, bone pain, varices (especially in the stomach and esophagus), fatty stools (steatorrhea), jaundice, and a syndrome of cognitive impairment known as hepatic encephalopathy. In some embodiments, the chronic HCV infection may result in hepatocellular carcinoma (HCC). Chronic HCV infection can be further divided into two types (either or both of which are included in the methods of treatment provided herein) chronic active HCV infection and chronic persistent HCV infection. Chronic active HCV infection is HCV which is cause active damage to the liver. Chronic persistent HCV infection is a chronic HCV infection which is not currently causing damage to the liver, although pre-existing liver damage may be present.

In some embodiments, the humanized antibodies may be administered to the subject infected with HCV prior to, concurrent with, or subsequent to a liver transplant.

In some embodiments of any of the methods of treating, the combination of anti-HCV antibodies and a-interferon are useful in methods of treatment including suppressing one or more aspects of a HCV infection. In some embodiments, the HCV infection is a chronic HCV infection. In some embodiments, the HCV infection is an acute HCV infection. In some embodiments, the methods described herein suppress a HCV-associated laboratory finding (e.g., ALAT, AST, and GGTP levels in blood), viral replication, viral titer, viral load, or viremia.

In some embodiments, the methods described herein suppress or reduce viral titer. “Viral titer” is known in the art and indicates the amount of virus in a given biological sample.

In some embodiments, the methods described herein suppress or reduce viremia. “Viremia” is known in the art as the presence of virus in the bloodstream and/or viral titer in a blood or serum sample.

In some embodiments, the methods described herein suppress or reduce viral load. “Viral load” refers to the amount of hepatitis C virus in a person's blood. The results of a hepatitis C viral load test (known as a viral RNA test or HCV RNA test) are usually expressed as International Units/mL (IU/mL) or RNA copies/mL. A subject with a hepatitis C viral load of 1 million IU/mL or more is considered to have a high viral load.

Amount of virus (e.g., viral titer or viral load) are indicated by various measurements, including, but not limited to amount of viral nucleic acid, the presence of viral particles, replicating units (RU), plaque forming units (PFU). Generally, for fluid samples such as blood and urine, amount of virus is determined per unit fluid, such as milliliters. For solid samples, such as tissue samples, amount of virus is determined per weight unit, such as grams. Methods for determining amount of virus are known in the art and are also described herein.

In some embodiments, the subject treated with combination of anti-HCV antibodies and a-interferon is at risk for rapid HCV infection progression. Factors that have been reported to influence the rate of HCV disease progression include age (increasing age associated with more rapid progression), gender (males have more rapid disease progression than females), alcohol consumption (associated with an increased rate of disease progression), HIV co-infection (associated with a markedly increased rate of disease progression), and fatty liver (the presence of fat in liver cells has been associated with an increased rate of disease progression).

In some embodiments of any of the methods, the subject produces anti-HCV antibodies (i.e., endogenous anti-HCV antibodies). In some embodiments, the anti-HCV antibodies are detectable, e.g., the anti-HCV antibodies are detectable by ELISA. In some embodiments, the anti-HCV antibodies produced by the subject are neutralizing antibodies. In some embodiments, the anti-HCV antibodies produced by the subject are non-neutralizing antibodies.

Provided herein are methods of preventing a HCV infection in a subject, comprising administering to the individual: a) an effective amount of a composition comprising an anti-HCV antibody; and b) an effective amount of a-interferon. In some embodiments, the anti-HCV antibodies and a-interferon can be used in methods for preventing a HCV infection in a subject susceptible to infection with HCV. In some embodiments, the combination of anti-HCV antibodies and a-interferon can also be used in methods for preventing a HCV infection in a subject exposed to or potentially exposed to HCV. “Exposure” to HCV denotes an encounter or potential encounter with HCV which could result in an HCV infection. Generally, an exposed subject is a subject that has been exposed to HCV by a route by which HCV can be transmitted. In some embodiments, the subject has been exposed to or potentially exposed to blood of a subject with an HCV infection or blood from a subject which may or may not be infected with HCV (i.e., HCV infection status of the blood exposure is unknown). HCV is often transmitted by blood-to-blood contact. In some embodiments, the subject has been exposed to or potentially exposed to HCV by, but not limited to, use of blood products (e.g., a blood transfusion), “needle stick” accidents, sharing drug needles, snorting drugs, a sexual partner, iatrogenic medical or dental exposure, needles used in body piercings and tattoos, or a child whose mother has an HCV infection. In some embodiments of the methods of prevention, the anti-HCV antibodies and a-interferon described herein will be administered at the time or within any of about one day, one week, or one month of the exposure or potential exposure to HCV.

In some embodiments of any of the methods described herein, the subject is a human or chimpanzee. In some embodiments, the subject is a human. HCV infects only human and chimpanzee.

In some embodiments of any of the methods described herein, the method comprises administering the anti-HCV antibody in combination with, sequential to, concurrently with, consecutively with, rotationally with, or intermittently with a-interferon. In some embodiment, the method comprising administering the combination of the antibody which binds HCV and a-interferon ameliorates one or more symptom of HCV, reduces and/or suppresses viral titer and/or viral load, and/or prevents HCV more than treatment with the anti-HCV antibody or a-interferon alone. In some embodiment, the combination of anti-HCV antibodies and a-interferon ameliorates one or more symptom of HCV, reduces and/or suppresses viral titer and/or viral load, and/or prevents HCV about any of 25%, 50%, 75%, 100%, 150%, 200%, 250%, 300%, 350%, 400%, 450%, or 500% more than the anti-HCV antibody or a-interferon alone.

In some embodiments of any of the methods described herein, the anti-HCV antibody binds to HCV. In some embodiments, the antibody is capable of binding to HCV E2 protein, soluble HCV E2 protein, or a heterodimer of HCV E1 protein and HCV E2 protein. In some embodiments, the anti-HCV antibody binds HCV E2 protein. In some embodiments, the HCV E2 protein is from one or more of the HCV genotypes selected from the group consisting of genotype 1 (e.g., genotype 1a and genotype 1b), genotype 2 (e.g., genotype 2a, genotype 2b, genotype 2c), genotype 3 (e.g., genotype 3a), genotype 4, genotype 5, and genotype 6. In some embodiments, the anti-HCV antibody inhibits the interaction of HCV E2 protein with CD81. In some embodiments, the anti-HCV antibody prevents and/or inhibits HCV entry into the cell. In some embodiments, the cell is a liver cell, e.g., hepatocyte. In some embodiments, the anti-HCV antibody is any antibody described herein. In some embodiments, the anti-HCV antibody is an antibody fragment.

Reference to “about” a value or parameter herein includes (and describes) variations that are directed to that value or parameter per se. For example, description referring to “about X” includes description of “X”.’

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### II. Antibodies

The methods of combination therapy provided herein use, or incorporate, anti-HCV antibodies. In some embodiments, the anti-HCV antibodies bind to HCV. In some embodiments, the anti-HCV antibodies bind to HCV E2 protein. Accordingly, methods for generating such antibodies will be described here. A description follows as to exemplary techniques for the production of the antibodies used in methods provided herein.

The antibodies may be characterized in a number of ways which will be apparent to those skilled in the art. These include physical measurements of their concentration by techniques such as ELISA, and of the antibody purity by SDS-PAGE. In addition the efficacy of the polypeptides can be determined by detecting the binding of the molecule to HCV E2 glycoprotein in solution or in a solid phase system such as ELISA, surface plasmon resonance (e.g., BIAcore) or immunofluorescence assays. More especially, the neutralizing capability of the polypeptide can be tested against HCV samples representative of the six known genotypes in a HCV pp-neutralizing assay as described herein, such as HCVpp and HCVcc neutralization assays.

### (i) Definitions

Antibodies are naturally occurring immunoglobulin molecules which have varying structures, all based upon the immunoglobulin fold. For example, IgG antibodies such as AP33 have two ‘heavy’ chains and two ‘light’ chains that are disulphide-bonded to form a functional antibody. Each heavy and light chain itself comprises a “constant” (C) and a “variable” (V) region. The V regions determine the antigen binding specificity of the antibody, whilst the C regions provide structural support and function in non-antigen-specific interactions with immune effectors. The antigen binding specificity of an antibody or antigen-binding fragment of an antibody is the ability of an antibody to specifically bind to a particular antigen.

The antigen binding specificity of an antibody is determined by the structural characteristics of the V region. The variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

In some embodiments, the hypervariable regions are the amino acid residues of an antibody which are responsible for antigen-binding. The hypervariable region may comprise amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

Each V region typically comprises three complementarity determining regions (“CDRs”, each of which contains a “hypervariable loop”), and four framework regions. An antibody binding site, the minimal structural unit required to bind with substantial affinity to a particular desired antigen, will therefore typically include the three CDRs, and at least three, preferably four, framework regions interspersed there between to hold and present the CDRs in the appropriate conformation. Classical four chain antibodies, such as AP33, have antigen binding sites which are defined by V_{H} and V_{L} domains in cooperation. Certain antibodies, such as camel and shark antibodies, lack light chains and rely on binding sites formed by heavy chains only. Single domain engineered immunoglobulins can be prepared in which the binding sites are formed by heavy chains or light chains alone, in absence of cooperation between V_{H} and V_{L}.

Throughout the present specification and claims, unless otherwise indicated, the numbering of the residues in the constant domains of an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody. The residues in the V region are numbered according to Kabat numbering unless sequential or other numbering system is specifically indicated.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

For the purposes herein, an “intact antibody” is one comprising heavy and light variable domains as well as an Fc region.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called a, d, e, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains that enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the methods provided herein may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody that are responsible for antigen binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

A “naked antibody” is an antibody (as herein defined) that is not conjugated to a heterologous molecule, such as a cytotoxic moiety or radiolabel.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In some embodiments, the antibody will be purified (1) to greater than 95% by weight of antibody as determined by the Lowry method, and in some embodiments, more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, in some embodiments, silver stain. Isolated antibody includes the antibody in situ within recombinant cells since at least one component of the antibody's natural environment will not be present. Ordinarily, however, isolated antibody will be prepared by at least one purification step. The antibody or antibody fragment described herein may be isolated or purified to any degree. As used herein, “isolated” means that that antibody or antibody fragment has been removed from its natural environment. In some embodiments, contaminant components of its natural environment are materials which would interfere with diagnostic or therapeutic uses for the antibody, and may include enzymes, hormones, and other proteinaceous or nonproteinaceous solutes. In some embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“Purified” means that the antibody or antibody fragment has been increased in purity, such that it exists in a form that is more pure than it exists in its natural environment and/or when initially synthesized and/or amplified under laboratory conditions. Purity is a relative term and does not necessarily mean absolute purity.

In some embodiments, antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes that express one or more FcRs and perform effector functions. In some embodiments, the cells express at least FcγRIII and carry out ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. In some embodiments, the FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement dependent cytotoxicity” or “CDC” refer to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

### (ii) Polyclonal Antibodies

Polyclonal antibodies are preferably raised in animals by multiple subcutaneous (sc) or intraperitoneal (ip) injections of the relevant antigen and an adjuvant (for examples of relevant antigen, see PCT/GB2006/000987, which is incorporated by reference in its entirety). It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. In some embodiments, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

### (iii) Monoclonal Antibodies

Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as herein described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In some embodiments, the myeloma cells are those that fuse efficiently, support stable high-level production of antibody by the selected antibody-producing cells, and are sensitive to a medium such as HAT medium. Among these, in some embodiments, the myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. In some embodiments, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). In some embodiments, the hybridoma cells serve as a source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In some embodiments, the monoclonal anti-HCV antibody is AP33 antibody. See PCT/GB2006/000987, which is incorporated by reference in its entirety.

In some embodiments, the monoclonal anti-HCV antibody preferably comprises one, two, three, four, five or six of the following CDR sequences:

CDR L1 sequence RASESVDGYGNSFLH, (SEQ ID NO:41)

CDR L2 sequence LASNLNS, (SEQ ID NO:42)

CDR L3 sequence QQNNVDPWT, (SEQ ID NO:43)

CDR H1 sequence of GDSITSGYWN, (SEQ ID NO:44)

CDR H2 sequence of YISYSGSTY (SEQ ID NO:45), and

CDR H3 sequence of ITTTTYAMDY. (SEQ ID NO:46)

In some embodiments, the monoclonal anti-HCV antibody preferably comprises one, two, three, four, five or six of the following CDR sequences:

CDR L1 sequence RASESVDGYGNSFLH, (SEQ ID NO:41)

CDR L2 sequence LASNLNS, (SEQ ID NO:42)

CDR L3 sequence QQNNVDPWT, (SEQ ID NO:43)

CDR H1 sequence of SGYWN, (SEQ ID NO:47)

CDR H2 sequence of YISYSGSTYYNLSLRS (SEQ ID NO:48), and

CDR H3 sequence of ITTTTYAMDY. (SEQ ID NO:46)

In some embodiments of any of the monoclonal antibodies, the monoclonal antibody described herein bind to HCV. In some embodiments, the monoclonal antibody is capable of binding to HCV E2 protein, soluble HCV E2 protein, or a heterodimer of HCV E1 protein and HCV E2 protein. In some embodiments, the monoclonal antibody binds HCV E2 protein. In some embodiments, the HCV E2 protein is from one or more of the HCV genotypes selected from the group consisting of genotype 1 (e.g., genotype 1a and genotype 1b), genotype 2 (e.g., genotype 2a, genotype 2b, genotype 2c), genotype 3 (e.g., genotype 3a), genotype 4, genotype 5, and genotype 6. In some embodiments, the monoclonal antibody inhibits the interaction of HCV E2 protein with CD81. In some embodiments, the monoclonal antibody prevents and/or inhibits HCV entry into the cell. In some embodiments, the cell is a liver cell, e.g., hepatocyte. In some embodiments, the monoclonal antibody is an antibody fragment.

In some embodiments, the monoclonal antibody binds to soluble HCV E2 protein with a binding affinity of between 1-100 nM. In some embodiments, the binding affinity is between about any of 1-10 nM, 10-50 nM, or 50-100 nM. In some embodiments, the binding affinity is about 5 nM or about 50 nM. In some embodiments, the humanized antibody binds to HCV E1/HCV E2 heterodimer with a binding affinity of between 1-100 nM. In some embodiments, the binding affinity is between about any of 1-10 nM, 10-50 nM, or 50-100 nM. In some embodiments, the binding affinity is about 5 nM or about 50 nM. In some embodiments, the binding affinity of the monoclonal antibody can, for example, be determined by the Scatchard analysis described in Munson et al., Anal. Biochem., 107:220 (1980).

In some embodiments, the monoclonal antibody described herein inhibits HCV infection. In some embodiments, the monoclonal antibody described herein inhibits HCV pseudoparticle (HCVpp) infection. In some embodiments, the monoclonal antibody described herein inhibits recombinant cell culture-derived HCV (HCVcc) infection.

In some embodiments, the monoclonal antibody exhibits one or more of the above characteristics.

### (iv) HUMANIZED ANTIBODIES

Methods for humanizing non-human antibodies have been described in the art. In some embodiments, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, in some embodiments of the methods, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

In some embodiments, the humanized anti-HCV antibody comprises one, two, three, four, five or six of the following CDR sequences:

CDR L1 sequence RASESVDGYGNSFLH, (SEQ ID NO:41)

CDR L2 sequence LASNLNS, (SEQ ID NO:42)

CDR L3 sequence QQNNVDPWT, (SEQ ID NO:43)

CDR H1 sequence of GDSITSGYWN, (SEQ ID NO:44)

CDR H2 sequence of YISYSGSTY (SEQ ID NO:45), and

CDR H3 sequence of ITTTTYAMDY. (SEQ ID NO:46)

In some embodiments, the humanized anti-HCV antibody comprises one, two, three, four, five or six of the following CDR sequences:

CDR L1 sequence RASESVDGYGNSFLH, (SEQ ID NO:41)

CDR L2 sequence LASNLNS, (SEQ ID NO:42)

CDR L3 sequence QQNNVDPWT, (SEQ ID NO:43)

CDR H1 sequence of SGYWN, (SEQ ID NO:47)

CDR H2 sequence of YISYSGSTYYNLSLRS (SEQ ID NO:48), and

CDR H3 sequence of ITTTTYAMDY. (SEQ ID NO:46)

The CDR sequences above are generally present within human variable light and variable heavy framework sequences, such as substantially the human consensus FR residues of human light chain kappa subgroup I (VL6I), and substantially the human consensus FR residues of human heavy chain subgroup III (VHIII). See also WO 2004/056312 (Lowman et al.).

In some embodiments, the variable heavy region may be joined to a human IgG chain constant region, wherein the region may be, for example, IgG1 or IgG3, including native sequence and variant constant regions.

In one embodiment, there is provided a variable light chain domain of a humanized AP33 antibody comprising the amino acid sequence set forth in any of in any of SEQ ID NO:6, SEQ ID NO:7, SEQ ID NO:19, or SEQ ID NO:20.

In another embodiment, there is provided a variable heavy chain domain of a humanized AP33 antibody comprising amino acid mutations at positions 30, 48, 67, 71 78 and 94 of SEQ ID No. 3.

The amino acid mutation may be obtained by substitution of one or more amino acid residue(s). In certain circumstances, a deletion or insertion may be tolerated. Mutation can be carried out using standard techniques such as for example site directed mutagenesis.

Suitably, the amino acid mutations are substitutions.

In another embodiment, the variable heavy chain domain according comprises the amino acid sequence as set forth in any of SEQ ID NO:10, SEQ ID NO:11, SEQ ID NO:12, SEQ ID NO:13, SEQ ID NO:14, SEQ ID NO:15, SEQ ID NO:16, SEQ ID NO:17, or SEQ ID NO:18.

There is also provided a humanized antibody or humanized antibody fragment comprising the variable light chain domain.

There is also provided a humanized antibody or humanized antibody fragment comprising the variable heavy chain domain.

There is also provided a humanized antibody or humanized antibody fragment comprising a light chain and a heavy chain, wherein the variable region of the light chain and the variable region of the heavy chain are as defined herein.

In some embodiments, the humanized antibody comprises a variable heavy chain domain selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18 and a variable light chain domain selected from the group consisting of SEQ ID NO:6, SEQ ID NO:7, SEQ ID NO:19, and SEQ ID NO:20.

In some embodiments, the variable heavy chain domain is selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18 and the variable light chain domain is SEQ ID NO:6. In some embodiments, the variable heavy chain domain is selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18 and the variable light chain domain is SEQ ID NO:7. In some embodiments, the variable heavy chain domain is selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18 and the variable light chain domain is SEQ ID NO:19. In some embodiments, the variable heavy chain domain is SEQ ID NO: 13 and the variable light chain domain is SEQ ID NO:19. In some embodiments, the variable heavy chain domain is selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18 and the variable light chain domain is SEQ ID NO:20.

In some embodiments of any of the humanized antibodies, the humanized antibody described herein bind to HCV. In some embodiments, the humanized antibody is capable of binding to HCV E2 protein, soluble HCV E2 protein, or a heterodimer of HCV E1 protein and HCV E2 protein. In some embodiments, the humanized antibody binds HCV E2 protein. In some embodiments, the HCV E2 protein is from one or more of the HCV genotypes selected from the group consisting of genotype 1 (e.g., genotype 1a and genotype 1b), genotype 2 (e.g., genotype 2a, genotype 2b, genotype 2c), genotype 3 (e.g., genotype 3a), genotype 4, genotype 5, and genotype 6. In some embodiments, the humanized antibody inhibits the interaction of HCV E2 protein with CD81. In some embodiments, the humanized antibody prevents and/or inhibits HCV entry into the cell. In some embodiments, the cell is a liver cell, e.g., hepatocyte. In some embodiments, the humanized antibody is an antibody fragment.

In some embodiments, the humanized antibody binds to soluble HCV E2 protein with a binding affinity of between 1-100 nM. In some embodiments, the binding affinity is between about any of 1-10 nM, 10-50 nM, or 50-100 nM. In some embodiments, the binding affinity is about 5 nM or about 50 nM. In some embodiments, the humanized antibody binds to HCV E1/HCV E2 heterodimer with a binding affinity of between 1-100 nM. In some embodiments, the binding affinity is between about any of 1-10 nM, 10-50 nM, or 50-100 nM. In some embodiments, the binding affinity is about 5 nM or about 50 nM. In some embodiments, the binding affinity of the antibody can, for example, be determined by the Scatchard analysis described in Munson et al., Anal. Biochem., 107:220 (1980).

In some embodiments, the humanized antibody described herein inhibits HCV infection. In some embodiments, the humanized antibody described herein inhibits HCV pseudoparticle (HCVpp) infection. Suitably, the humanized antibody as described herein is capable of inhibiting HCV pseudoparticle infection wherein the IC50 of infectious titers in the presence of said humanized antibody as judged by the HCVpp neutralization assay is: at least about 0.032 for genotype 1 (1a H77 20); at least about 1.6 for genotype 1 (1A20.8); at least about 0.9 for genotype 1 (1B5.23); at least about 3 for genotype 2 (2B1.1); at least about 0.41 for genotype 3a (F4/2-35); at least about 0.41 for genotype 4 (4.21.16); at least about 0.41 for genotype 6 (6.5.8); and at least 0.053 for genotype 5 (5.15.11). In some embodiments, the humanized antibody or fragment thereof as described herein is capable of inhibiting HCV pseudoparticle infection wherein the IC50 of infectious titers in the presence of said humanized antibody as judged by the HCVpp neutralization assay is any of less than about 0.41, less than about 0.137, or about 0.32 for genotype 1 (1a H77 20), about 1.6 for genotype 1 (1A20.8), about 0.9 for genotype 1 (1B5.23), about 3 for genotype 2 (2B1.1), about 0.64 for genotype 2 (2a JFH1), about 0.51 for genotype 2 (2A2.4), less than about 0.41 for genotype 3 (3a F4/2-35), less than about 0.41 for genotype 4 (4.21.16), about 0.053 for genotype 5 (5.15.11), or less than about 0.41 for genotype 6 (6.5.8).

In some embodiments, the humanized antibody as described herein is capable of inhibiting HCVpp infection wherein the EC₅₀ of infectious titers in the presence of said humanized antibody as judged by the HCVpp neutralization assay is: at least about 0.511 for genotype 1b or at least about 0.793 for genotype 2a.

Suitably, the IC90 of infectious titers in the presence of said humanized antibody as judged by the HCVpp neutralization assay is: at least about 0.6 for genotype 1 (1a H77 20); at least about 15 for genotype 1 (1A20.8); at least about 8.3 for genotype 1 (1B5.23); at least about 15 for genotype 2 (2B1.1); at least about 2.15 for genotype 3a (D4/2-35); at least about 0.92 for genotype 4 (4.21.16); at least about 1.8 for genotype 6 (6.5.8); and at least 0.82 for genotype 5 (5.15.11). In some embodiments, the humanized antibody or fragment thereof as described herein is capable of inhibiting HCV pseudoparticle infection wherein the IC90 of infectious titers in the presence of said humanized antibody as judged by the HCVpp neutralization assay is any of less than about 0.41, about 2.4, or about 0.6 for genotype 1 (1a H77 20), about 15 for genotype 1 (1A20.8), about 8.3 for genotype 1 (1B5.23), greater than about 15 for genotype 2 (2B1.1), about 7 for genotype 2 (2a JFH1), about 0.51 for genotype 2 (2A2.4), less than about 0.41 for genotype 3 (3a F4/2-35), less than about 6 for genotype 4 (4.21.16), about 0.82 for genotype 5 (5.15.11), or less than about 1.8 for genotype 6 (6.5.8).

In some embodiments, the humanized antibody described herein inhibits recombinant cell culture-derived HCV (HCVcc) infection. In some embodiments, the humanized antibody as described herein is capable of inhibiting HCVcc infection wherein the EC₅₀ of infectious titers in the presence of said humanized antibody as judged by the HCVpp neutralization assay is: at least about 0.72 for genotype 1b or at least about 1.7 for genotype 2a.

In some embodiments, the humanized antibody or fragment thereof exhibits one or more of the above characteristics.

### (v) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also, U.S. Pat. Nos. 5,565,332 and 5,573,905.

Human antibodies may also be generated by in vitro activated B cells (see U.S. Pat. Nos. 5,567,610 and 5,229,275).

### (vi) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

In some embodiments, fragments of the antibodies described herein are provided. In some embodiments, the antibody fragments are antigen binding fragments. In some embodiments, the antigen binding fragments of the antibody bind to HCV. In some embodiments, the antigen binding fragments of the antibody are capable of binding to HCV E2 protein, soluble HCV E2 protein, or a heterodimer of HCV E1 protein and HCV E2 protein. In some embodiments, the HCV E2 protein is from one or more of the HCV genotypes selected from the group consisting of genotype 1 (e.g., genotype 1a and genotype 1b), genotype 2 (e.g., genotype 2a, genotype 2b, genotype 2c), genotype 3 (e.g., genotype 3a), genotype 4, genotype 5, and genotype 6.

Typically, these fragments exhibit specific binding to antigen with an affinity of at least 10⁷, and more typically 10⁸ or 10⁹. In some embodiments, the humanized antibody fragment binds to soluble HCV E2 protein with a binding affinity of between 1-100 nM. In some embodiments, the binding affinity is between about any of 1-10 nM, 10-50 nM, or 50-100 nM. In some embodiments, the binding affinity is about 5 nM or about 50 nM. In some embodiments, the antibody binds to HCV E1/HCV E2 heterodimer with a binding affinity of between 1-100 nM. In some embodiments, the binding affinity is between about any of 1-10 nM, 10-50 nM, or 50-100 nM. In some embodiments, the binding affinity is about 5 nM or about 50 nM. In some embodiments, the binding affinity of the antibody can, for example, be determined by the Scatchard analysis described in Munson et al., Anal. Biochem., 107:220 (1980).

In some embodiments, these fragments exhibit (substantially) the same HCV neutralizing activity as the AP33 monoclonal antibody or the humanized antibody described herein.

In some embodiments, the humanized antibody fragments are functional fragments. “Functional fragments” of the humanized antibodies described herein such as functional fragments of the humanized AP33 antibody are those fragments that retain binding to HCV with substantially the same affinity as the intact full length molecule from which they are derived and show biological activity as measured by in vitro or in vivo assays such as those described herein. In some embodiments, the functional fragment neutralizes and/or inhibits HCV as shown by HCVpp and/or HCVcc neutralization assays. In some embodiments, the humanized antibody fragment prevents and/or inhibits the interaction of HCV E2 protein with CD81. In some embodiments, the humanized antibody fragment prevents and/or inhibits HCV entry into the cell. In some embodiments, the cell is a liver cell, e.g., hepatocyte.

### (vii) Bispecific Antibodies

Bispecific antibodies are antibodies that have binding specificities for at least two different epitopes. Exemplary bispecific antibodies may bind to two different epitopes of the B cell surface marker. Other such antibodies may bind the B cell surface marker and further bind a second different B-cell surface marker. Alternatively, an anti-B cell surface marker binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the B cell. Bispecific antibodies may also be used to localize cytotoxic agents to the B cell. These antibodies possess a B cell surface marker-binding arm and an arm that binds the cytotoxic agent (e.g. saporin, anti-interferon-a, vinca alkaloid, ricin A chain, methotrexate or radioactive isotope hapten). Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. In some embodiments, the fusion is with an immunoglobulin heavy chain constant domain, comprising at least part of the hinge, CH2, and CH3 regions. In some embodiments, the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some embodiments of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. In some embodiments, the interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

### (viii) Multivalent Antibodies

A multivalent antibody may be internalized (and/or catabolized) faster than a bivalent antibody by a cell expressing an antigen to which the antibodies bind. The antibodies provided herein can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### (ix) Other Amino Acid Sequence Modifications

Amino acid sequence modification(s) of the HCV binding antibodies described herein are contemplated. For example, it may be desirable to improve the binding affinity and/or other biological properties of the antibody. Amino acid sequence variants of the anti-HCV antibody such as humanized AP33 antibodies are prepared by introducing appropriate nucleotide changes into the anti-HCV antibody nucleic acid, or by peptide synthesis. Such modifications include, for example, deletions from, and/or insertions into and/or substitutions of, residues within the amino acid sequences of the anti-HCV antibody. Any combination of deletion, insertion, and substitution is made to arrive at the final construct, provided that the final construct possesses the desired characteristics. The amino acid changes also may alter post-translational processes of the anti-HCV antibody, such as changing the number or position of glycosylation sites.

A useful method for identification of certain residues or regions of the anti-HCV antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with HCV antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-HCV antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-HCV antibody with an N-terminal methionyl residue or the antibody fused to a cytotoxic polypeptide. Other insertional variants of the anti-HCV antibody molecule include the fusion to the N- or C-terminus of the anti-HCV antibody to an enzyme (e.g. for ADEPT) or a polypeptide which increases the serum half-life of the antibody.

Another type of variant is an amino acid substitution variant. These variants have at least one amino acid residue in the anti-HCV antibody molecule replaced by a different residue. The sites of greatest interest for substitutional mutagenesis include the hypervariable regions, but FR alterations are also contemplated. Conservative substitutions are shown in the Table below under the heading of “preferred substitutions”. If such substitutions result in a change in biological activity, then more substantial changes, denominated “exemplary substitutions” in the Table, or as further described below in reference to amino acid classes, may be introduced and the products screened.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (O)

(3) acidic: Asp (D), Glu (E)

(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: H is, Lys, Arg;

(5) residues that influence chain orientation: Gly, Pro;

(6) aromatic: Trp, Tyr, Phe.

Non-conservative substitutions will entail exchanging a member of one of these classes for another class.

Any cysteine residue not involved in maintaining the proper conformation of the anti-HCV antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g., a humanized antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and HCV. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. The humanized antibodies may comprise non-amino acid moieties. For example, the humanized antibodies may be glycosylated. Such glycosylation may occur naturally during expression of the humanized antibodies in the host cell or host organism, or may be a deliberate modification arising from human intervention. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-HCV antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-HCV antibody.

It may be desirable to modify the antibody provided herein with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement mediated lysis and ADCC capabilities. See Stevenson et al., Anti-Cancer Drug Design 3:219-230 (1989).

For increasing serum half the serum half life of the antibody, amino acid alterations can be made in the antibody as described in US 2006/0067930, which is hereby incorporated by reference in its entirety.

### (x) Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

Additionally or alternatively the humanized antibodies may be subjected to other chemical modification. One such desirable modification is addition of one or more polyethylene glycol (PEG) moieties. Pegylation has been shown to increase significantly the half-life of various antibody fragments in vivo (Chapman 2002 Adv. Drug Delivery Rev. 54, 531-545). However, random Pegylation of antibody fragments can have highly detrimental effects on the binding affinity of the fragment for the antigen. In order to avoid this it is desirable that Pegylation is restricted to specific, targeted residues of the humanized antibodies (see Knight et al, 2004 Platelets 15, 409-418 and Chapman, supra).

### (xi) Screening for Antibodies with Desired Properties

Antibodies with certain biological characteristics may be selected as described in the Experimental Examples.

To screen for antibodies which bind to an epitope on the HCV E2 protein bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as an anti-HCV E2 antibody provided herein. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of HCV E2 protein can be used in competition assays with the test antibodies or with a test antibody and an antibody with a characterized or known epitope.

In some embodiments, antibodies can also be screen for their ability to neutralize an HCV infection. In some embodiments, neutralization of an HCV infection is based on a HCV pseudotyped particles (HCVpp) neutralization assay as described herein. HCVpp consist of unmodified HCV envelop glycoproteins assembled onto retroviral or lentiviral core particles. HCVpp infect hepatoma cell lines and hepatocytes in an HCV envelop protein-dependent matter. The presence of a marker gene packaged within the HCVpp allows fast and reliable determination of antibody-mediated neutralization. In some embodiments, neutralization of an HCV infection is based on a recombinant cell culture-derived HCV (HCVcc) neutralization assay infecting human hepatoma cell lines as described herein.

### III. Polynucleotides

Provided herein are also polynucleotide(s) encoding the antibodies or antibody fragments described herein.

“Polynucleotide,” or “nucleic acid,” as used interchangeably herein, refer to polymers of nucleotides of any length, and include DNA and RNA.

For example, the polynucleotide may encode an entire immunoglobulin molecule chain, such as a light chain or a heavy chain. A complete heavy chain includes not only a heavy chain variable region (V_{H}) but also a heavy chain constant region (C_{H}), which typically will comprise three constant domains: C_{H}1, C_{H}2 and C_{H}3; and a “hinge” region. In some situations, the presence of a constant region is desirable. For example, where the antibody is desired to kill an HCV-infected cell, the presence of a complete constant region is desirable to activate complement. However, in other situations the presence of a complete constant region may be undesirable.

The polynucleotide may encode a variable light chain and/or a variable heavy chain.

Other polypeptides which may be encoded by the polynucleotide include antigen-binding antibody fragments such as single domain antibodies (“dAbs”), Fv, scFv, Fab′ and F(ab′)₂ and “minibodies”. Minibodies are (typically) bivalent antibody fragments from which the C_{H}1 and C_{K} or C_{L} domain has been excised. As minibodies are smaller than conventional antibodies they should achieve better tissue penetration in clinical/diagnostic use, but being bivalent they should retain higher binding affinity than monovalent antibody fragments, such as dAbs. Accordingly, unless the context dictates otherwise, the term “antibody” as used herein encompasses not only whole antibody molecules but also antigen-binding antibody fragments of the type discussed above.

Whilst the encoded polypeptide will typically have CDR sequences identical or substantially identical to those of AP33, the framework regions will differ from those of AP33, being of human origin. The polynucleotide will thus preferably encode a polypeptide having a heavy and/or light chain variable region as described herein relative to the heavy and/or light chain (as appropriate) of AP33. If the encoded polypeptide comprises a partial or complete heavy and/or light chain constant region, this too is advantageously of human origin.

Preferably at least one of the framework regions of the encoded polypeptide, and most preferably each of the framework regions, will comprise amino acid substitutions relative to the human acceptor so as to become more similar to those of AP33, so as to increase the binding activity of the humanized antibody.

Preferably each framework region present in the encoded polypeptide will comprise at least one amino acid substitution relative to the corresponding human acceptor framework. Thus, for example, the framework regions may comprise, in total, three, four, five, six, seven, eight, nine, ten, eleven, twelve, thirteen, fourteen or fifteen amino acid substitutions relative to the acceptor framework regions. Advantageously, the mutations are backmutations to match the residues present at the equivalent positions in the murine AP33 framework. Preferably, six backmutations are made in the heavy chain and one in the light chain.

Suitably, the polynucleotide and/or the polypeptide described herein may be isolated and/or purified. In some embodiments, the polynucleotide and/or polypeptide are an isolated polynucleotide and/or polypeptide. The term isolated is intended to indicate that the molecule is removed or separated from its normal or natural environment or has been produced in such a way that it is not present in its normal or natural environment. In some embodiments, the polynucleotide and/or polypeptide are a purified polynucleotide and/or polypeptide. The term purified is intended to indicate that at least some contaminating molecules or substances have been removed.

Suitably, the polynucleotide and/or polypeptide are substantially purified, such that the relevant polynucleotide and/or polypeptide constitutes the dominant (i.e., most abundant) polynucleotide or polypeptide present in a composition.

Recombinant nucleic acids comprising an insert coding for a heavy chain variable domain and/or for a light chain variable domain may be used in the methods as described herein. By definition such nucleic acids comprise coding single stranded nucleic acids, double stranded nucleic acids consisting of said coding nucleic acids and of complementary nucleic acids thereto, or these complementary (single stranded) nucleic acids themselves.

Modification(s) may also be made outside the heavy chain variable domain and/or of the light chain variable domain of the AP33 antibody. Such a mutant nucleic acid may be a silent mutant wherein one or more nucleotides are replaced by other nucleotides with the new codons coding for the same amino acid(s). Such a mutant sequence may be a degenerate sequence. Degenerate sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly yeast, bacterial or mammalian cells, to obtain an optimal expression of the heavy chain variable domain and/or the light chain variable domain.

Provided herein is also the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

In some embodiments, homologous amino acid sequence and/or nucleotide sequence should provide and/or encode a polypeptide which retains the functional activity and/or enhances the activity of the antibody. In some embodiments, homologous sequence is taken to include an amino acid sequence which may be at least 75, 85, or 90% identical, preferably at least 95 or 98% identical to the subject sequence. Typically, the homologues will comprise the same active sites etc. as the subject amino acid sequence. Although homology can also be considered in terms of similarity (i.e., amino acid residues having similar chemical properties/functions). In some embodiments, it is preferred to express homology in terms of sequence identity.

In the present context, a homologous sequence is taken to include a nucleotide sequence which may be at least 75, 85, or 90% identical, preferably at least 95 or 98% identical to a nucleotide sequence encoding a polypeptide described herein (the subject sequence). Typically, the homologues will comprise the same sequences that code for the active sites etc. as the subject sequence. Although homology can also be considered in terms of similarity (i.e., amino acid residues having similar chemical properties/functions). In some embodiments, it is preferred to express homology in terms of sequence identity.

In a further aspect, there is provided a polynucleotide sequence that is capable of hybridizing (e.g. specifically hybridizing) to the polynucleotide sequence(s) described herein.

The term “hybridization” as used herein shall include “the process by which a strand of polynucleotide joins with a complementary strand through base pairing”. Hybridization conditions are based on the melting temperature (Tm) of the nucleic acid binding complex, as taught in Berger and Kimmel (1987, Guide to Molecular Cloning Techniques, Methods in Enzymology, 152, Academic Press, San Diego Calif.), and confer a defined “stringency” as explained below.

Maximum stringency typically occurs at about 5° C. below Tm; high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related sequences.

In some embodiments, the polynucleotide sequence(s) that is capable of hybridizing to the nucleotide sequence(s) described herein is a sequence that is capable of hybridizing under stringent conditions (e.g., 50° C. and 0.2×SSC {1×SSC=0.15 M NaCl, 0.015 M Na₃citrate pH 7.0}) to the polynucleotide sequences described herein. In some embodiments, the polynucleotide sequence(s) that is capable of hybridizing under high stringent conditions (e.g., 65° C. and 0.1×SSC {1×SSC=0.15 M NaCl, 0.015 M Na₃citrate pH 7.0}) to the polynucleotide sequences presented herein.

Provided herein are also polynucleotide sequences that are complementary to sequences that can hybridize to the polynucleotide sequences of the present invention (including complementary sequences of those presented herein).

Further, provided herein are nucleotide sequences that are capable of hybridizing to the nucleotide sequences presented herein under conditions of intermediate to maximal stringency.

### IV. Expression of Recombinant Antibodies

Also provided are isolated polynucleotides encoding the anti-HCV antibodies described herein such as the humanized AP33 antibodies, vectors and host cells comprising the polynucleotide, and recombinant techniques for the production of the antibody. The antibodies described herein may be produced by recombinant expression.

Polynucleotide encoding light and heavy chain variable regions as described herein are optionally linked to constant regions, and inserted into an expression vector(s). The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences.

Suitably, the expression control sequences are eukaryotic promoter systems in vectors capable of transforming or transfecting eukaryotic host cells (e.g., COS cells—such as COS 7 cells—or CHO cells). Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA.

Selection Gene Component—Commonly, expression vectors contain selection markers (e.g., ampicillin-resistance, hygromycin-resistance, tetracycline resistance, kanamycin resistance or neomycin resistance) to permit detection of those cells transformed with the desired DNA sequences (see, e.g., Itakura et al., U.S. Pat. No. 4,704,362). In some embodiments, selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the nucleic acid encoding anti-HCV antibodies described herein such as the humanized AP33 antibodies, such as DHFR, thymidine kinase, metallothionein-I and -III, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody described herein, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

Signal Sequence Component—The anti-HCV antibodies described herein such as the humanized AP33 antibodies may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. A signal sequence can be substituted with a prokaryotic signal sequence selected, for example, from the group of the alkaline phosphatase, penicillinase, 1 pp, or heat-stable enterotoxin II leaders. For yeast secretion the native signal sequence may be substituted by, e.g., the yeast invertase leader, a factor leader (including Saccharomyces and Kluyveromyces a-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the anti-HCV antibodies described herein such as the humanized AP33 antibodies.

Origin of Replication—Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Promoter Component—Expression and cloning vectors usually contain a promoter that is recognized by the host organism and is operably linked to the nucleic acid encoding an antibody described herein such as a humanized AP33 antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the anti-HCV antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

The transcription of an anti-HCV antibody described herein such as the humanized AP33 antibody from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human .beta.-interferon cDNA in mouse cells under the control of a thymidine kinase promoter from herpes simplex virus. Alternatively, the Rous Sarcoma Virus long terminal repeat can be used as the promoter.

Enhancer Element Component—Transcription of a DNA encoding the anti-HCV antibody described herein such as the humanized AP33 antibody by higher eukaryotes is often increased by inserting an enhancer sequence into the vector. Many enhancer sequences are now known from mammalian genes (globin, elastase, albumin, .alpha.-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the HCV binding antibody-encoding sequence, but is preferably located at a site 5′ from the promoter.

Transcription Termination Component—Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. One useful transcription termination component is the bovine growth hormone polyadenylation region. See WO94/11026 and the expression vector disclosed therein.

The vectors containing the polynucleotide sequences (e.g., the variable heavy and/or variable light chain encoding sequences and optional expression control sequences) can be transferred into a host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

When heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity is most preferred, for pharmaceutical uses.

### (i) Constructs

The invention further provides a nucleic acid construct comprising a polynucleotide as described herein.

Typically the construct will be an expression vector allowing expression, in a suitable host, of the polypeptide(s) encoded by the polynucleotide. The construct may comprise, for example, one or more of the following: a promoter active in the host; one or more regulatory sequences, such as enhancers; an origin of replication; and a marker, preferably a selectable marker. The host may be a eukaryotic or prokaryotic host, although eukaryotic (and especially mammalian) hosts may be preferred. The selection of suitable promoters will obviously depend to some extent on the host cell used, but may include promoters from human viruses such as HSV, SV40, RSV and the like. Numerous promoters are known to those skilled in the art.

The construct may comprise a polynucleotide which encodes a polypeptide comprising three light chain hypervariable loops or three heavy chain hypervariable loops. Alternatively the polynucleotide may encode a polypeptide comprising three heavy chain hypervariable loops and three light chain hypervariable loops joined by a suitably flexible linker of appropriate length. Another possibility is that a single construct may comprise a polynucleotide encoding two separate polypeptides—one comprising the light chain loops and one comprising the heavy chain loops. The separate polypeptides may be independently expressed or may form part of a single common operon.

The construct may comprise one or more regulatory features, such as an enhancer, an origin of replication, and one or more markers (selectable or otherwise). The construct may take the form of a plasmid, a yeast artificial chromosome, a yeast mini-chromosome, or be integrated into all or part of the genome of a virus, especially an attenuated virus or similar which is non-pathogenic for humans.

The construct may be conveniently formulated for safe administration to a mammalian, preferably human, subject. Typically, they will be provided in a plurality of aliquots, each aliquot containing sufficient construct for effective immunization of at least one normal adult human subject.

The construct may be provided in liquid or solid form, preferably as a freeze-dried powder which, typically, is rehydrated with a sterile aqueous liquid prior to use.

The construct may be formulated with an adjuvant or other component which has the effect of increasing the immune response of the subject (e.g., as measured by specific antibody titer) in response to administration of the construct.

### (ii) Vectors

The term “vector” includes expression vectors and transformation vectors and shuttle vectors.

The term “expression vector” means a construct capable of in vivo or in vitro expression.

The term “transformation vector” means a construct capable of being transferred from one entity to another entity—which may be of the species or may be of a different species. If the construct is capable of being transferred from one species to another—such as from an Escherichia coli plasmid to a bacterium, such as of the genus Bacillus, then the transformation vector is sometimes called a “shuttle vector”. It may even be a construct capable of being transferred from an E. coli plasmid to an Agrobacterium to a plant.

Vectors may be transformed into a suitable host cell as described below to provide for expression of a polypeptide encompassed in the present invention. Thus, in a further aspect the invention provides a process for preparing polypeptides for use in the present invention which comprises cultivating a host cell transformed or transfected with an expression vector as described above under conditions to provide for expression by the vector of a coding sequence encoding the polypeptides, and recovering the expressed polypeptides.

The vectors may be for example, plasmid, virus or phage vectors provided with an origin of replication, optionally a promoter for the expression of the said polynucleotide and optionally a regulator of the promoter.

Vectors may contain one or more selectable marker genes which are well known in the art.

(iii) Host Cells

The invention further provides a host cell—such as a host cell in vitro—comprising the polynucleotide or construct described herein. The host cell may be a bacterium, a yeast or other fungal cell, insect cell, a plant cell, or a mammalian cell, for example.

The invention also provides a transgenic multicellular host organism which has been genetically manipulated so as to produce a polypeptide in accordance with the invention. The organism may be, for example, a transgenic mammalian organism (e.g., a transgenic goat or mouse line).

E. coli is one prokaryotic host that may be of use. Other microbial hosts include bacilli, such as Bacillus subtilis, and other enterobacteriaceae, such as Salmonella, Serratia, and various Pseudomonas species. In these prokaryotic hosts, one can make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, may be used for expression. Saccharomyces is a preferred yeast host, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian tissue cell culture may also be used to express and produce the humanized antibodies as described herein and in some instances are preferred (See Winnacker, From Genes to Clones, VCH Publishers, N.Y., N.Y. (1987). For some embodiments, eukaryotic cells (e.g., COS7 cells) may be preferred, because a number of suitable host cell lines capable of secreting heterologous proteins (e.g., intact immunoglobulins) have been developed in the art, and include CHO cell lines, various Cos cell lines, HeLa cells, preferably, myeloma cell lines, or transformed B-cells or hybridomas.

In some embodiments, the host cell is a vertebrate host cell. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR(CHO, Urlaub et al., Proc. Natl. Acad. Sci. USA 77:4216 (1980)) or CHO-DP-12 line; mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Alternatively, antibody-coding sequences can be incorporated into transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

Alternatively, the antibodies described herein can be produced in transgenic plants (e.g., tobacco, maize, soybean and alfalfa). Improved ‘plantibody’ vectors (Hendy et al. (1999) J. Immunol. Methods 231:137-146) and purification strategies coupled with an increase in transformable crop species render such methods a practical and efficient means of producing recombinant immunoglobulins not only for human and animal therapy, but for industrial applications as well (e.g., catalytic antibodies). Moreover, plant produced antibodies have been shown to be safe and effective and avoid the use of animal-derived materials. Further, the differences in glycosylation patterns of plant and mammalian cell-produced antibodies have little or no effect on antigen binding or specificity. In addition, no evidence of toxicity or HAMA has been observed in patients receiving topical oral application of a plant-derived secretory dimeric IgA antibody (see Larrick et al. (1998) Res. Immunol. 149:603-608).

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

Suitable host cells for the expression of glycosylated anti-HCV antibodies such as a humanized AP33 antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

### (iv) Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C.sub.H3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### V. Antibody Conjugates

The antibody may be conjugated to a cytotoxic agent such as a toxin or a radioactive isotope. In certain embodiments, the toxin is calicheamicin, a maytansinoid, a dolastatin, auristatin E and analogs or derivatives thereof, are preferable.

Preferred drugs/toxins include DNA damaging agents, inhibitors of microtubule polymerization or depolymerization and antimetabolites. Preferred classes of cytotoxic agents include, for example, the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins and differentiation inducers. Particularly useful members of those classes include, for example, methotrexate, methopterin, dichloromethotrexate, 5-fluorouracil, 6-mercaptopurine, cytosine arabinoside, melphalan, leurosine, leurosideine, actinomycin, daunorubicin, doxorubicin, N-(5,5-diacetoxypentyl)doxorubicin, morpholino-doxorubicin, 1-(2-choroehthyl)-1,2-dimethanesulfonyl hydrazide, N⁸-acetyl spermidine, aminopterin methopterin, esperamicin, mitomycin C, mitomycin A, actinomycin, bleomycin, caminomycin, aminopterin, tallysomycin, podophyllotoxin and podophyllotoxin derivatives such as etoposide or etoposide phosphate, vinblastine, vincristine, vindesine, taxol, taxotere, retinoic acid, butyric acid, camptothecin, calicheamicin, bryostatins, cephalostatins, ansamitocin, actosin, maytansinoids such as DM-1, maytansine, maytansinol, N-desmethyl-4,5-desepoxymaytansinol, C-19-dechloromaytansinol, C-20-hydroxymaytansinol, C-20-demethoxymaytansinol, C-9-SH maytansinol, C-14-alkoxymethylmaytansinol, C-14-hydroxy or acetyloxymethlmaytansinol, C-15-hydroxy/acetyloxymaytansinol, C-15-methoxymaytansinol, C-18-N-demethylmaytansinol and 4,5-deoxymaytansinol, auristatins such as auristatin E, M, PHE and PE; dolostatins such as dolostatin A, dolostatin B, dolostatin C, dolostatin D, dolostatin E (20-epi and 11-epi), dolostatin G, dolostatin H, dolostatin I, dolostatin 1, dolostatin 2, dolostatin 3, dolostatin 4, dolostatin 5, dolostatin 6, dolostatin 7, dolostatin 8, dolostatin 9, dolostatin 10, deo-dolostatin 10, dolostatin 11, dolostatin 12, dolostatin 13, dolostatin 14, dolostatin 15, dolostatin 16, dolostatin 17, and dolostatin 18; cephalostatins such as cephalostatin 1, cephalostatin 2, cephalostatin 3, cephalostatin 4, cephalostatin 5, cephalostatin 6, cephalostatin 7, 25′-epi-cephalostatin 7,20-epi-cephalostatin 7, cephalostatin 8, cephalostatin 9, cephalostatin 10, cephalostatin 11, cephalostatin 12, cephalostatin 13, cephalostatin 14, cephalostatin 15, cephalostatin 16, cephalostatin 17, cephalostatin 18, and cephalostatin 19.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene.

There are many linking groups known in the art for making antibody-maytansinoid conjugates, including, for example, those disclosed in U.S. Pat. No. 5,208,020 or EP Patent 0 425 235 B1, and Chari et al. Cancer Research 52: 127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio)propionate (SPDP) (Carlsson et al., Biochem. J. 173:723-737 119781) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Another immunoconjugate of interest comprises an anti-HCV antibody such as a humanized APP-33 antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, a₂^{I}, a₃^{I}, N-acetyl-γ₁^{I}, PSAG and θ¹₁ (Hinman et al. Cancer Research 53: 3336-3342 (1993), Lode et al. Cancer Research 58: 2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Radioactive Isotopes

For selective destruction of an HCV infected cell, the antibody may comprise a highly radioactive atom. A variety of radioactive isotopes are available for the production of radioconjugated anti-HCV antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example Tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as Tc^{99m} or I¹²³, Re¹⁸⁶ Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al. Cancer Research 52: 127-131 (1992); U.S. Pat. No. 5,208,020) may be used.

### VI. a-Interferon

The methods of combination therapy provided herein use, or incorporate, a-interferon. The terms “IFN-a” and “a-interferon” are used herein interchangeably.

IFN-a is a type I interferon produced by peripheral blood leukocytes or lymphoblastoid cells when exposed to live or inactivated virus, double-stranded RNA, or bacterial products. INF-a is the major interferon produced by virus-induced leukocyte cultures and, in addition to its pronounced antiviral activity, causes activation of NK cells.

A number of different subtypes exist. There are thirteen subtypes of a-interferon such as IFN-a1, IFN-a2, IFN-a4, IFN-a5, IFN-a6, IFN-a7, IFN-a8, IFN-a10, IFN-a13, IFN-a14, IFN-a16, IFN-a17, and IFN-a21. In some embodiments, the a-interferon is any of IFN-a1, IFN-a2, IFN-a4, IFN-a5, IFN-a6, IFN-a7, IFN-a8, IFN-a10, IFN-a13, IFN-a14, IFN-a16, IFN-a17, or IFN-a21. In some embodiments the a-interferon is IFN-a2. In some embodiments, IFN-a2 is any of IFN-a2a, IFN-a2b, or IFN-a2c. In some embodiments, the IFN-a2 is IFN-a2a. In some embodiments, the IFN-a2 is IFN-a2b. In some embodiments, the a-interferon is a derivative or variant of any of the a-interferons described above.

In some embodiments of any of the a-interferons described above, the a-interferon is formulated for extended or sustained release.

In some embodiments, the a-interferon is pegylated. In some embodiments, the pegylated a-interferon is pegylated IFN-a1, IFN-a2, IFN-a4, IFN-a5, IFN-a6, IFN-a7, IFN-a8, IFN-a10, IFN-a13, IFN-a14, IFN-a16, IFN-a17, or IFN-a21. In some embodiments, the pegylated a-interferon is pegylated IFN-a2. In some embodiments, pegylated IFN-a2 is any of pegylated IFN-a2a, pegylated IFN-a2b, or pegylated IFN-a2c. In some embodiments, the pegylated IFN-a2 is pegylated IFN-a2a. In some embodiments, the pegylated IFN-a2a is Pegasys®. In some embodiments, the pegylated IFN-a2 is pegylated IFN-a2b. In some embodiments, the pegylated IFN-a2b is Peg-Intron™.

In some embodiments, the a-interferon is any of Belerofon®, BLX-883 (Locteron™), Albuferon® (IFN-a2b), R7025 (Maxy-alpha), GEA0007.1-IFN-a variant.

### VII. Pharmaceutical Compositions

Pharmaceutical compositions useful in the present invention may comprise a therapeutically effective amount of the anti-HCV antibody and/or a-interferon and a pharmaceutically acceptable carrier, dilutent or excipient (including combinations thereof).

Pharmaceutical compositions may be for human or animal usage in human and veterinary medicine and will typically comprise any one or more of a pharmaceutically acceptable dilutent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The choice of pharmaceutical carrier, excipient or dilutent may be selected with regard to the intended route of administration and standard pharmaceutical practice. Pharmaceutical compositions may comprise as—or in addition to—the carrier, excipient or dilutent any suitable binder(s), lubricant(s), suspending agent(s), coating agent(s) or solubilizing agent(s).

Preservatives, stabilizers, dyes and even flavoring agents may be provided in pharmaceutical compositions. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be administered by a number of routes.

The humanized antibody may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g., as a carrier, dilutent or solubilizer. Alpha-, beta- and gamma-cyclodextrins are most commonly used and suitable examples are described in WO-A-91/11172, WO-A-94/02518 and WO-A-98/55148.

The pharmaceutical composition also can be incorporated, if desired, into liposomes, microspheres, or other polymer matrices. Liposomes, for example, which consist of phospholipids or other lipids, are nontoxic, physiologically acceptable and metabolizable carriers that are relatively simple to make and administer.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyethylene glycols, polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### VIII. Methods of Administration

The methods of treatment described herein comprising anti-HCV antibodies and a-interferon can be administered simultaneously (i.e., simultaneous administration), concurrently (i.e., concurrent administration), rotationally (i.e., rotationally administration), intermittently (i.e., intermittently administration) and/or sequentially (i.e., sequential administration).

In some embodiments, the anti-HCV antibodies and a-interferon are administered simultaneously. The term “simultaneous administration,” as used herein, means that the nanoparticle composition and the chemotherapeutic agent are administered with a time separation of no more than about 15 minute(s), such as no more than about any of 10, 5, or 1 minutes. When the drugs are administered simultaneously, the anti-HCV antibodies and a-interferon may be contained in the same composition (e.g., a composition comprising both the anti-HCV antibodies and a-interferon) or in separate compositions (e.g., anti-HCV antibodies are contained in one composition and the a-interferon is contained in another composition). In some embodiments, simultaneous administration of the anti-HCV antibodies and a-interferon can be combined with supplemental doses of the anti-HCV antibodies and a-interferon.

In some embodiments, the anti-HCV antibodies and a-interferon are administered sequentially. The term “sequential administration” as used herein means that the anti-HCV antibodies and a-interferon are administered with a time separation of more than about 15 minutes, such as more than about any of 20, 30, 40, 50, 60 or more minutes. Either the anti-HCV antibodies or a-interferon may be administered first. The anti-HCV antibodies and a-interferon are contained in separate compositions, which may be contained in the same or different packages.

In some embodiments, the administration of the anti-HCV antibodies and a-interferon are concurrent, i.e., the administration period of the anti-HCV antibodies and that of a-interferon overlap with each other. In some embodiments, the administration of the anti-HCV antibodies and a-interferon are non-concurrent. For example, in some embodiments, the administration of the anti-HCV antibodies is terminated before a-interferon is administered. In some embodiments, the administration of a-interferon is terminated before the anti-HCV antibodies is administered. The time period between these two non-concurrent administrations can range from about two days to one month, such as about one week.

The dosing frequency of the anti-HCV antibodies and a-interferon may be adjusted over the course of the treatment, based on the judgment of the administering physician. When administered separately, the anti-HCV antibodies and a-interferon can be administered at different dosing frequency or intervals. For example, the a-interferon composition can be administered weekly, while the anti-HCV antibodies can be administered more or less frequently. In some embodiments, sustained continuous release formulation of the anti-HCV antibodies and a-interferon may be used. Various formulations and devices for achieving sustained release are known in the art.

The anti-HCV antibodies and a-interferon can be administered using the same route of administration or different routes of administration.

The doses required for the anti-HCV antibodies and/or a-interferon may (but not necessarily) be lower than what is normally required when each agent is administered alone. Thus, in some embodiments, a subtherapeutic amount of the drug in the anti-HCV antibodies and a-interferon are administered. “Subtherapeutic amount” or “subtherapeutic level” refer to an amount that is less than the therapeutic amount, that is, less than the amount normally used when the anti-HCV antibodies and/or a-interferon are administered alone. The reduction may be reflected in terms of the amount administered at a given administration and/or the amount administered over a given period of time (reduced frequency).

In some embodiment, the administration of the combination of a humanized antibody and a second therapeutic agent ameliorates one or more symptom of HCV, reduces and/or suppresses viral titer and/or viral load, and/or prevents HCV more than treatment with the humanized antibody or second therapeutic agent alone.

In some embodiments, enough anti-HCV antibodies is administered so as to allow reduction of the normal dose of a-interferon required to effect the same degree of treatment by at least about any of 5%, 10%, 20%, 30%, 50%, 60%, 70%, 80%, 90%, or more. In some embodiments, enough a-interferon is administered so as to allow reduction of the normal dose of the anti-HCV antibodies required to effect the same degree of treatment by at least about any of 5%, 10%, 20%, 30%, 50%, 60%, 70%, 80%, 90%, or more.

The antibodies may be administered, for example, in the form of immune serum or may more preferably be a purified recombinant or monoclonal antibody. Methods of producing sera or monoclonal antibodies with the desired specificity are routine and well-known to those skilled in the art. One skilled in the art understands that the antibody/ies can be administered by various routes including, for example, injection, intubation, via a suppository, orally or topically, the latter of which can be passive, for example, by direct application of an ointment or powder containing the antibodies, or active, for example, using a nasal spray or inhalant. The antibodies can also be administered as a topical spray, if desirable, in which case one component of the composition is an appropriate propellant.

The humanized antibodies and fragments thereof described herein can be administered to a subject in accord with known methods, such as by intravenous administration, e.g., as a bolus or by continuous infusion over a period of time, by subcutaneous, intramuscular, intraperitoneal, intracerobrospinal, intrasynovial, intrathecal, inhalation routes, intravenous, intra-arterial, intraperitoneal, intrapulmonary, oral, inhalation, intravesicular, intra-tracheal, subcutaneous, intraocular, or transdermal, generally by intravenous or subcutaneous administration.

In some embodiments, the administered anti-HCV antibodies are substantially purified (e.g., preferably at least 95% homogeneity, more preferably at least 97% homogeneity, and most preferably at least 98% homogeneity, as judged by SDS-PAGE).

Suitably, a passive immunization regime may conveniently comprise administration of the humanized antibody of fragment thereof as described herein and/or administration of antibody in combination with other antiviral therapeutic compounds. Recently such passive immunization techniques have been used safely to treat HIV infection (Armbruster et al, J. Antimicrob. Chemother. 54, 915-920 (2004); Stiegler & Katinger, J. Antimicrob. Chemother. 51, 757-759 (2003)).

The active or passive immunization methods of the invention should allow for the protection or treatment of individuals against infection with viruses of any of genotypes 1-6 of HCV, except for very occasional mutant isolates (such as that exemplified by UKN5.14.4, below) which contain several amino acid differences to that of the consensus peptide epitope defined above.

As will be understood by those of ordinary skill in the art, the appropriate doses of a-interferon will be approximately those already employed in clinical therapies wherein a-interferon is administered alone or in combination with other anti-viral compounds. Variation in dosage will likely occur depending on the condition being treated. As described above, in some embodiments, a-interferon may be administered at a reduced level.

In some embodiments of any of the methods provided herein, the dosing frequency of the anti-HCV antibody and/or a-interferon includes, but is not limited to, twice weekly, three times weekly, weekly without break; weekly, three out of four weeks; once every three weeks; once every two weeks; or two out of three weeks.

In some embodiments of any of the methods provided herein, the dosage of a-interferon is between about any of 10-500 μg, 20-250 μg, or 40-200 μg. In some embodiments, a-interferon is administered subcutaneous, intramuscular, intraperitoneal, intracerobrospinal, intrasynovial, intrathecal, inhalation routes, intravenous, intra-arterial, intraperitoneal, intrapulmonary, oral, inhalation, intravesicular, intra-tracheal, subcutaneous, intraocular, or transdermal, generally by subcutaneous administration.

In some embodiments, the anti-HCV antibody and/or a-interferon is administered in a therapeutic effective amount to effect beneficial clinical results, including, but not limited to ameliorating one or more symptoms of HCV infections or aspects of HCV infection. In some embodiments, the anti-HCV antibody and/or a-interferon is administered in a therapeutic effective amount to reduce viral titer and/or viral load of HCV.

### IX. Diagnosis

In yet a further aspect, there is provided a diagnostic test apparatus and method for determining or detecting the presence of HCV in a sample. The apparatus may comprise, as a reagent, one or more humanized antibodies as described herein. The antibody/ies may, for example, be immobilized on a solid support (e.g., on a microtiter assay plate, or on a particulate support) and serve to “capture” HCV particles from a sample (e.g., a blood or serum sample or other clinical specimen—such as a liver biopsy). The captured virus particles may then be detected by, for example, adding a further, labeled, reagent which binds to the captured virus particles. Conveniently, the assay may take the form of an ELISA, especially a sandwich-type ELISA, but any other assay format could in principle be adopted (e.g., radioimmunoassay, Western blot) including immunochromatographic or dipstick-type assays.

For diagnostic purposes, the humanized antibodies as described herein may either be labeled or unlabelled. Unlabelled antibodies can be used in combination with other labeled antibodies (second antibodies). Alternatively, the antibodies can be directly labeled. A wide variety of labels may be employed—such as radionuclides, fluors, enzymes, enzyme substrates, enzyme cofactors, enzyme inhibitors, ligands (particularly haptens), etc. Numerous types of immunoassays are available and are well known to those skilled in the art.

Since the humanized antibodies as described herein can bind to HCV from any of genotypes 1-6, the assay apparatus and corresponding method should be capable of detecting in a sample HCV representative from any of these genotypes.

In some embodiments, the sample is compared to a control sample. In some embodiments, the control sample is from an individual known to be infected with HCV. In some embodiments, the individual is known to infected with one or more HCV genotypes selected from the group consisting of genotype 1 (e.g., genotype 1a and genotype 1b), genotype 2 (e.g., genotype 2a, genotype 2b, genotype 2c), genotype 3 (e.g., genotype 3a), genotype 4, genotype 5, and genotype 6. In some embodiments, the control sample is from an individual known not to be infected with HCV.

In some embodiments, any of the methods of treatment described are based on the determination or detection of HCV in a sample by any of the anti-HCV antibodies described herein. As used herein, “based upon” includes (1) assessing, determining, or measuring the subject's characteristics as described herein (and preferably selecting a subject suitable for receiving treatment); and (2) administering the treatment(s) as described herein.

In some embodiments a method is provided for identifying an individual suitable or not suitable (unsuitable) for treatment with the anti-HCV antibodies and a-interferon.

### X. Kits and Articles of Manufacture

Kits can also be supplied for use with the anti-HCV antibodies and a-interferon in the protection against or detection of a cellular activity or for the presence of a selected antigen. Thus, the anti-HCV antibodies and a-interferon may be provided, usually in a lyophilized form in a container, either alone or in conjunction with additional antibodies specific for the desired cell type.

The antibodies, which may be conjugated to a label or toxin, or unconjugated, are included in the kits with buffers, such as Tris, phosphate, carbonate, etc., stabilizers, biocides, inert proteins, e.g., serum albumin, or the like. Generally, these materials will be present in less than about 5% wt. based on the amount of antibody, and usually present in total amount of at least about 0.001% wt. based again on the antibody concentration. Frequently, it will be desirable to include an inert extender or excipient to dilute the active ingredients, where the excipient may be present in from about 1 to 99% wt. of the total composition.

The invention also provides diagnostic kits, for example, research, detection and/or diagnostic kits. Such kits typically contain the anti-HCV antibodies as described herein. Suitably, the antibody is labeled or a secondary labeling reagent is included in the kit. Preferably, the kit is labeled with instructions for performing the intended application, for example, for performing an in vivo imaging assay.

In some embodiments, any of the kits described herein contain a package insert. Package insert refers to instructions customarily included in commercial packages of therapeutic products, which contain information about the indications, usage, dosage, administration, contraindications and/or warnings concerning the use of such therapeutic products. In one embodiment, the package insert indicates that the composition may be used in any of the methods of combination therapy described herein.

The anti-HCV antibodies and a-interferon can be present in separate containers or in a single container. It is understood that the kit may comprise one distinct composition or two or more compositions wherein one composition comprises anti-HCV antibodies and one composition comprises a-interferon.

Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Provided herein are articles of manufacture which comprise anti-HCV antibodies and a-interferon described herein. In some embodiments, the articles of manufacture comprise a container and a label or package insert on or associated with the container. The anti-HCV antibodies and a-interferon can be present in separate containers or in a single container. It is understood that the article of manufacture may comprise one distinct composition or two or more compositions wherein one composition comprises anti-HCV antibodies and one composition comprises a-interferon.

### XI. General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

The invention will now be further described by way of Examples, which are meant to serve to assist one of ordinary skill in the art in carrying out the invention and are not intended in any way to limit the scope of the invention.

### EXAMPLES

The examples, which are intended to be purely exemplary of the invention and should therefore not be considered to limit the invention in any way, also describe and detail aspects and embodiments of the invention discussed above. The foregoing examples and detailed description are offered by way of illustration and not by way of limitation.

### Example 1

### Materials and Methods

Cloning of Humanized V genes

The heavy chain V regions (see Example 2) were cloned into pG1D200 via HindIII and ApaI restriction enzyme sites. Similarly, the light chain V regions were cloned into pKN100 via the HindIII and BamHI sites. pG1D200 vector were prepared for ligation by digesting 5 μg of DNA with 20 units of HindIII and ApaI in multicore (Promega) restriction digest buffer for 2 hrs at 37° C. Then 1 unit of shrimp alkaline phosphatase was added for 30 min at 37° C. and inactivated at 65° C. for 20 minutes. The vector preparation was then purified on a Qiaquick (Qiagen) column following manufacturer's instructions. The vector was eluted in 50 μl Similarly pKN100 vector was prepared by digesting 5 μg of DNA with 20 units of HindIII and BamHI in buffer E (Promega) for 1 hour at 37° C. The DNA was treated with shrimp alkaline phosphatase and purified as described above. V region DNA including mutant V regions was supplied by GENART in the vectors pGA4 or pGA1. Insert DNAs (approx 4 ug) were digested as described above and the heavy and light chain fragments were purified from the vector by gel electrophoresis. The appropriate band was excised from the gel and purified on a Qiaquick column (Qiagen) and eluted in 50 μl following manufacturer's instructions. Ligations were carried out by mixing 1 μl of vector with either 1 or 3 μl of insert DNA in 1× ligase buffer (Promega) and 10 units of ligase (Promega). The reaction was incubated at 14° C. overnight and 2.5 ml were used to transform 50 ml of DH5a competent cells (Invitrogen).

Site Directed Mutagenesis

Site directed mutagenesis was carried out by outsourcing the mutagenesis to GENEART AG except for the chimeric heavy chain mutants AP33 Y47W and Y47F. The chimeric heavy chain mutagenesis was carried out using the following oligonucleotides:

(SEQ ID NO: 189)
AP33_Y47F_F: AATAAACTTGAGTTCATGGGATACATAAGT
(SEQ ID NO: 190)
AP33_Y47F_R: ACTTATGTATCCCATGAACTCAAGTTTATT
(SEQ ID NO: 191)
AP33_Y47W_F: GAATAAACTTGAGTGGATGGGATACATAAG
(SEQ ID NO: 192)
AP33_Y47W_R: CTTATGTATCCCATCCACTCAAGTTTATTC.

The mutagenesis PCR reaction used oligonucleotides at a final concentration of 0.5 micro Molar, combined with 20 ng of VH.pG1D200 (Chimeric heavy chain construct) and 1× Fusion master mix (NEB). PCR conditions were: 98° C. for 30 sec then 12 cycles of 98° C. for 10 sec, 55° C. for 15 sec, 72° C. for 2 min 15 sec. Once the PCR reaction was complete, 20 units of DpnI were added to each PCR reaction for 1 hour at 37° C. 2 μl of the PCR digest mixture was used to transform 50 μl of XL-1 blue competent cells (Stratagene).

The recombinant chimeric and humanized heavy chains RHA, RHbcdefgh (RHb-h) and humanized light chains RKA and RK2bc were cloned into the antibody expression vectors pG1D200 and pKN100 respectively. Plasmid DNA was prepared using the appropriate Qiagen plasmid purification kit.

Electroporation

Cos7 cells were grown and split 1:3 on the day before transfection. Log phase Cos7 cells were trypsinised and washed in PBS and resuspended at 10⁷ cells/ml in PBS and 700 μl of cells aliquoted into electroporation cuvettes (Bio-Rad). 5 μg each of heavy and light chain constructs were mixed with the cells and electroporated at 1.9 KV and 25 μF. Cells were left for 10 minutes at room temperature to recover and added to 8 ml of DMEM with Glutamax (Invitrogen)/10% FCS/Penicillin 500 U/ml/Streptomycin 500 μg/ml on 10 cm2 tissue culture plates. The supernatant was harvested after 3 days and antibody concentration was analyzed by ELISA.

IgG1 ELISA

Maxisorp plates were coated with 0.4 μg/ml goat anti-human IgG antibody and stored at 4° C. for no more than 1 month. Before use, plates were washed three times in PBS/0.02% Tween 20 (v/v) then blocked in PBS/0.02% Tween 20 (v/v)/0.2% (w/v) BSA. Plates were washed as before and sample supernatant added over a concentration range using doubling dilutions and incubated at 37° C. for 1 hr. Plates were washed as before and incubated with goat anti-human kappa light chain peroxidase conjugate (Sigma) at 1:5000 dilution. Plates were washed, as before, then 150 μL of K Blue One-Step substrate Neogen) was added. After 10 minutes the reaction was stopped with 50 μL of Red Stop solution (Neogen) and the optical density was measured at 655 nm.

Peptide ELISA

ELISA plates (Nunc Maxisorp) were coated with streptavadin (Sigma S0677) (10 μg/ml in 100 mM Na₂HPO₄, 50 mM citric acid pH5.0, 100 μl/well) and stored at 4° C. for up to one month. Before use, plates were washed three times in PBS/0.1% (v/v) Tween 20 and blocked with 200 μl of PBS/2% BSA (w/v) for one hour at 37° C. The plates were then washed as before and 100 μl peptide (0.5 μg/ml in SEC buffer) was added for one hour at 37° C. All peptides included the biotinylated linker sequence GSGK-biotin. The plate was washed as before and 100 μl antibody supernatant added in serial doubling dilutions in SEC buffer and incubated for one hour. Plate was washed as before and incubated for one hour at 37° C. with HRP conjugated anti-human kappa antibody (Sigma) at 1:5000 dilution (100 μl/well). The plates were washed as before and 150 μl for TMB One-Step K-Blue substrate (Neogen) added for 10 minutes and stored in the dark at room temperature. The reaction was stopped with 50 μl of Red Stop (Neogen). The optical density was measured at 655 nm.

Preparation of Antibody for HCVpp Infection Assays

In order to carry out HCVpp experiments, antibodies from COS7 cell transfection supernatants were purified and concentrated by Protein A purification. For each chimeric or humanized antibody: Prosep-vA beads (Millipore) were resuspended and 400 μl added to a 10 ml disposable chromatography column (Pierce) and washed with 20 ml of PBS. COS7 transfection supernatant (approximately 150 ml) was added to the column under gravity flow. The column was subsequently washed with PBS (20 ml) and eluted with 0.5 ml of Immunopure IgG Elution buffer (Pierce). The eluate was neutralized with 20 μl of 1 M Tris/HCL pH 7.6 and dialyzed in a 0.5 ml Slide-A-Lyser (Pierce) in 3 liters of PBS overnight at 4° C.

HCV Pseudoparticle Infection Assays

HCVpp for genotypes 1, 2, 3, 4, and 6 were made by transfecting HEK cells with plasmids encoding HCV glycoprotein sequences, MLV gag-pol and luciferase reporter, then the conditioned medium was concentrated and partially purified by ultracentrifugation through a cushion of 20% sucrose. See Owsianka A. et al., J Virol 79:11095-104 (2005). The HCVpp for genotype 5 was made in a similar way except that the partial purification step through a sucrose gradient was omitted since adversely affected infectivity of the genotype 5 pseudoparticles. Three-fold dilutions of each antibody in cell culture medium were mixed with HCVpp and the antibody/HCVpp mixtures were incubated at 37° C. for 1 h, and then added to human hepatomaHuh-7 target cells in triplicate wells. After 4 h incubation at 37° C., the inoculum was removed and replaced with fresh medium. After 3 days the cells were lysed and assayed for luciferase activity. Multiple wells were infected with HCVpp in the absence of antibody, and all the results are expressed as a percentage of this “no antibody” control. The genotypes for HCV used in the following experiments are shown in Table 2.

TABLE 2
The genotypes of HCV and the IC50 and IC90s of the chimeric and
humanized antibodies.
Experiment 1

| | Genotype | | | | | |
|---|---|---|---|---|---|---|
| IC units | 1a H77 20 | | 2a JFH1 | | 3a F4/2-35 | |
| (μg/ml) | IC50 | IC90 | IC50 | IC90 | IC50 | IC90 |
| Vh/V1 | <0.137 | 0.31 | 1.1 | 9.31 | 0.48 | 3.2 |
| RH B- | <0.41 | 0.73 | 2.7 | 22.1 | 1.15 | 8.3 |
| H/RK2b | | | | | | |
| RH-C/RK2b | <0.41 | <0.41 | 0.64 | 7 | <0.41 | 2.15 |
| RH-H/RK2b | <0.41 | 1 | 3 | 26 | 0.67 | 8.3 |

Experiment 2

| | Genotype | | | | | | | |
|---|---|---|---|---|---|---|---|---|
| IC units | 1a H77 20 | | 2A2.4 | | 4.21.16 | | 6.5.8 | |
| (μg/ml) | IC50 | IC90 | IC50 | IC90 | IC50 | IC90 | IC50 | IC90 |
| RH B- | 0.08 | 1 | 1.7 | 20 | 0.33 | 3.64 | <0.41 | 3.7 |
| H/RK2b | | | | | | | | |
| RH-C/ | <0.0137 | 0.24 | 0.51 | 6 | <0.41 | 0.92 | <0.41 | 1.8 |
| RK2b | | | | | | | | |
| RH-H/ | 0.06 | 1.2 | 3.33 | 26.67 | 0.33 | 5.3 | 0.79 | 18 |
| RK2b | | | | | | | | |
| Vh/V1 | 0.018 | 0.28 | 0.88 | 6.67 | <0.137 | 0.83 | <0.137 | 3.7 |

Experiment 3

| | Genotype | | | | | | | |
|---|---|---|---|---|---|---|---|---|
| IC units | 1a H77 20 | | 1A20.8 | | 1B5.23 | | 2B1.1 | |
| (μg/ml) | IC50 | IC90 | IC50 | IC90 | IC50 | IC90 | IC50 | IC90 |
| Vh/V1 | 0.03 | 0.43 | 1.1 | 11.5 | 1.15 | 11 | 3 | >15 |
| RH-C/RK2b | 0.032 | 0.6 | 1.6 | 15 | 0.9 | 8.3 | 3 | >15 |

Experiment 4

| | |
|---|---|
| | Genotype |
| IC units | 5.15.11 |

| (μg/ml) | IC50 | IC90 |
|---|---|---|
| Vh/V1 | 0.088 | 1.11 |
| RH-C/RK2b | 0.053 | 0.82 |
| RH-H/RK2b | 0.37 | 8 |

### Results

### Example 1A

### Selection of a Leader Sequence for AP33RHA

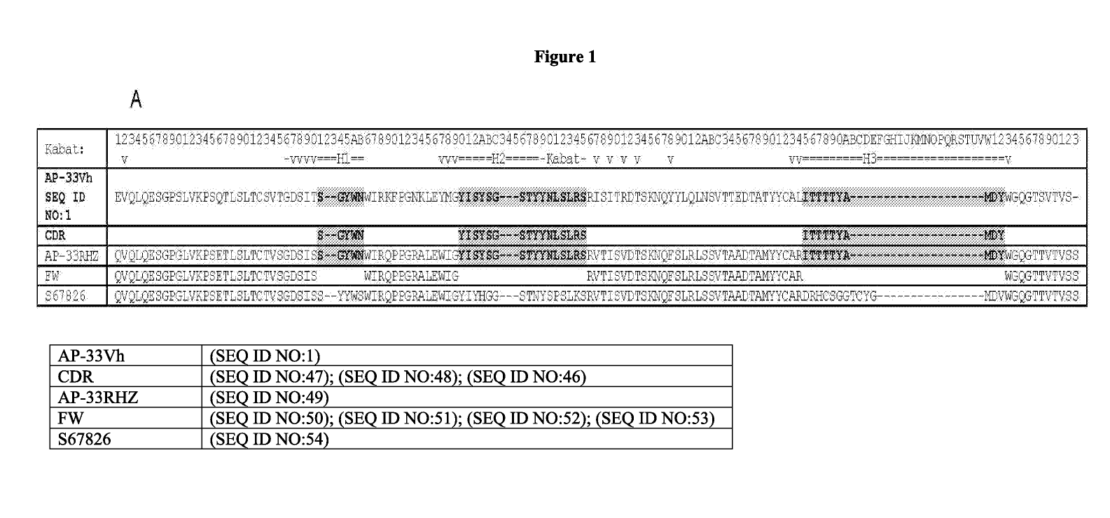

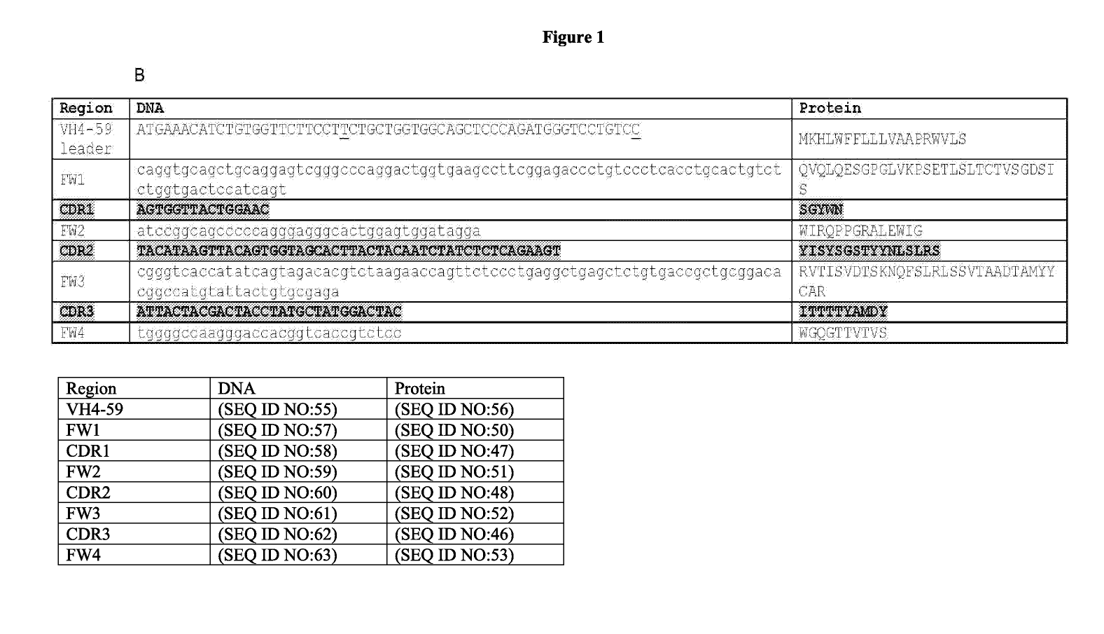

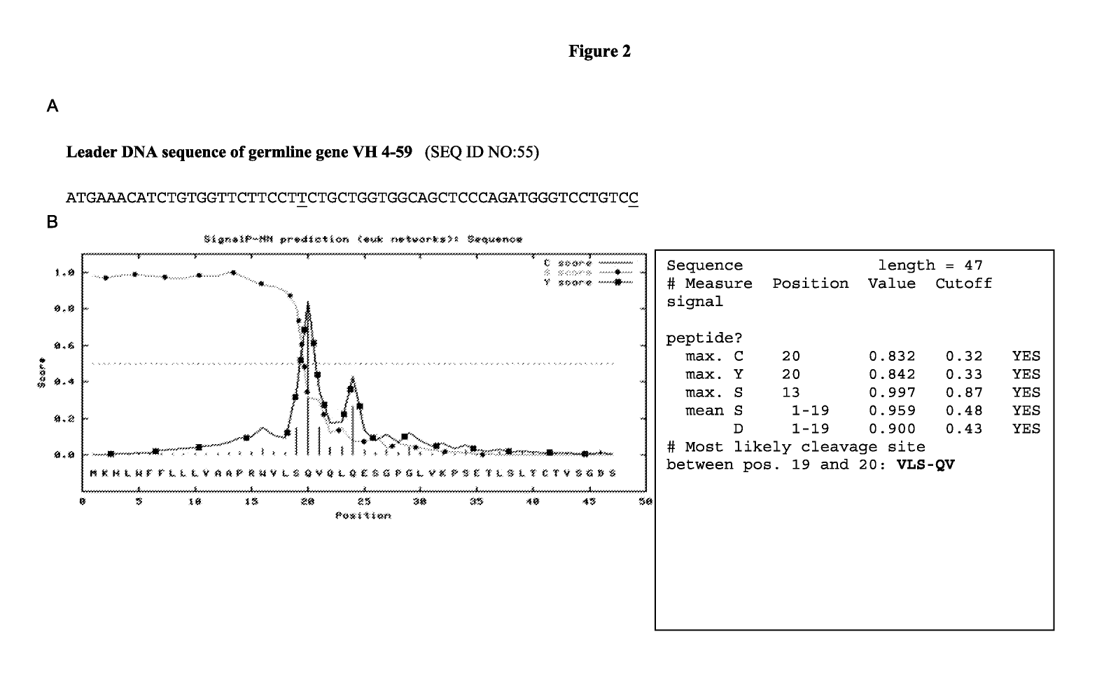

The initial humanization is the graft of the Kabat CDRs 1, 2 and 3 from AP33VH into the acceptor 567826 Kabat FWs 1, 2, 3, 4 (FIG. 1). This sequence requires the addition of a signal peptide from the germline gene VH4-59 that has the closest sequence identity to S67826 (FIG. 2). We used the SignalP (Foote J. and Winter G., J Mol Biol 224:487-99 (1992)) (V2.0.b2) server to confirm that this leader (FIG. 2) would cut with signal peptidase when preceding the S67826 FW1 sequence. FIG. 1 shows the generation of AP33RHA protein and DNA sequence by intercalating the AP33 CDRs into the human FW. The DNA sequence of AP33RHA including its leader is shown below:

(SEQ ID NO: 193)
ATGAAACATCTGTGGTTCTTCCTTCTGCTGGTGGCAGCTCCCAGATGGG
TCCTGTCCcaggtgcagctgcaggagtcgggcccaggactggtgaagcc
ttcggagaccctgtccctcacctgcactgtctctggtgactccatcagt
AGTGGTTACTGGAACatccggcagcccccagggagggcactggagtgga
taggaTACATAAGTTACAGTGGTAGCACTTACTACAATCTATCTCTCAG
AAGTcgggtcaccatatcagtagacacgtctaagaaccagttctccctg
aggctgagctctgtgaccgctgcggacacggccatgtattactgtgcga
gaATTACTACGACTACCTATGCTATGGACTACtggggccaagggaccac
ggtcaccgtctcc

AP33RHA DNA sequence with leader. Italics, upper case text indicates leader sequence, lower case text indicates FW, and upper case, bolded text indicates CDR sequences.

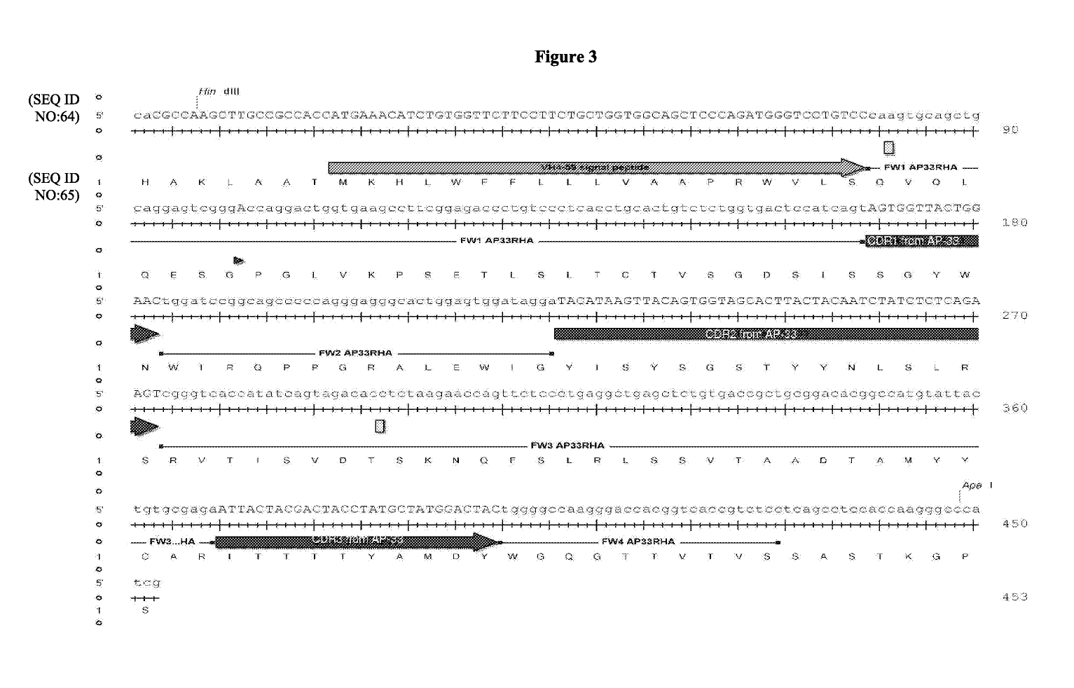

The completed protein and DNA sequence of the AP33RHA including the VH4-59 signal peptide is shown in FIG. 3.

### Example 1B

### Generation of AP33RKA, AP33RK2, AP33RK3 and AP33 RK4 Sequences

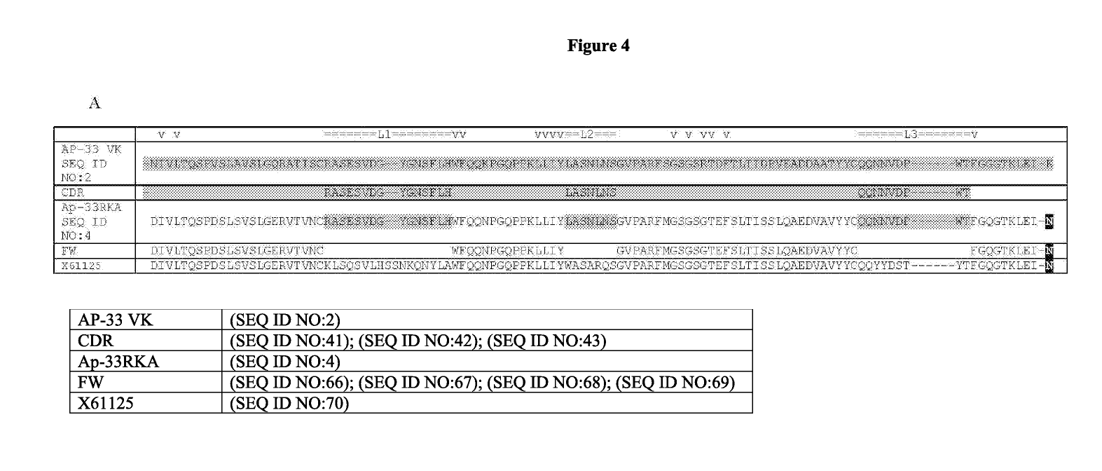

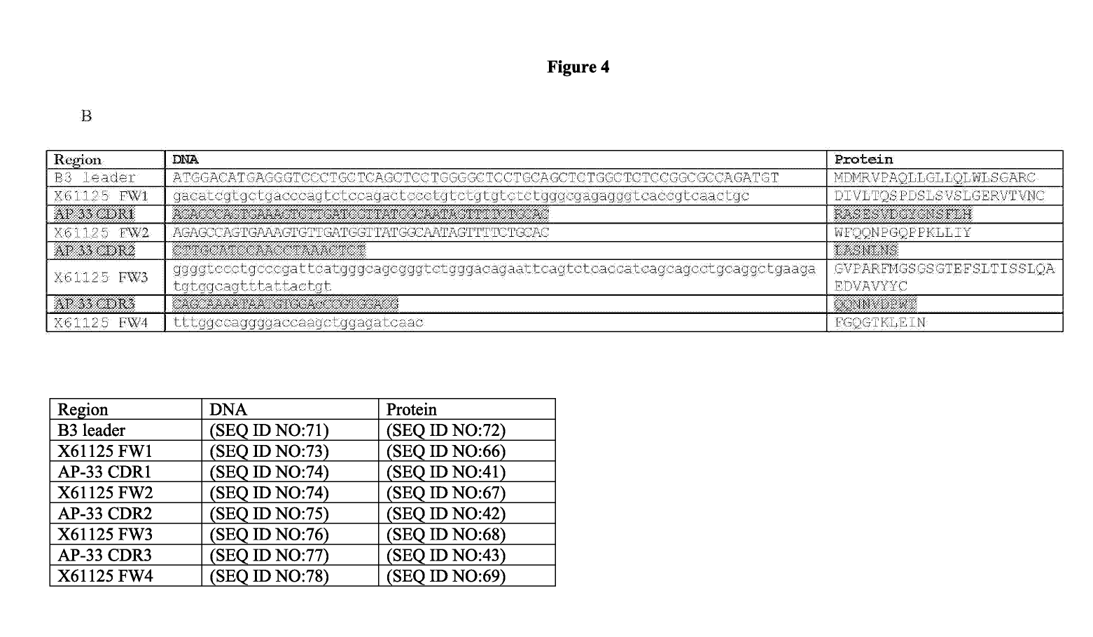

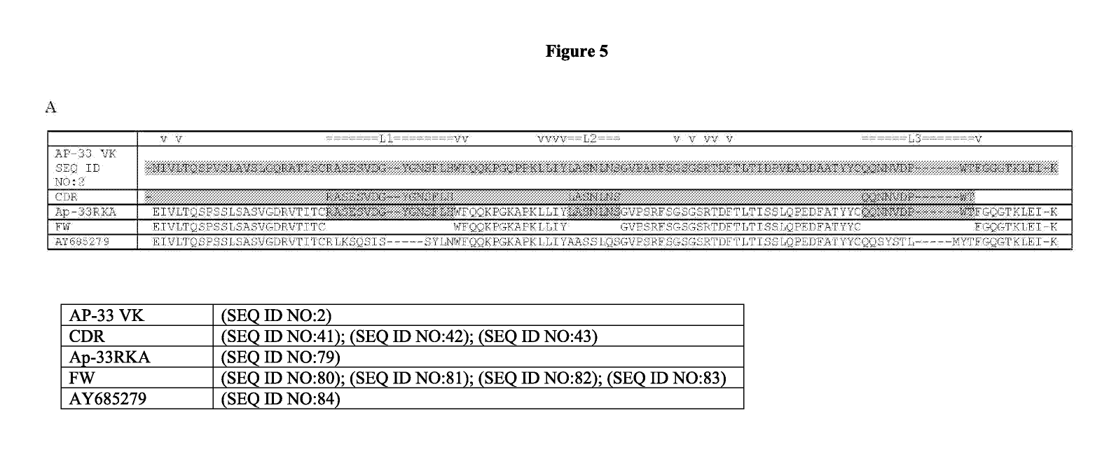

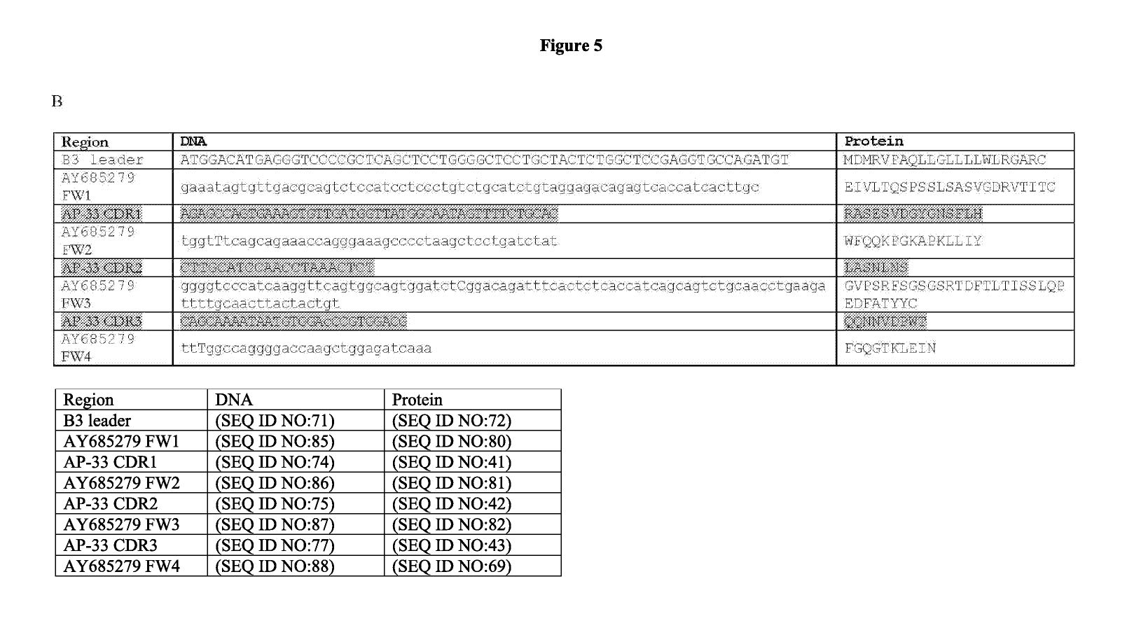

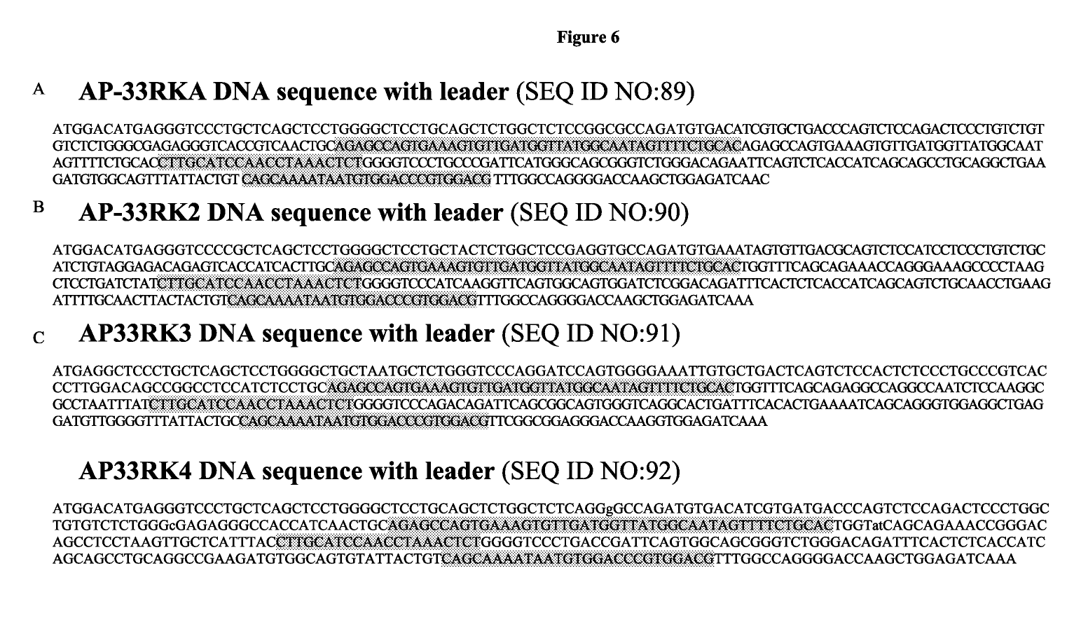

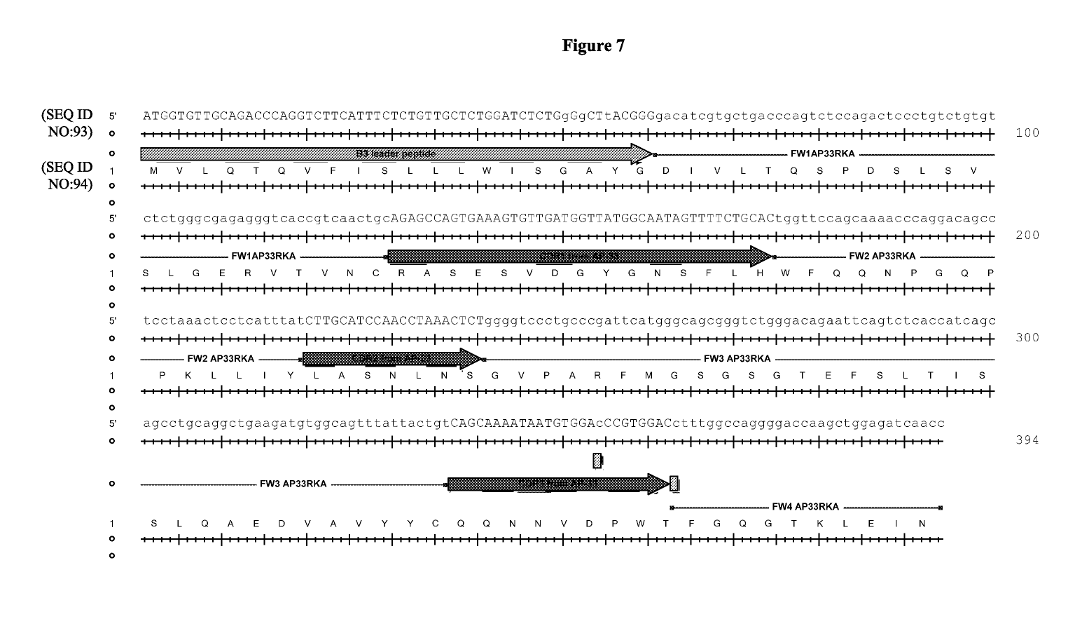

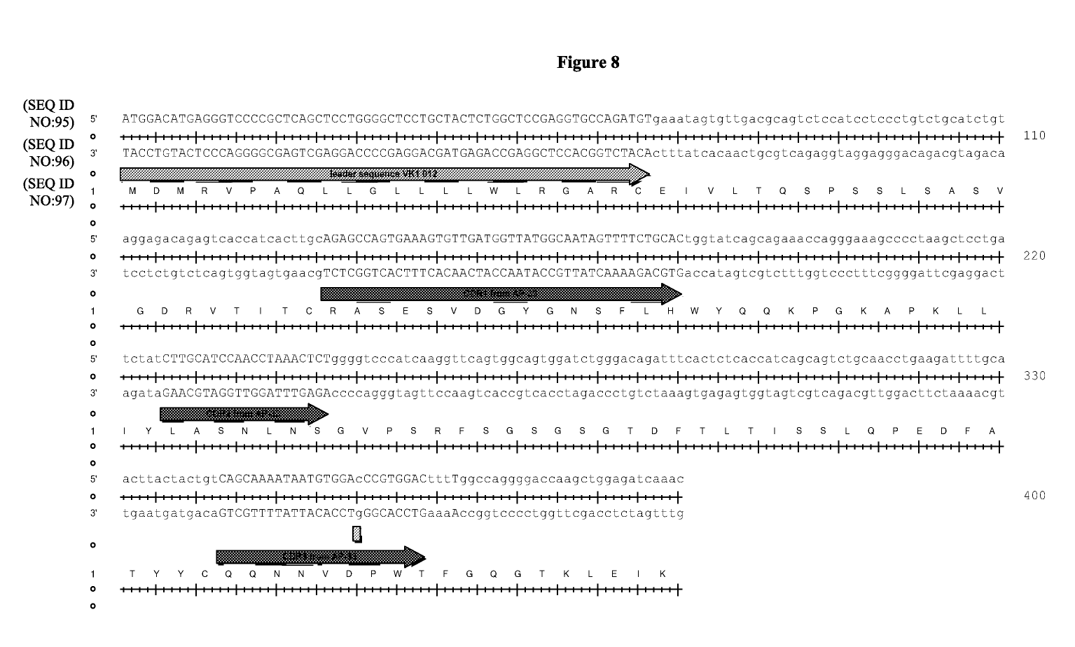

Intercalating the protein and DNA sequences of AP33VK CDRs between FWs 1, 2, 3 and 4 of X611251 is shown in FIG. 4, together with the DNA sequence of the B3 leader (AP33RKA). Intercalating the protein and DNA sequences of AP33VK CDRs into FWs 1, 2, 3 and 4 of AY685279 is shown in FIG. 5, together with the DNA sequence of the VKI-012/02. The complete AP33RKA and AP33RK2 sequence with their respective leader sequences attached is shown in FIGS. 6-8.

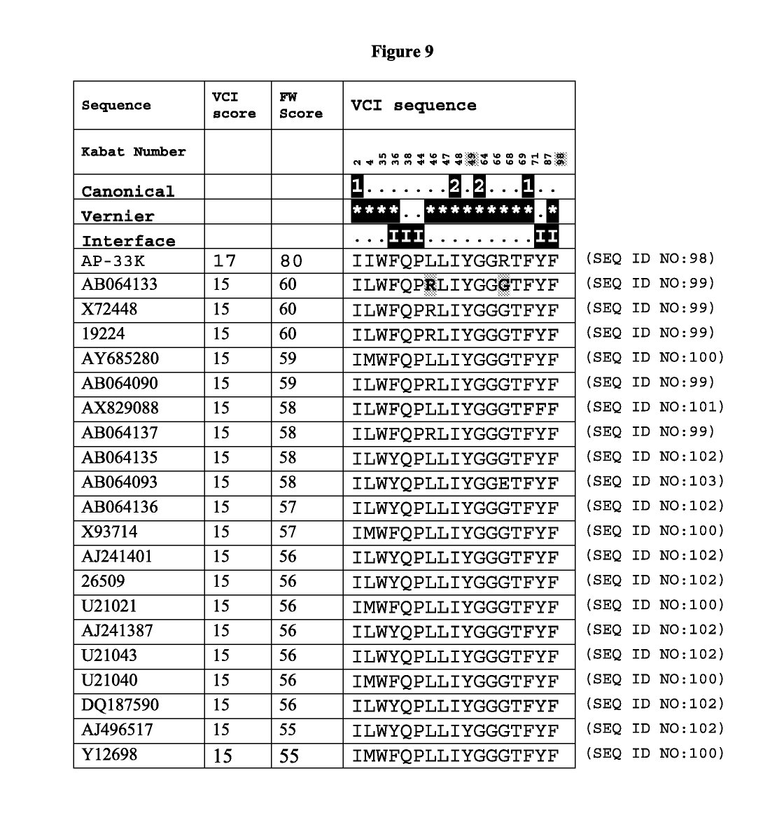

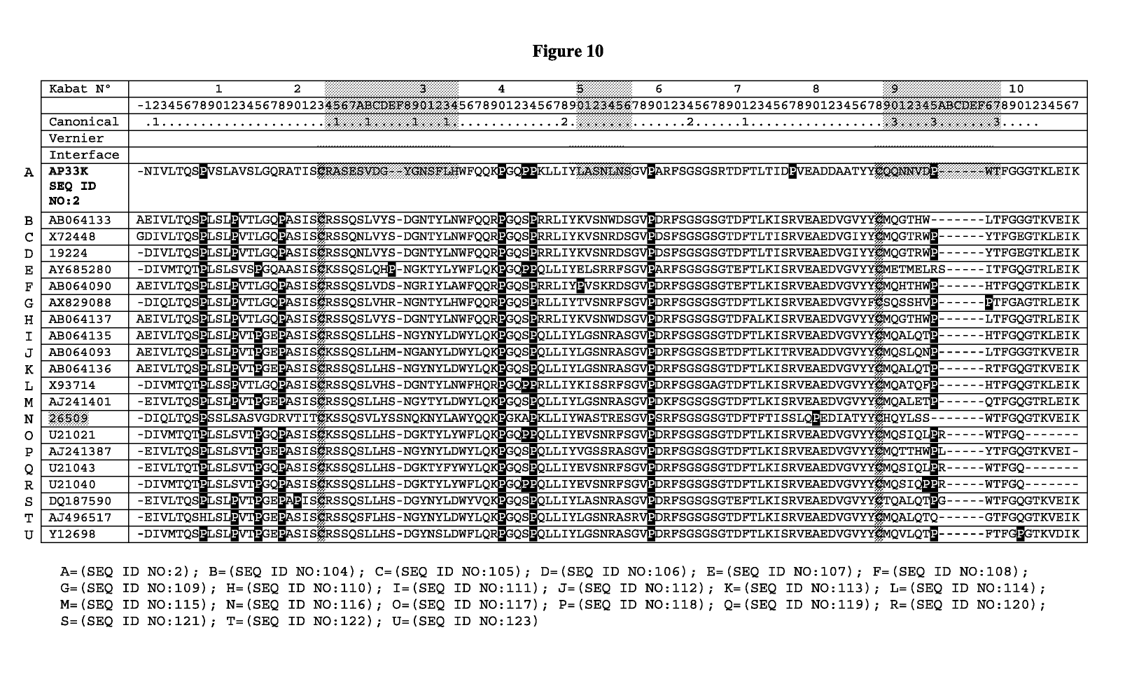

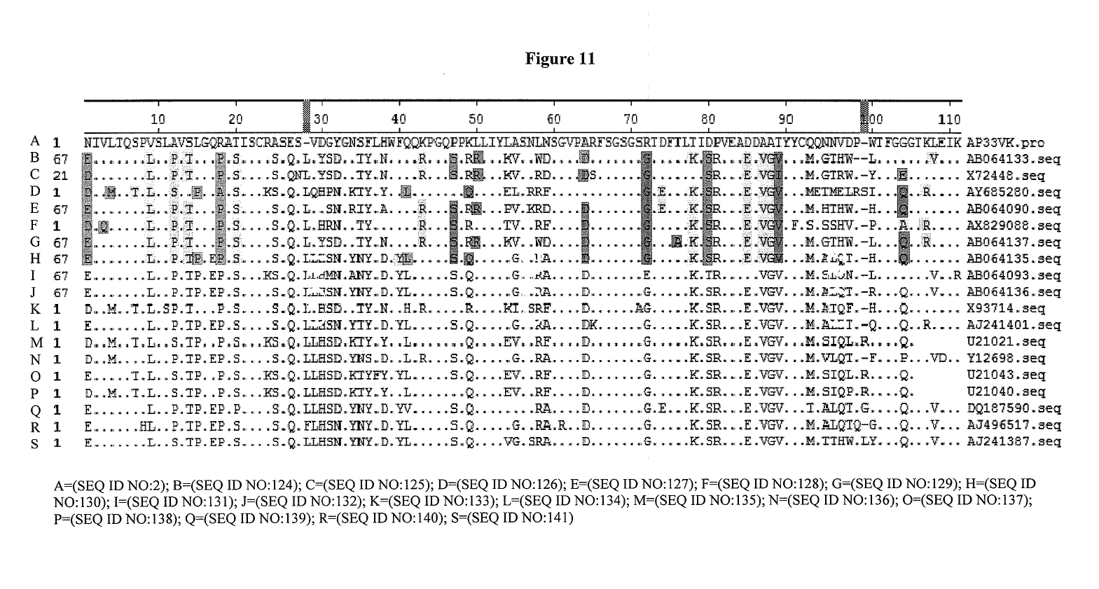

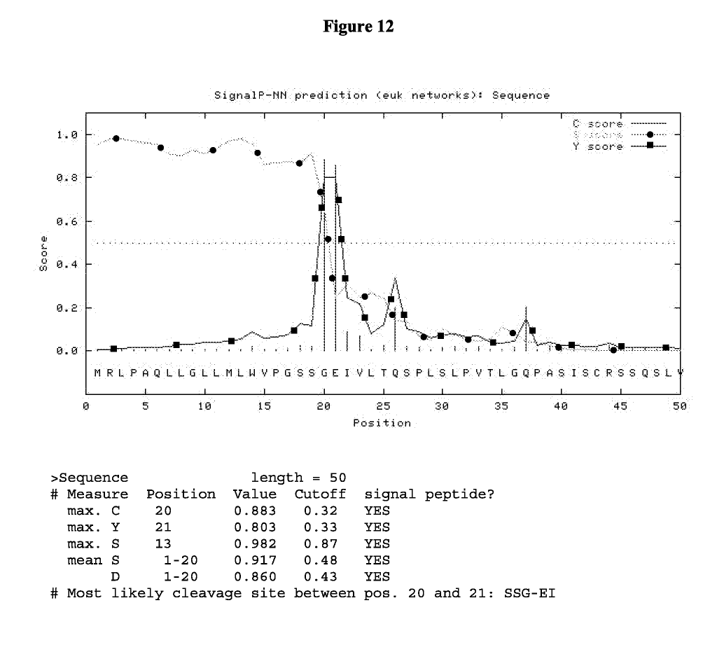

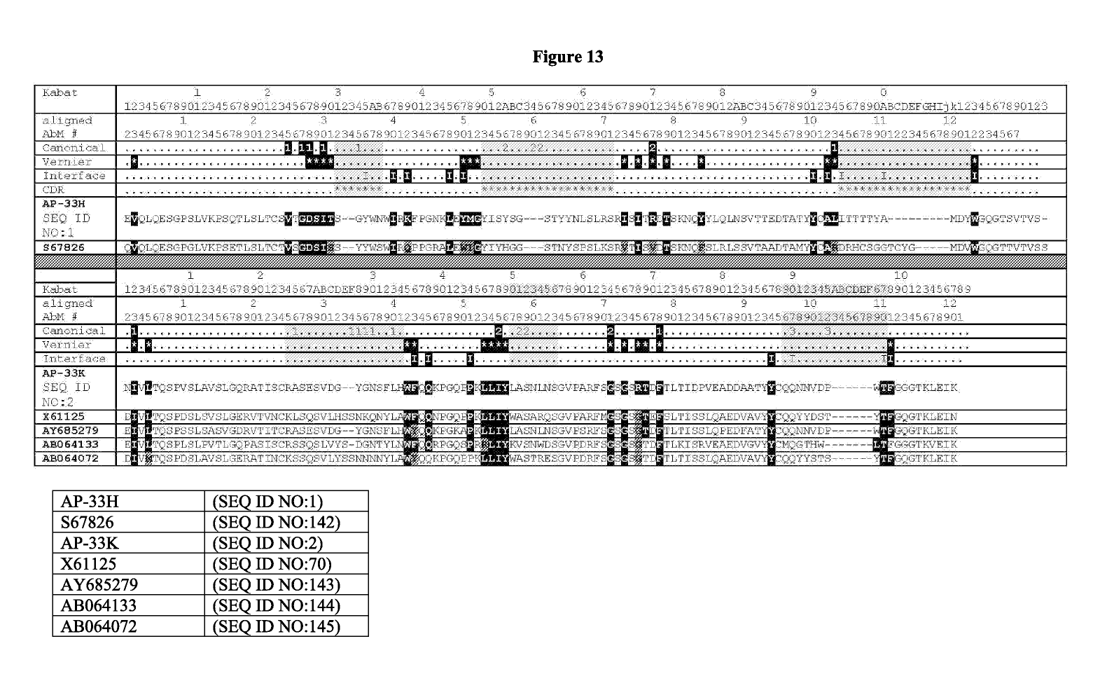

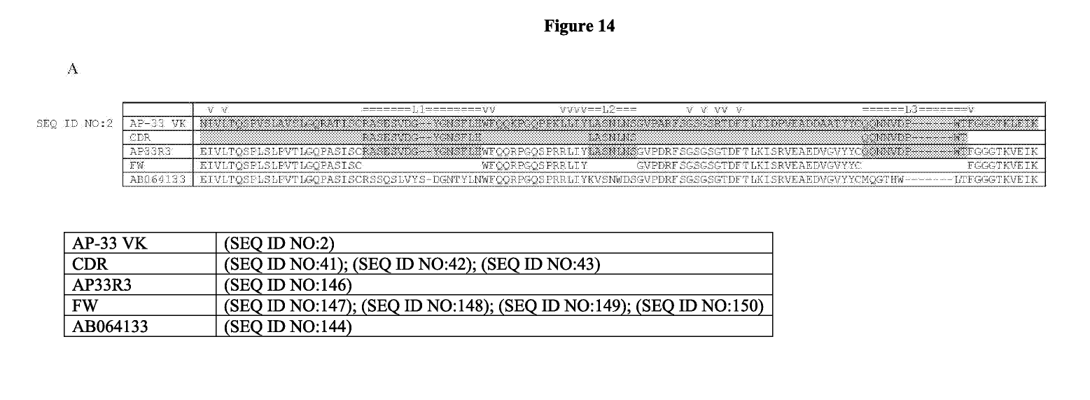

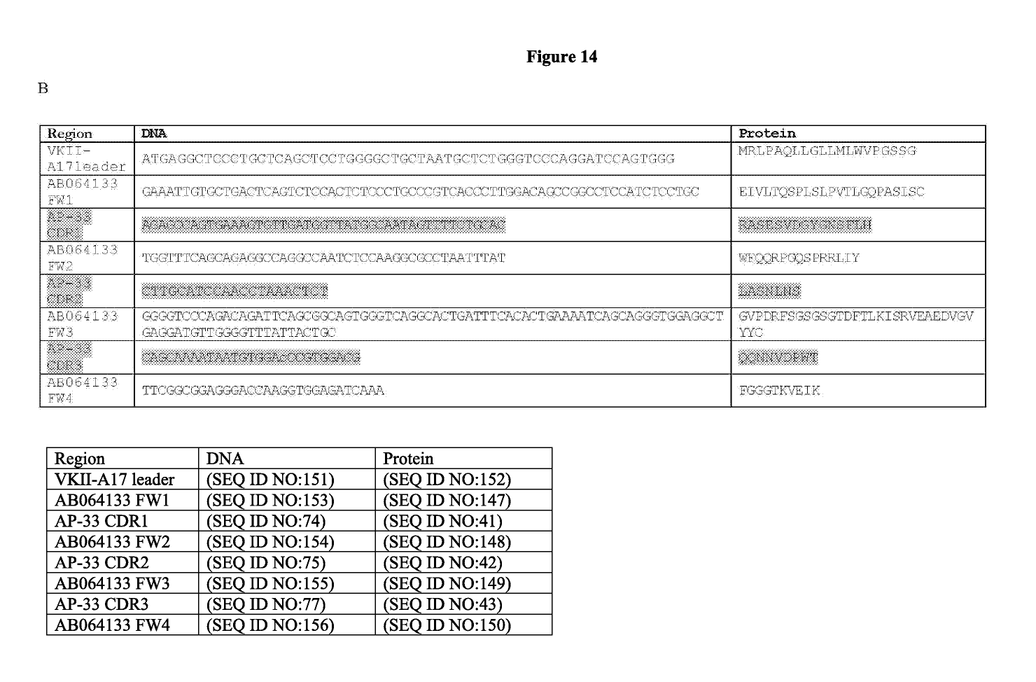

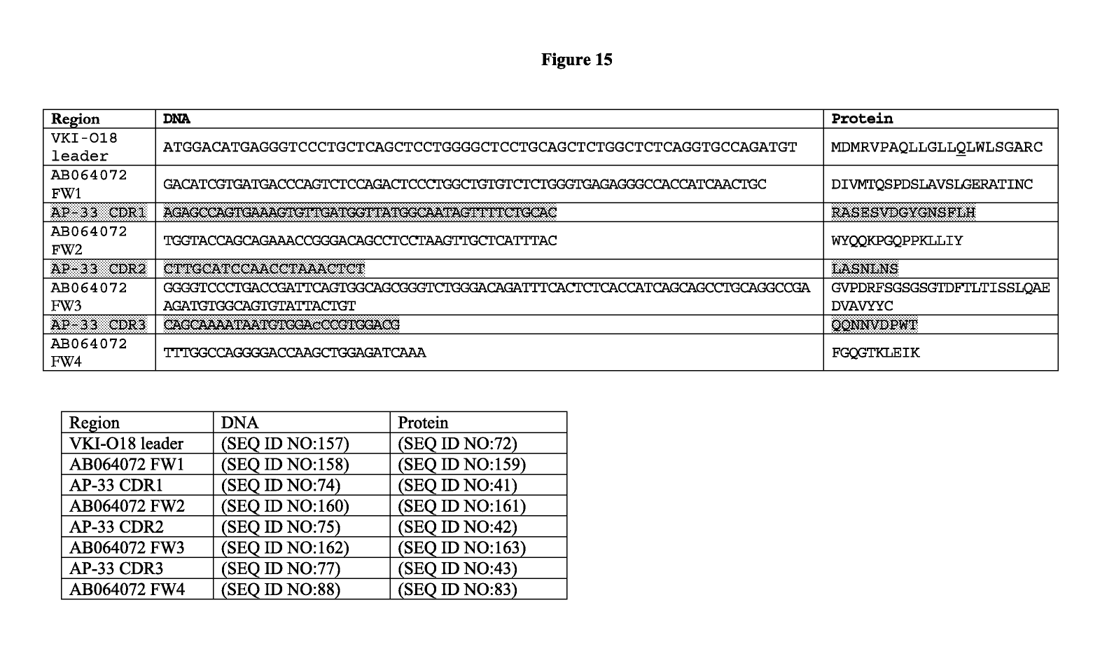

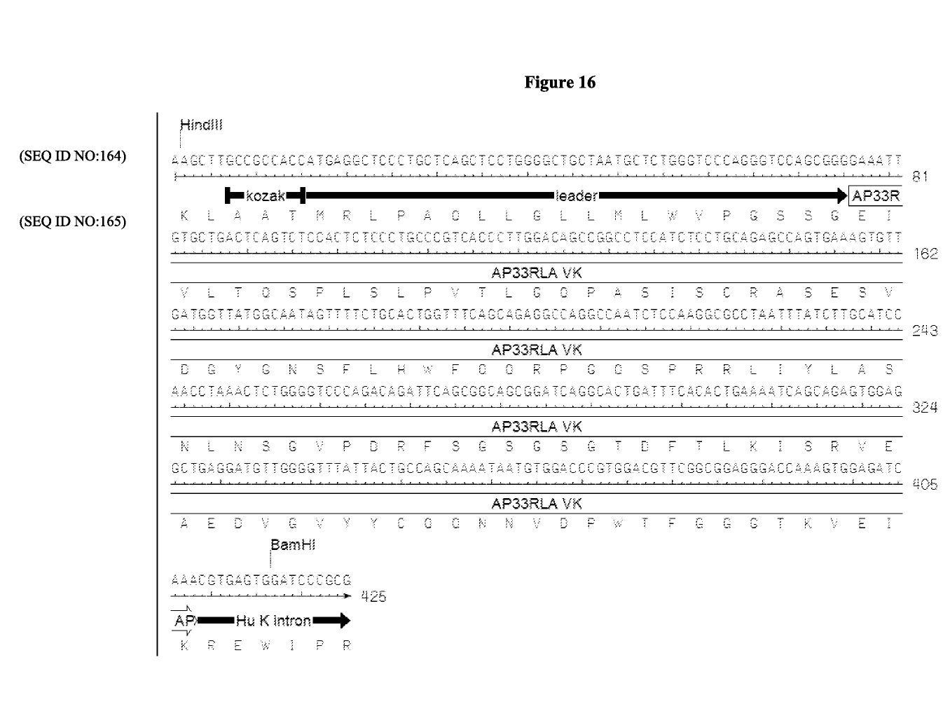

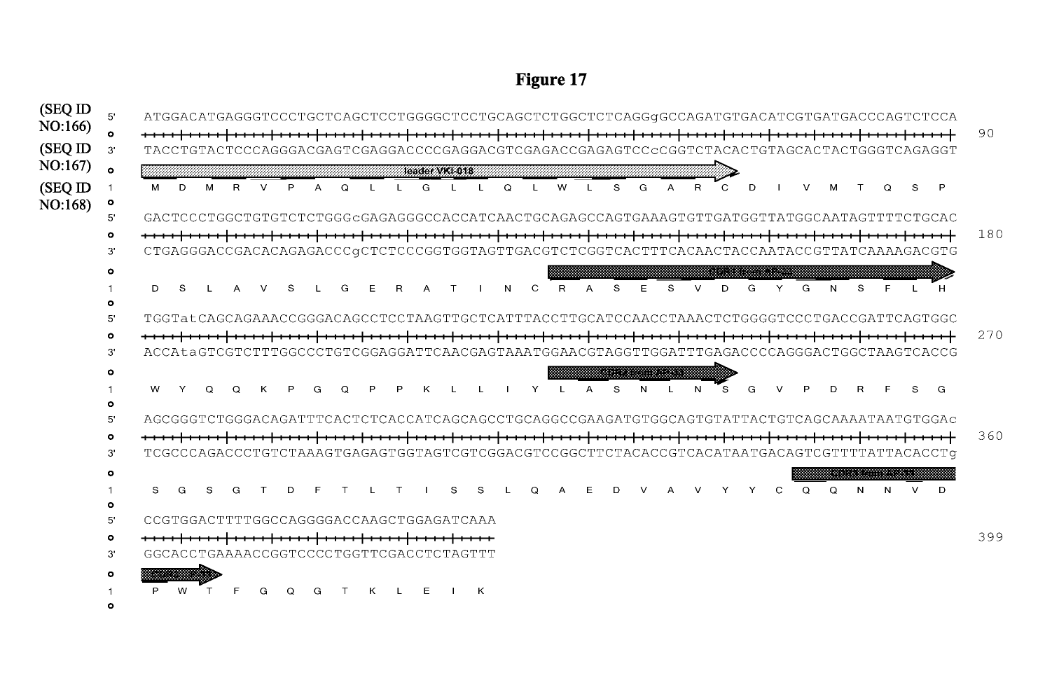

Two further light chain frameworks were tested based on the human sequences AB064133 and AB064072 that became humanized kappa light chains RK3 and RK4, respectively. The selection of AB064133 is shown in FIGS. 9-11 and 12. The VCI residues are defined in FIG. 13, while the protein and DNA sequences for both RK3 and RK4 are shown in FIGS. 6 and 14-17. RK3 was chosen because it was a from a different germline family, i.e. V kappa 5. Although AB064072 was a member of the Kabat VKIV subgroup, which have historically been poorly expressed when used in recombinant antibody constructs, this specific human framework sequence had been shown previously to express well in our hands when part of a humanized antibody.

### Example 1C

### Expression of Recombinant Heavy and Light Chains

Recombinant antibody V regions were expressed by transient transfection of Cos7 cells. Chimeric AP33 heavy or light chain DNA constructs were used as positive controls and co-transfected with the appropriate humanized antibody constructs chains. Initially the unmutated RHA and RHb-h (in which all seven unconserved vernier zone VC back-mutations) and RKAbd and RK2bc were tested. RK2bc has both conflicting VC residues back-mutated. The light chain RKA had two residues replaced; the VC residue Y36F, and since Asn is highly unusual at position 107, it was also replaced with Lys (RKAbd). It was noted that RKAbd expression was very low and below viable experimental and commercial levels (Table 3). In subsequent experiments modifications were made to RKAbd including removing potential splice sites mutation G380C and exchanging leader sequences from leader B3 to L11, shown in Table 4, which from our experience had worked efficiently in other light chain genes. In addition, others have reported that the amino acid substitution D9S was effective at rescuing expression of VKIV genes. See Saldanha J. W. et al. J Mol Biol Immunol 5391(22436):487709-99719 (1992). None of these modifications were effective in restoring expression levels above background.

TABLE 3
The leader sequences for RKA.

| | |
|---|---|
| B3 leader sequence | ATGGTGTTGCAGACCCAGGTCTTCATTTC |
| DNA | TCTGTTGCTCTGGATCTCTGGGGCTTACG |
| | GG (SEQ ID NO: 194) |
| B3 leader sequence | MVLQTQVFISLLLWISGAYG |
| Protein | (SEQ ID NO: 195) |
| L11 leader sequence | ATGGACATGAGGGTCCCCGCTCAGCTCCT |
| DNA | GGGGCTCCTGCTGCTCTGGCTCCCAGGCG |
| | CCAGATGT (SEQ ID NO: 196 |
| L11 leader sequence | MDMRVPAQLLGLLLLWLPGARC |
| Protein | (SEQ ID NO: 197) |

TABLE 4
Expression of humanized light chains. COS 7 cells were transfected by
the stated antibody constructs.

| | | Control transfection |
|---|---|---|
| | | chimeric antibody |
| Heavy Chain RHb-h | Antibody yield | (V_{H}/V_{L}) |
| RKAbd/VH | Not detected | 1008 ng/ml |
| RKAbd (leader plus | Not detected | 1415 ng/ml |
| D9S)/VH | | |
| RK2bc/VL | 389 ng/ml | 3335 ng/ml |
| RK2bc/RHb-h | 1555 ng/ml | 3335 ng/ml |
| RK2b/RHb-h | 1552 ng/ml | 849 ng/ml |
| RK2b/RH-C | 1222 ng/ml | 1036 ng/ml |
| RK3/VH | 39 ng/ml | 1518 ng/ml |
| RK3/RHb-h | 2.3 ng/ml | 1518 ng/ml |
| RK4/RHb-h | 124 ng/ml | 5161 ng/ml |

Moreover, alternative light chain constructs RK3 and RK4 also show extremely low levels of expression (Table 4). Only RK2 can be expressed at levels suitable for producing a humanized antibody.

### Example 1D

### The binding of RHb-h/RK2bc to E2 Peptides

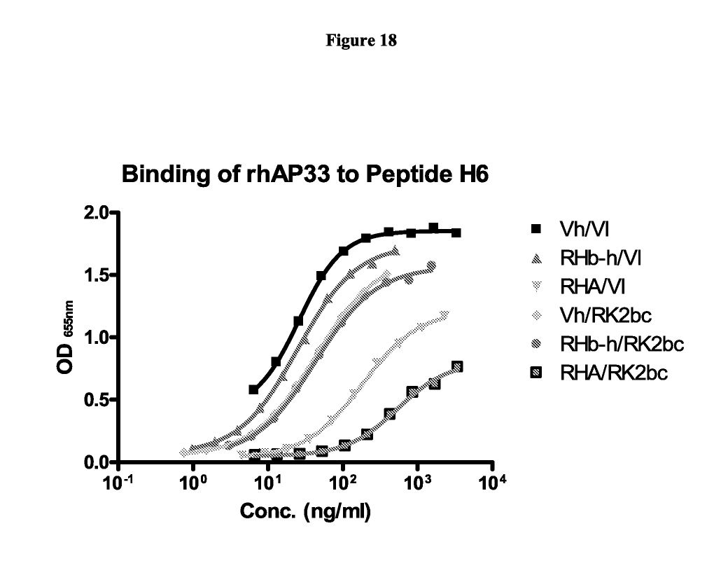

The supernatants from the Cos7 transfections were used to compare the binding of the humanized antibody RHb-h/RK2bc with the chimeric antibody. See FIG. 18.

The results from the antibody binding to the H6 mimotope suggest that the vernier zone (Foote J. and Winter G., J Mol Biol 224:487-99 (1992)) and canonical residues (Chothia C. et al., J Mol Biol 186:651-63 (1985), and Chothia C. et al., Nature 342:877-83 (1989)) introduced into RHA are necessary for binding. The H6 peptide binding of the antibody RHb-h/Vl was the closest to the chimeric antibody (Vh/Vl) positive control and better than the fully humanized RHb-h/RK2bc antibody suggesting that the humanized light chain is not as good as the chimeric light chain. This is emphasized by the particularly poor binding of RHA/RK2bc when it is compared to the chimeric light chain RHA/Vl.

The conclusion from this experiment is that the heavy chain RHb-h has retained much of the structural features in AP33 VH critical to antigen binding, but that the humanized light chain is less good. However, the absence of a human orthologue of the mouse light chain gene with the same L1 loop length was anticipated as a potential problem for humanization.

### Example 1B

### The Interface Between the Heavy and Light Chains Mediated by Humanized Heavy Chain Interface Residue Q39 was not Responsible for the Suboptimal Binding

One explanation for the poor function of the light chain was that the interface residues between the heavy and light chains were incompatible. See Chothia C. et al., J Mol Biol 186:651-63 (1985). The interface glutamine residues at position 39 was mutated back to the mouse equivalent, lysine, in RHb-h and the new heavy chain denoted as RHI.

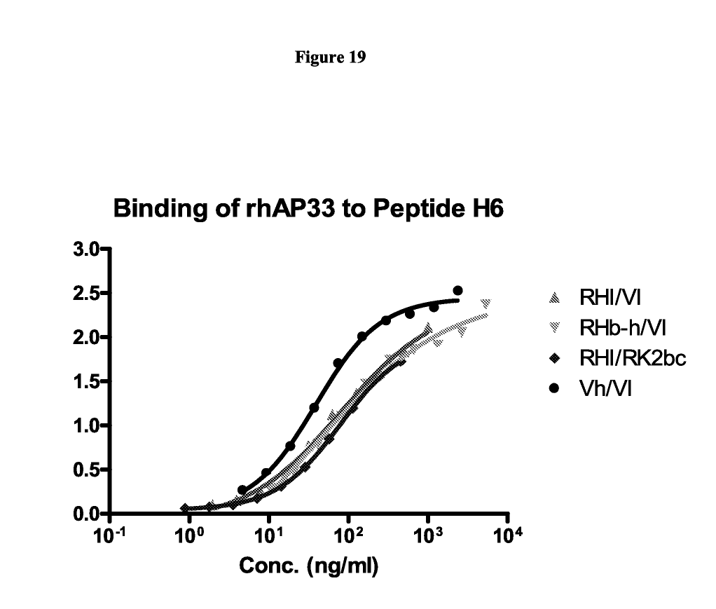

The binding of RHI paired with RK2bc or the chimeric light chain failed to improve binding to the H6 peptide suggesting that the interface residue Q39K was not responsible for the suboptimal binding shown in FIG. 19.

### Example 1E

### The Binding of RHb-h to a Range of E2 Peptides

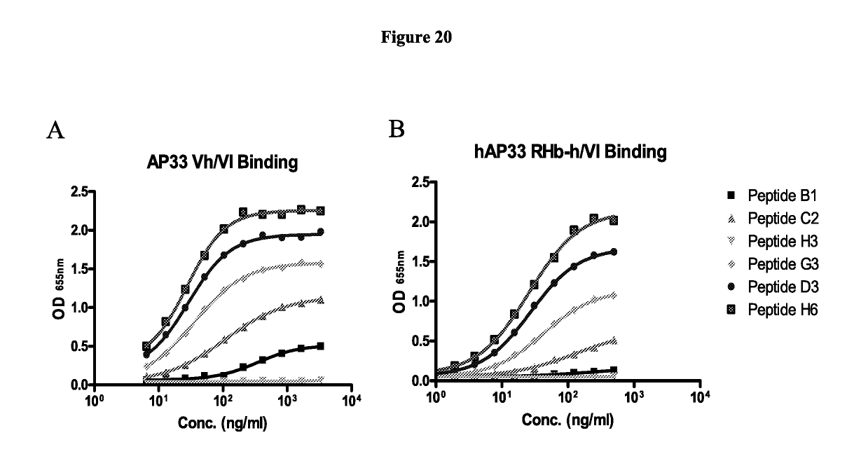

In order to estimate the binding of the humanized antibody to different HCV genotypes the peptides shown in Table 5 were used as proxies for a spectrum of E2 variants. The peptide binding of RHb-h paired with the chimeric light chain, V1, is shown in FIG. 20B. The results show that the humanized heavy chain binds a spectrum of peptides but is not as effective as the chimeric antibody, Vh/Vl, shown in FIG. 20A. Indeed the humanized antibody does not appear to bind peptide B1. The results indicated that replacing the unconserved canonical and vernier zone residues in the heavy chain was insufficient to retain the full spectrum of peptide antigen binding.

TABLE 5
Peptides used in the binding analysis of
humanized AP33.

| SEQ | | | |
|---|---|---|---|
| ID | | | |
| NO: | PEPTIDE | NAME | GENOTYPE |
| 198 | QLINTNGSWHINGSGK-biotin | D3 | All |
| 199 | N...........GSGK-biotin | B1 | 2b |
| 200 | ..........V.GSGK-biotin | C2 | 1a |
| 201 | ....S.......GSGK-biotin | H3 | 2a, 4 |
| 202 | ..V.........GSGK-biotin | G3 | 1a, 3 |
| 203 | VELRNLGGTWRPGSGK-biotin | H6 | Mimotope |

It is interesting to note that the peptides with variant AP33 epitopes have conserved changes, for example B1 replaces Gln to Asn and the peptides C2 and G3 are Ile to Val replacements. These conservative changes to smaller residues may represent a contraction of the epitope. This raises the possibility that the antigen contact residues on AP33 and the humanized antibody may be altered to give them greater reach. The effect of this could be to enhance the antibody's binding to those HCV genotypes which include the shorter epitope residues and that show weaker binding to AP33. It is also important to note that although the humanized antibody fails to bind the B1 peptide this sequence has not been found in infectious isolates of HCV.

### Example 1F

### Identifying the Minimal Mutations to RK2

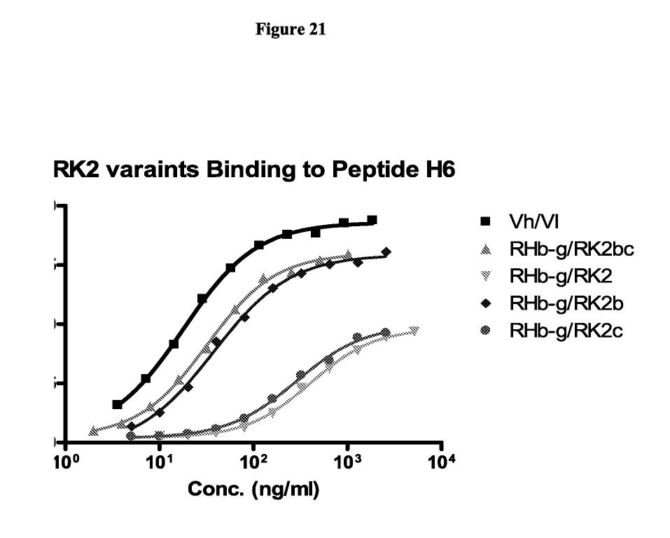

There were only 2 VC residues that were unconserved and were mutated in the RK2 light chain (mutation b (Y36F) and mutation c (G68R). Each VC mutation was back mutated to the human equivalent residue, Y36 and G68. The results (FIG. 21) show that mutation b is essential for light chain activity whereas mutation c is not.

### Example 1G

### Identifying the Minimal Number of VC Changes Necessary for the Heavy Chain Humanization

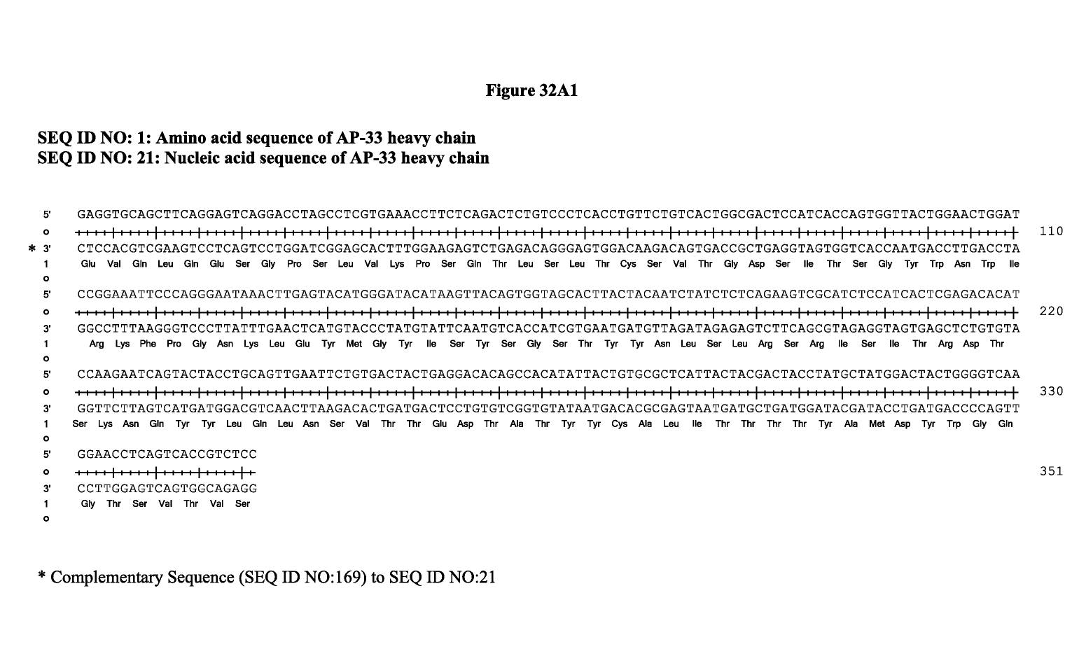

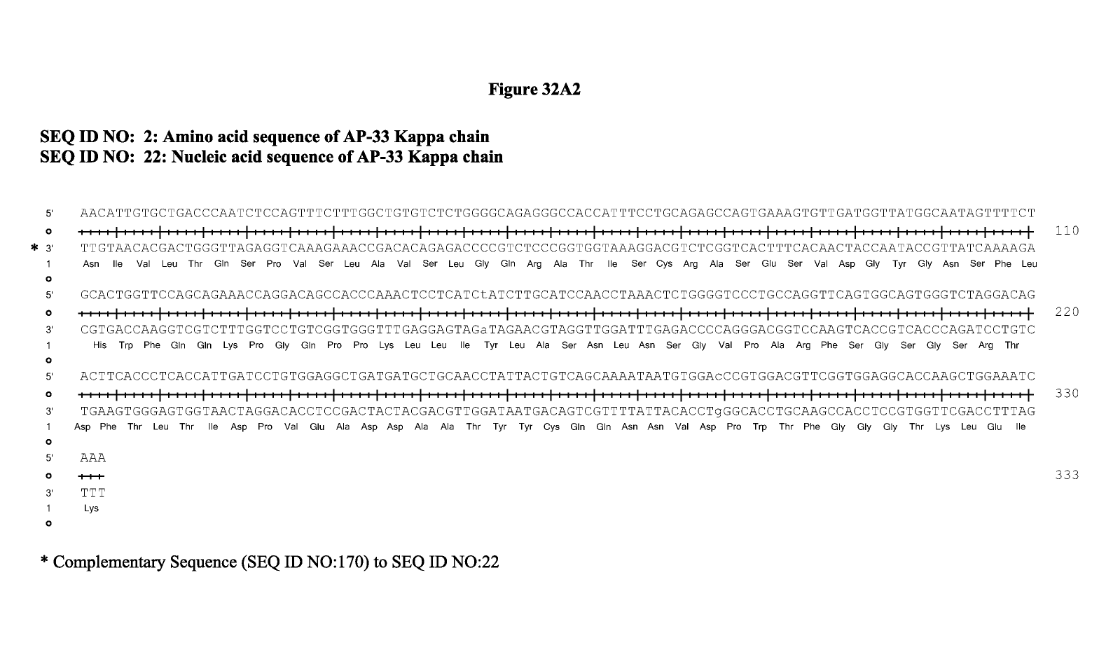

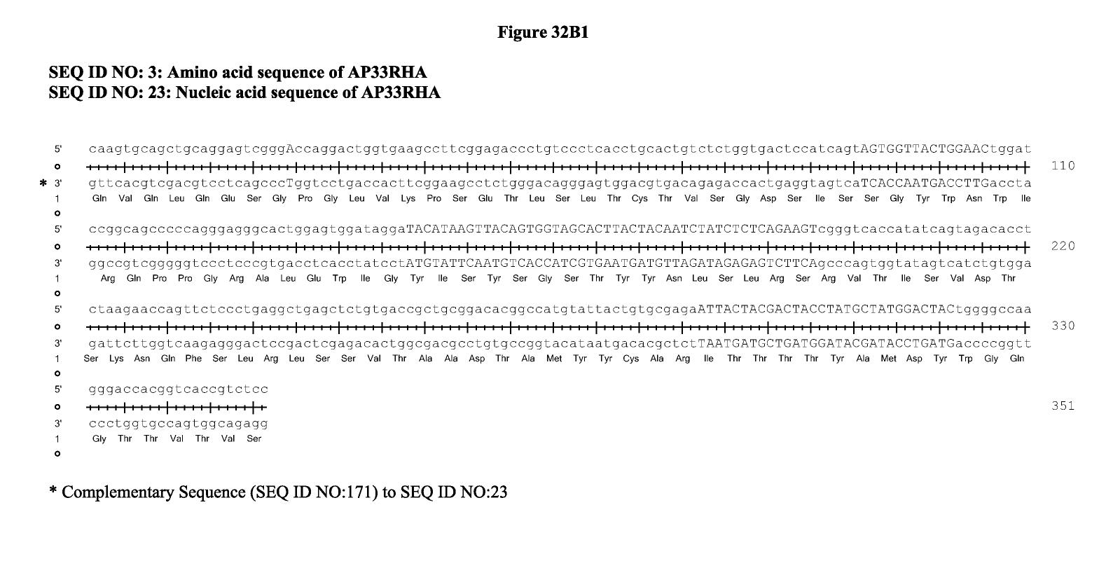

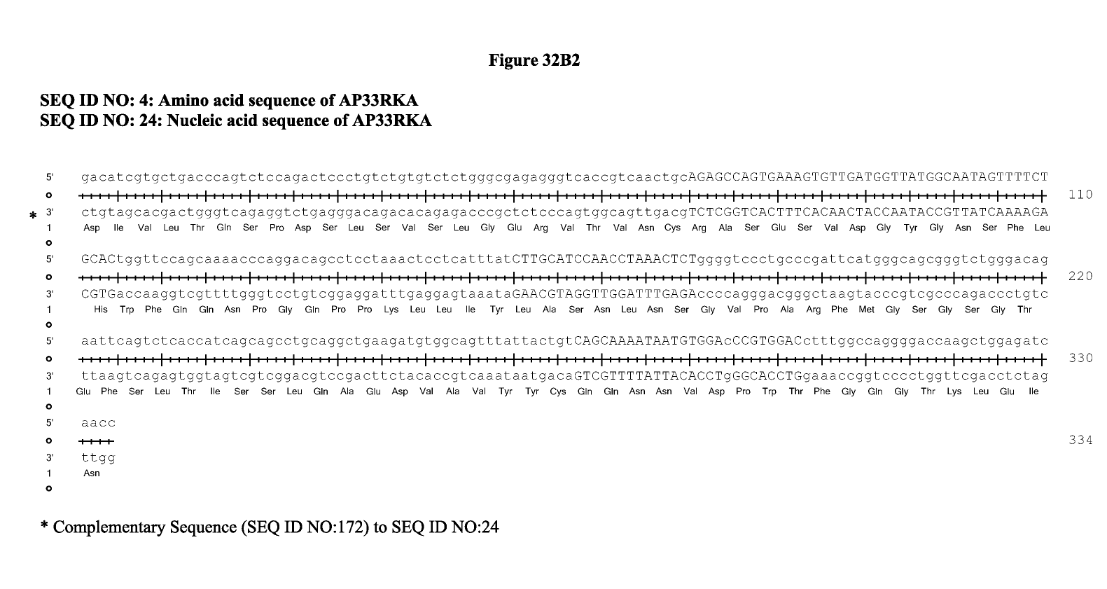

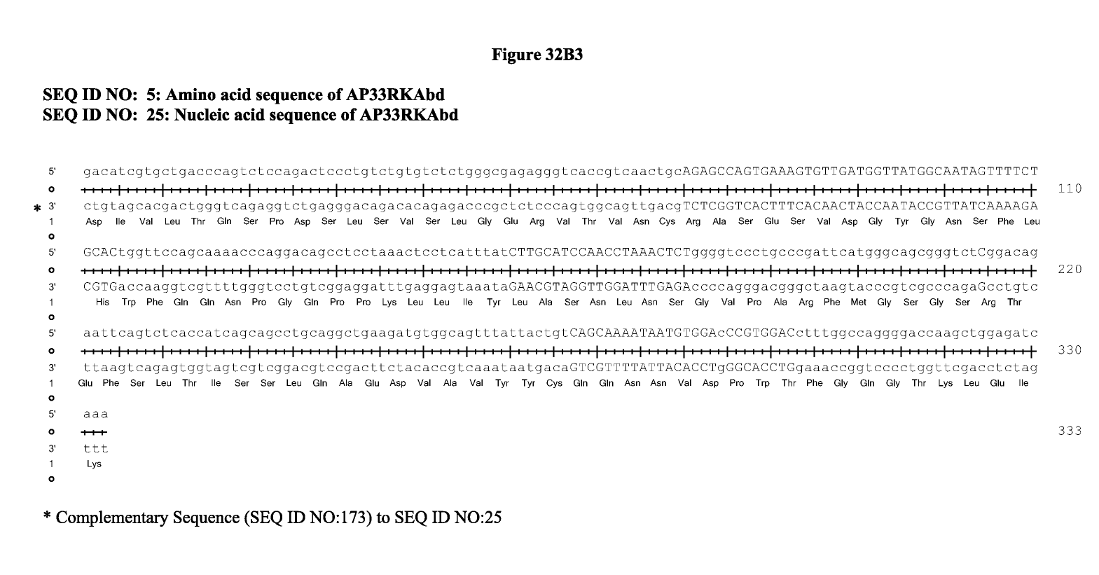

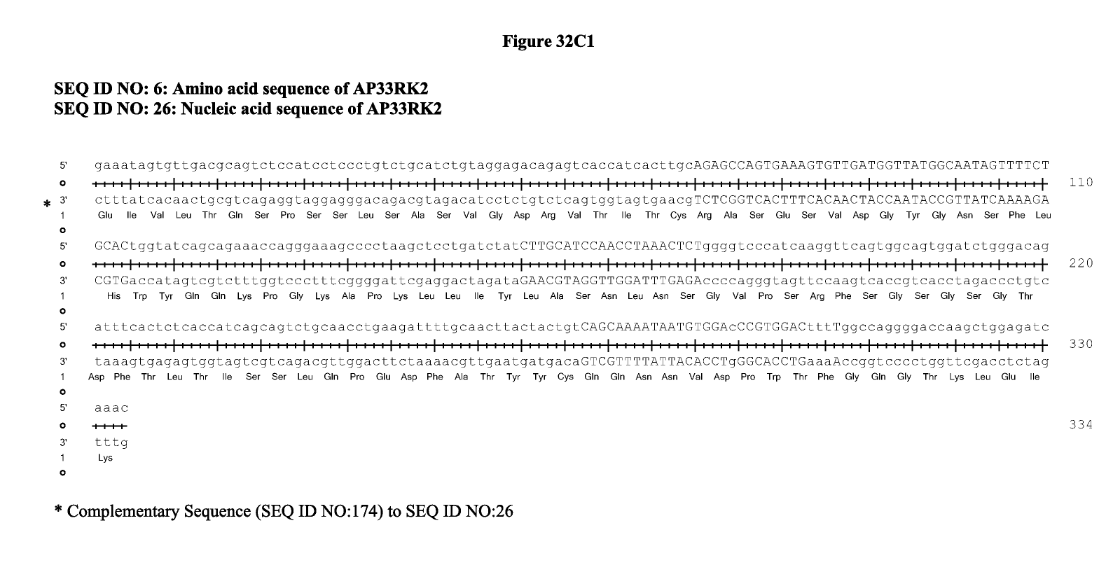

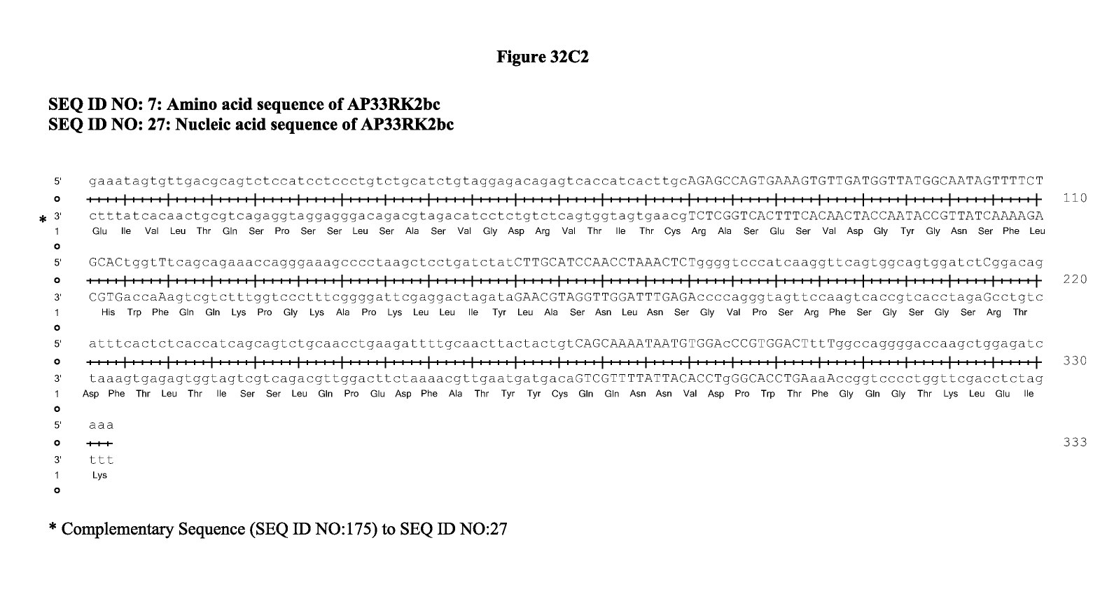

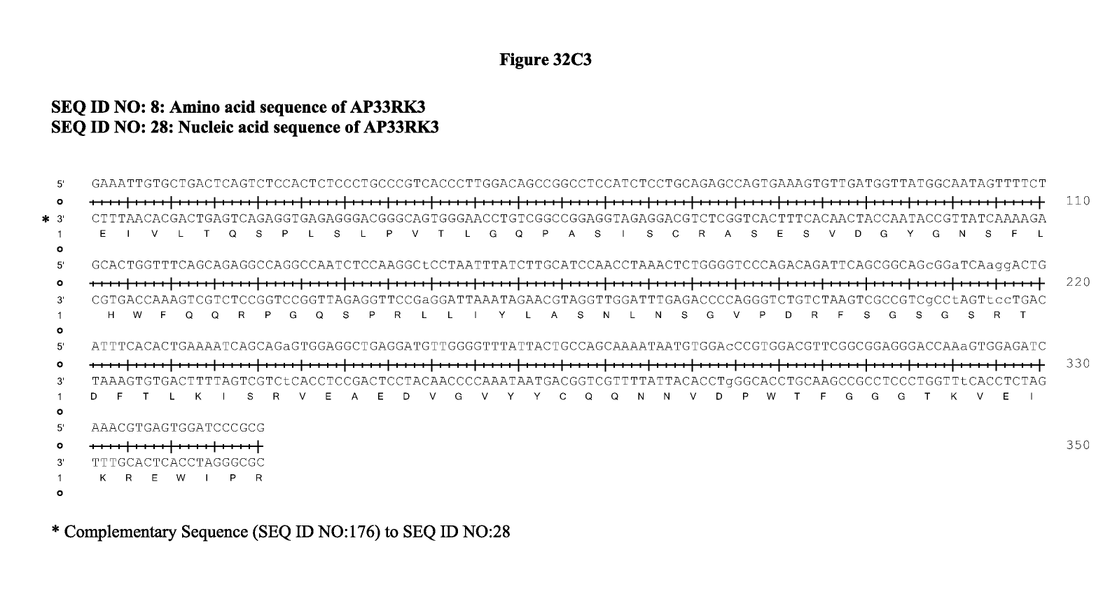

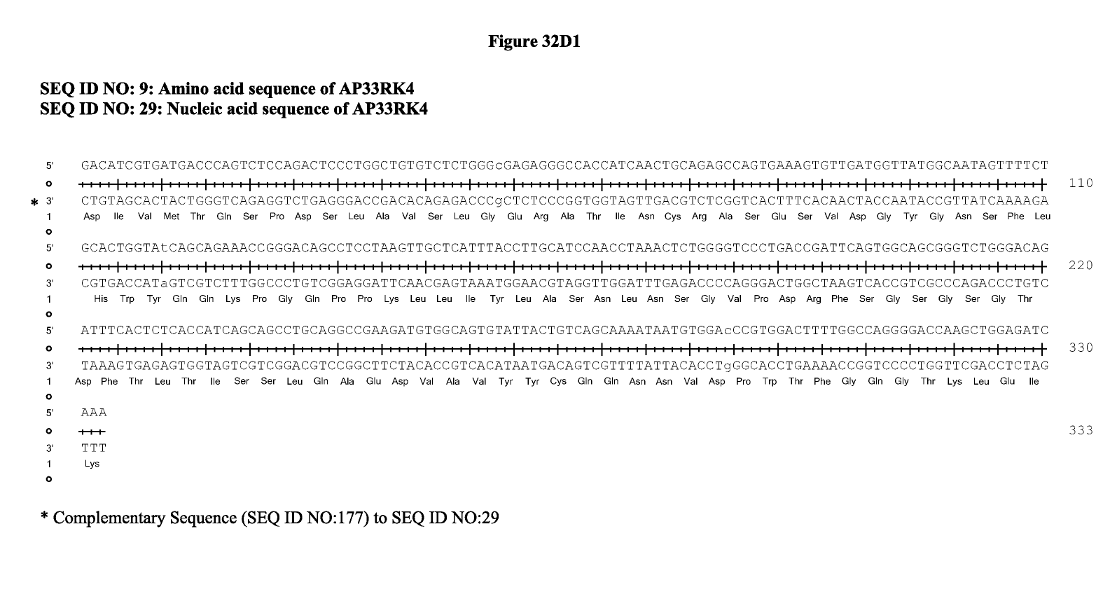

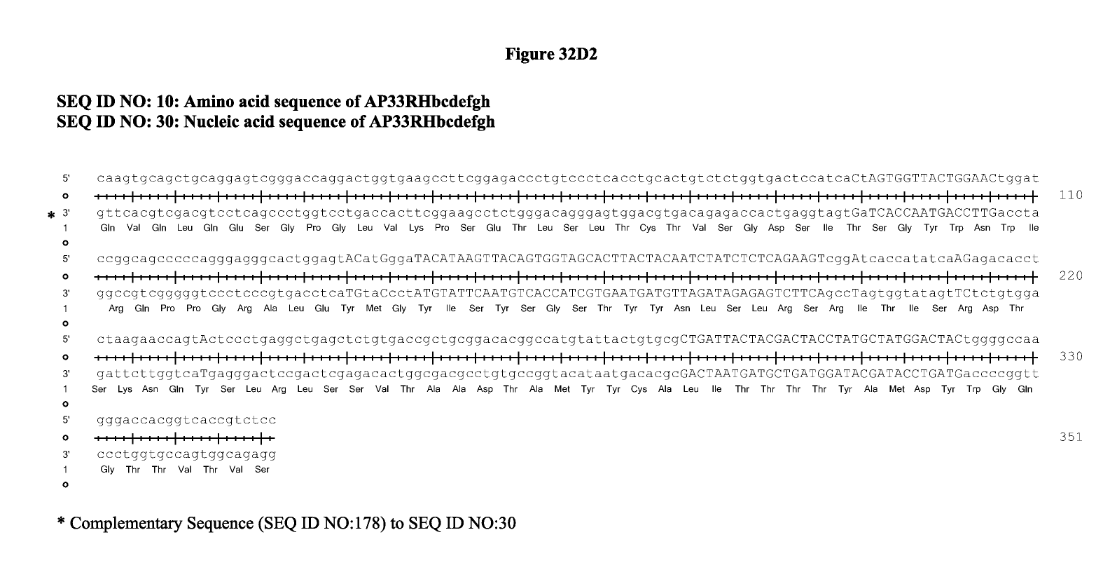

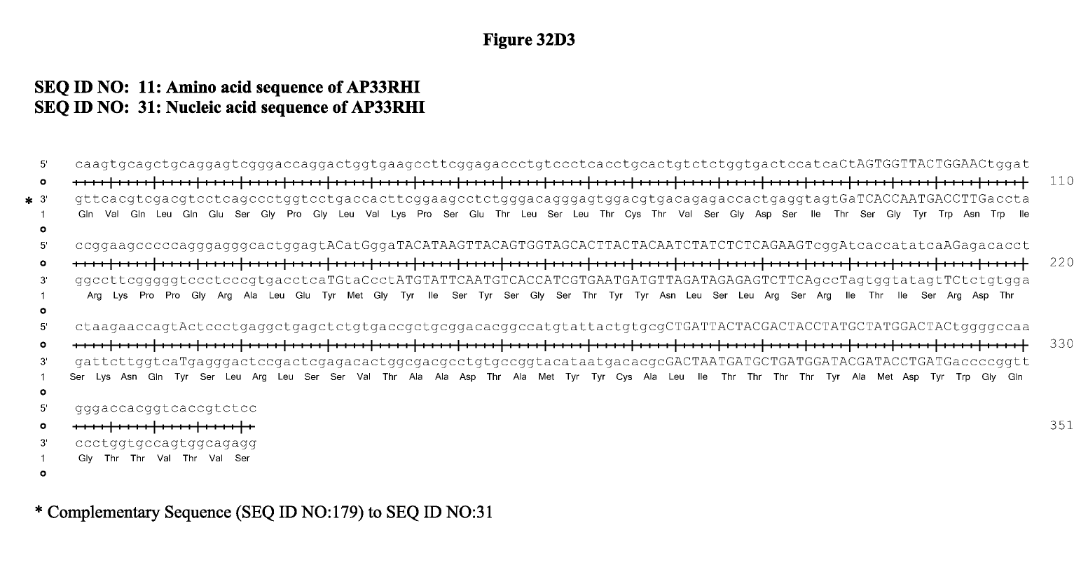

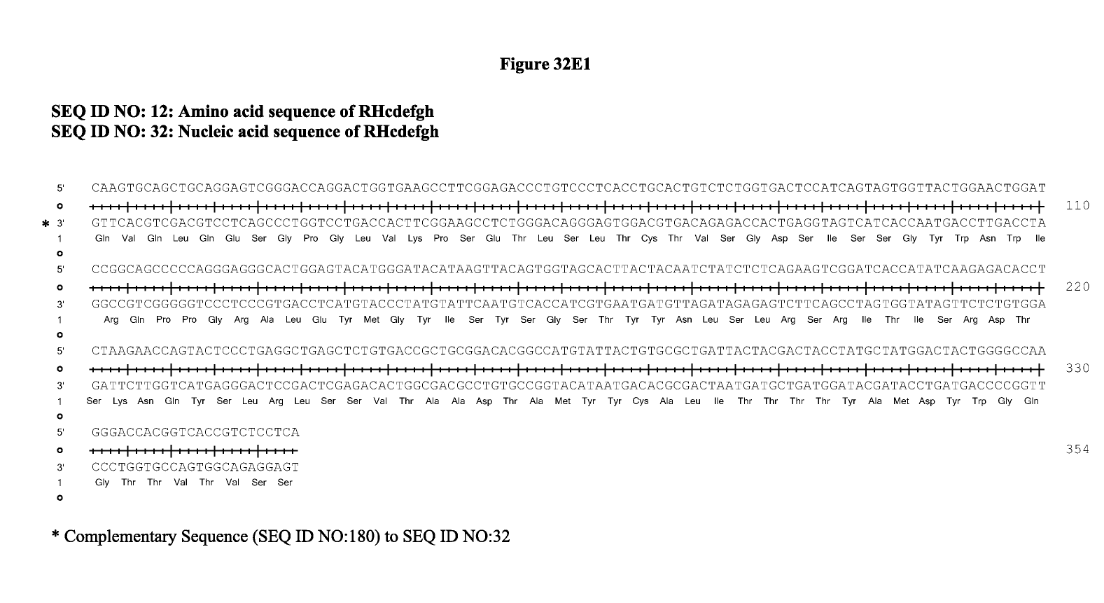

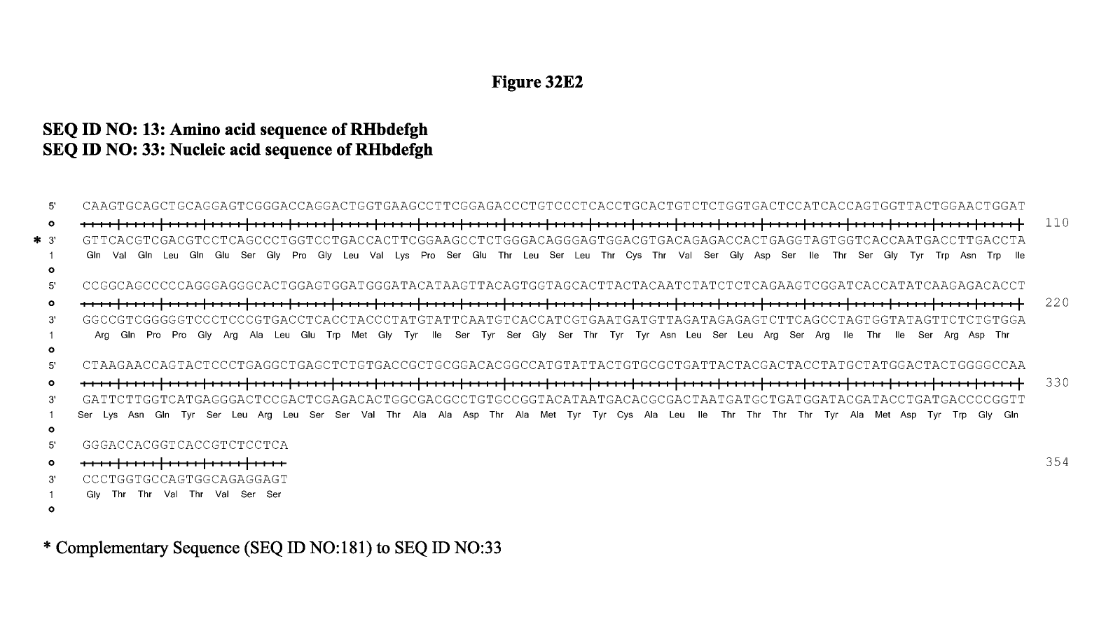

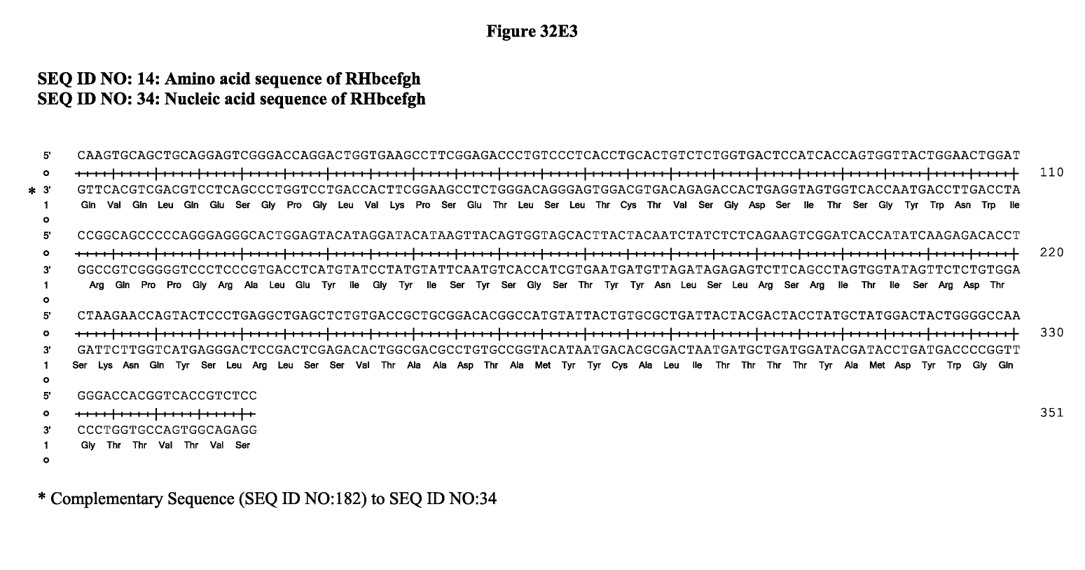

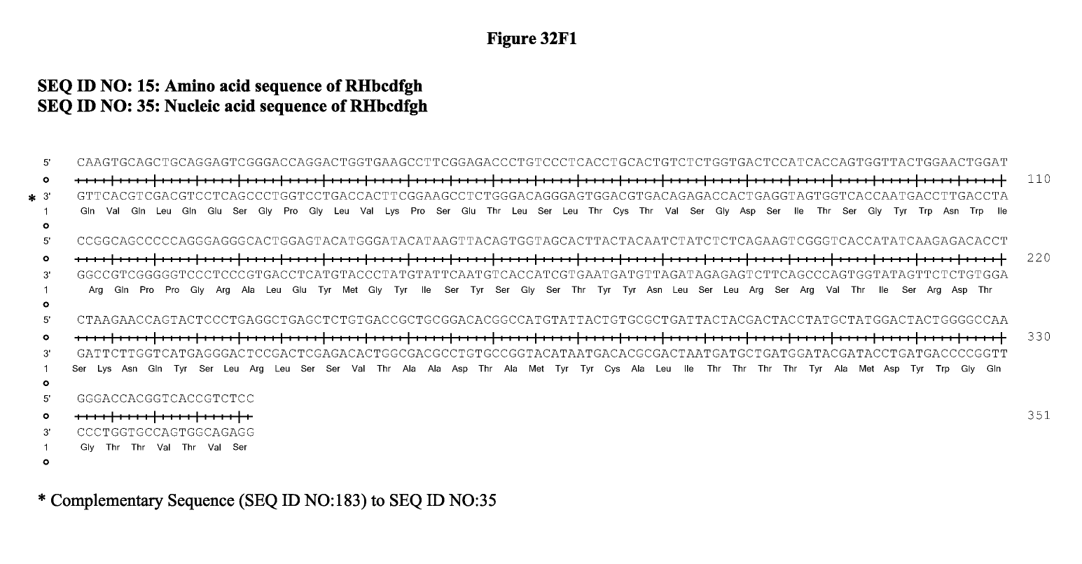

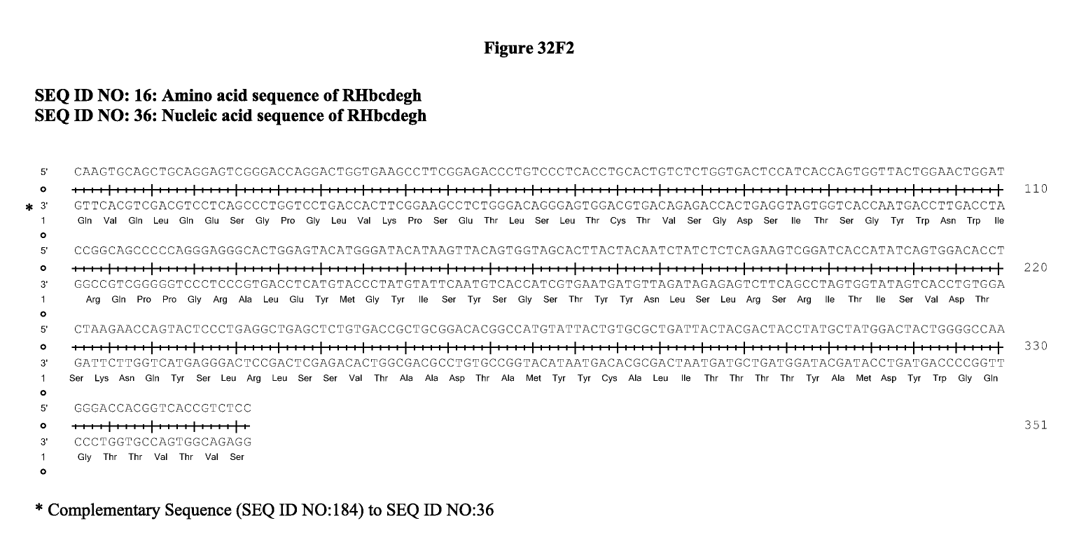

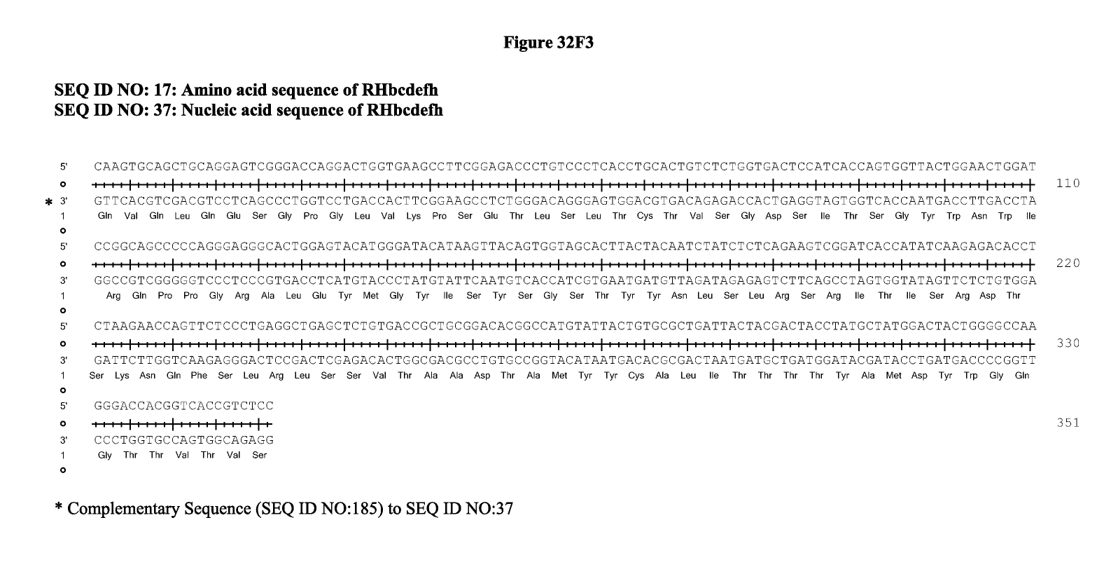

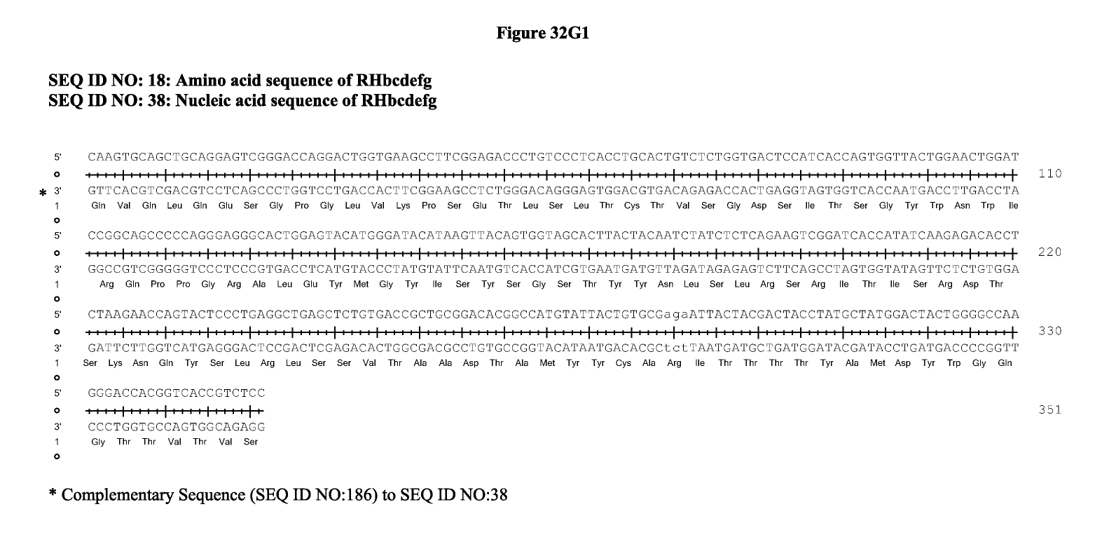

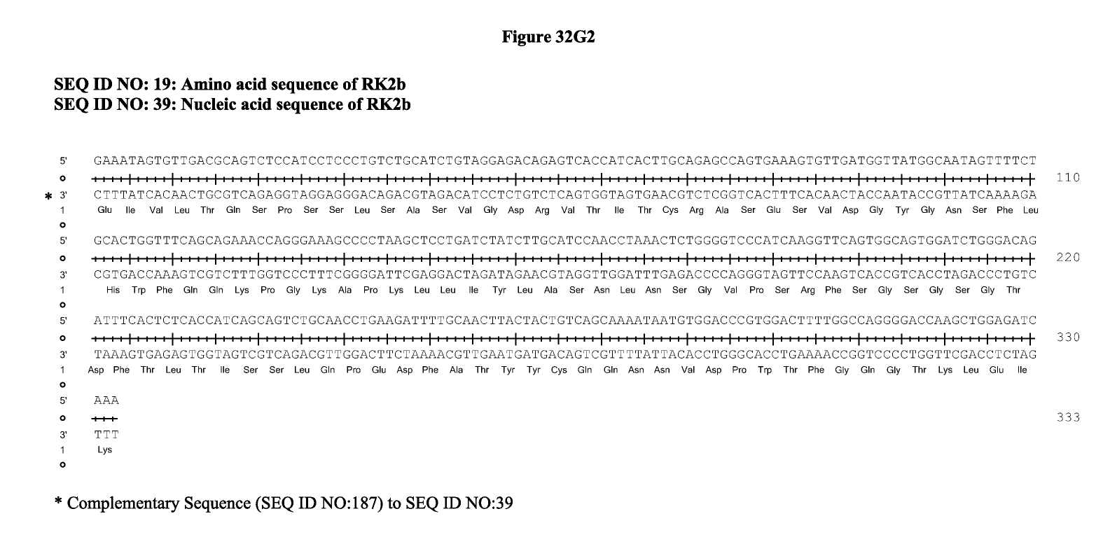

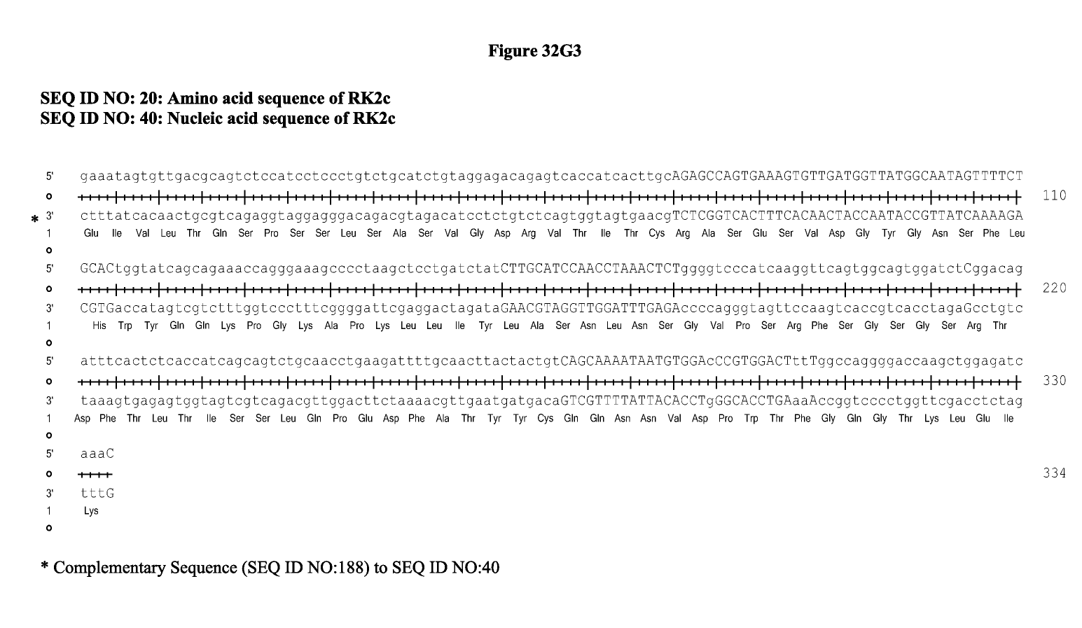

The effect of the mutations in Table 6 was assessed by comparing the binding to the peptides described in Table 5 of different versions of the humanized antibody. See also FIGS. 32A-G and U.S. Provisional Application 61/006,066, which is incorporated herein by reference in its entirety.

TABLE 6
Humanized antibody mutants and there sequence identification.

| | V chain | Mutations from | Amino Acid | Nucleic Acid |
|---|---|---|---|---|
| V gene name | name | parent sequence | SEQ ID NO | SEQ ID NO |
| AP33H heavy | VH | | SEQ ID NO: 1 | SEQ ID NO: 21 |
| chain | | | | |
| AP33H light | Vl | | SEQ ID NO: 2 | SEQ ID NO: 22 |
| chain | | | | |
| AP33RHA | RHA | None | SEQ ID NO: 3 | SEQ ID NO: 23 |
| AP33RKA | RKA | | SEQ ID NO: 4 | SEQ ID NO: 24 |
| AP33RKAbd | RKAbd | Y36F, N107K | SEQ ID NO: 5 | SEQ ID NO: 25 |
| AP33RK3 | RK3 | | SEQ ID NO: 8 | SEQ ID NO: 28 |
| AP33RK4 | RK4 | | SEQ ID NO: 9 | SEQ ID NO: 29 |
| RHbcdefgh | RHb-h | S30T, W47Y, I48M, | SEQ ID NO: 10 | SEQ ID NO: 30 |
| | | V67I, V71R, F78Y, | | |
| | | R94L | | |
| RHcdefgh | RH-B | W47Y, I48M, V67I, | SEQ ID NO: 12 | SEQ ID NO: 32 |
| | | V71R, F78Y, R94L | | |
| RHcdefgh | RH-C | S30T, I48M, V67I, | SEQ ID NO: 13 | SEQ ID NO: 33 |
| | | V71R, F78Y, R94L | | |
| RHcdefgh | RH-D | S30T, W47Y, | SEQ ID NO: 14 | SEQ ID NO: 34 |
| | | V67I, V71R, F78Y, | | |
| | | R94L | | |
| RHcdefgh | RH-E | S30T, W47Y, I48M, | SEQ ID NO: 15 | SEQ ID NO: 35 |
| | | V71R, F78Y, R94L | | |
| RHcdefgh | RH-F | S30T, W47Y, I48M, | SEQ ID NO: 16 | SEQ ID NO: 36 |
| | | V67I, F78Y, R94L | | |
| RHcdefgh | RH-G | S30T, W47Y, I48M, | SEQ ID NO: 17 | SEQ ID NO: 37 |
| | | V67I, V71R, R94L | | |
| RHcdefgh | RH-H | S30T, W47Y, I48M, | SEQ ID NO: 18 | SEQ ID NO: 38 |
| | | V67I, V71R, F78Y | | |
| RHI | RHI | Q39K | SEQ ID NO: 11 | SEQ ID NO: 31 |
| RK2 | RK2 | none | SEQ ID NO: 6 | SEQ ID NO: 26 |
| RK2b | RK2b | Y36F | SEQ ID NO: 19 | SEQ ID NO: 39 |
| RK2c | RK2c | G68R | SEQ ID NO: 20 | SEQ ID NO: 40 |
| RK2bc | RK2bc | Y36F G68R | SEQ ID NO: 7 | SEQ ID NO: 27 |

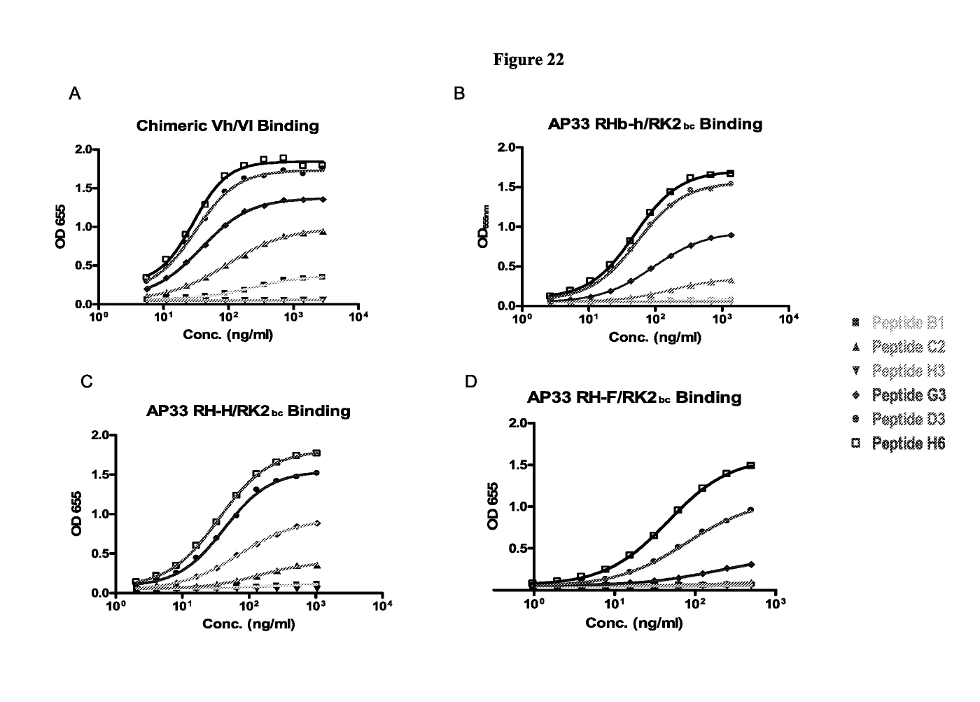

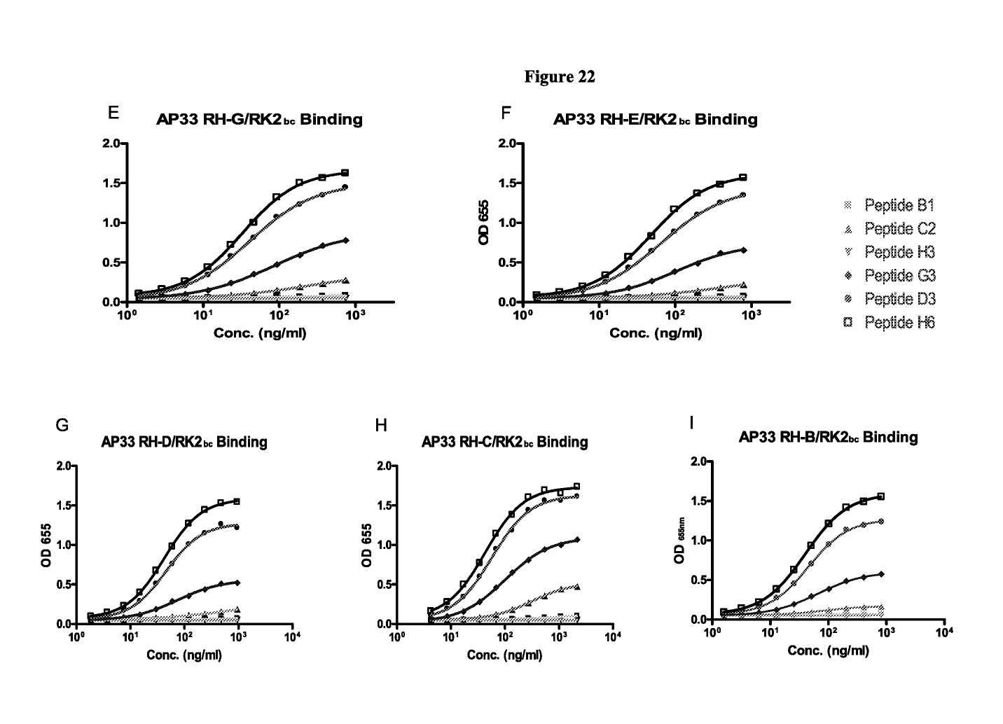

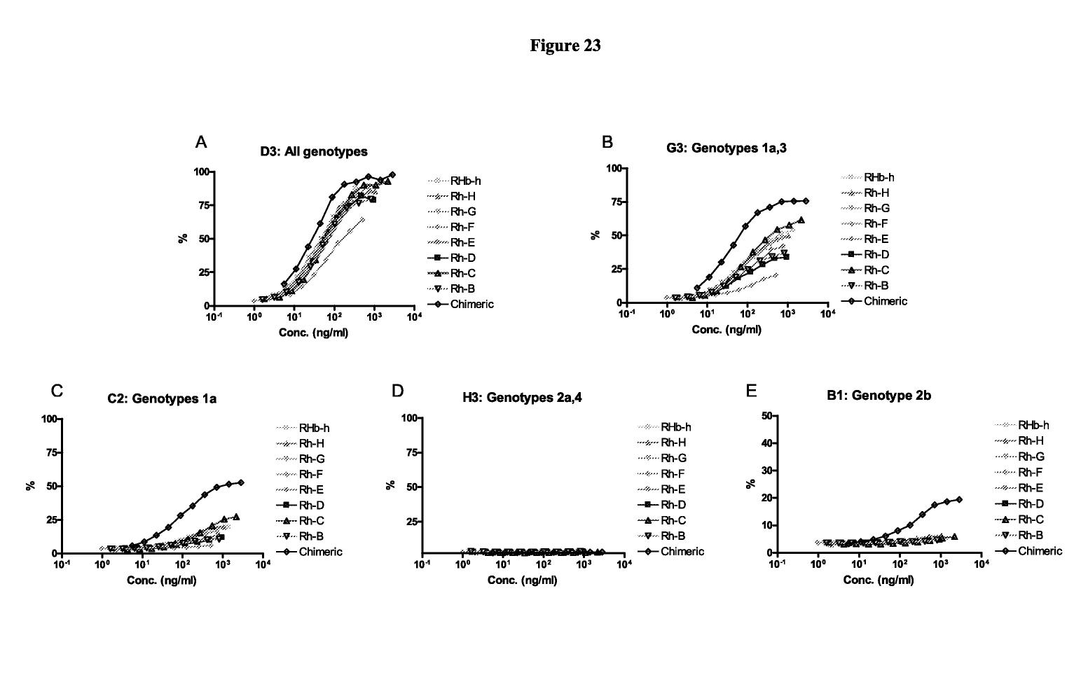

The results are shown in FIG. 22 and normalized in FIG. 23 by expressing each data set as a percentage of the maximum binding to H6. The results from antibody RH-F suggested that the absence of the VC mutation F (V71R) significantly affected the binding of the antibody. Therefore, despite the presence of all other VC mutations from human to the mouse sequence, Arginine at position 71 is necessary for optimal binding. In all cases where binding could be detected, RH-F bound to the peptides more weakly. However, binding to peptide G3 by the back-mutated variants also identified the original mutations S30T (b), 148M (d) and V67I (e) as being important for binding affinity. Mutations F78Y (g) and R94L (h) were essentially indistinguishable (displaying only marginally less binding when back-mutated, when compared to the RHb-h standard) and so did not appear to be critical to peptide binding. However, antibody version RH-C resulted in an increased binding to peptides G3 and C2 over all other variants, including RHb-h (FIG. 23). In this case mutation c (W47Y) is not present and the human tryptophan residue is retained (but VC mutations S30T, I48M, V67I, V71R, F78Y, and R94L are present). On this basis the humanized antibody containing the heavy chain variant RH-C was chosen to be tested in the HCVpp assays and compared to RHb-h. The humanized antibody RH-H (where the mutation h (R94L) is not present) was also included for testing in the HCVpp assays. The R94L mutation is a canonical and vernier zone residue that supports the H3 loop and we wished to determine if disruption of the H3 loop adversely affected inhibition of HCVpp infection. These heavy chains were co-expressed with the humanized light chain variant RK2b.

### Example 1H

### HCV Pseudoparticle Infection Assays

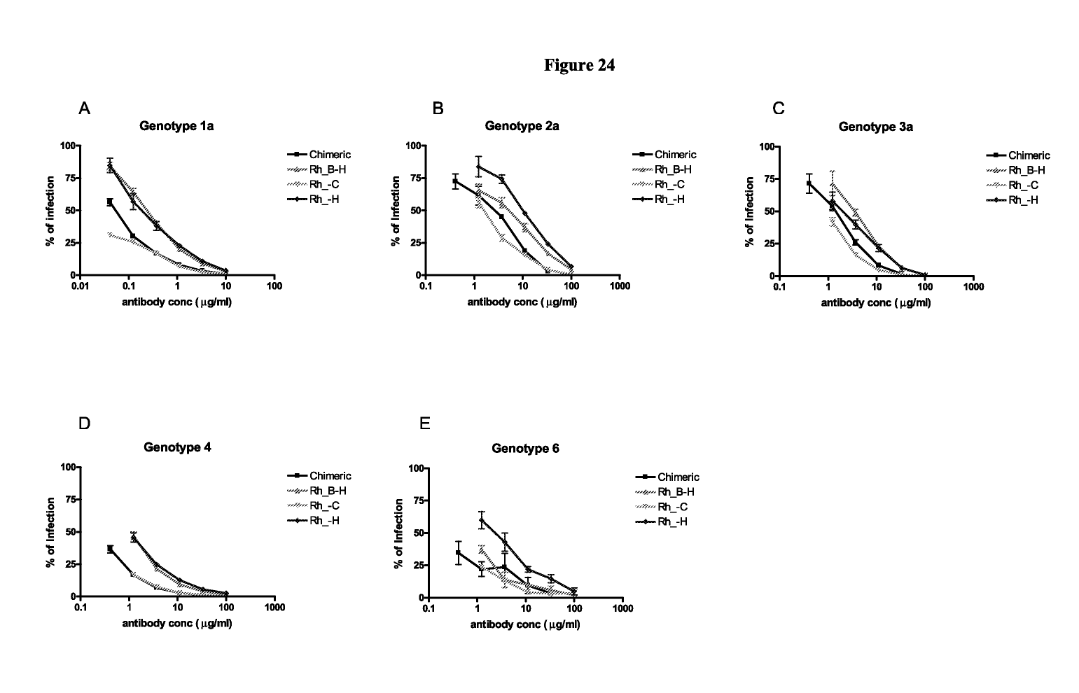

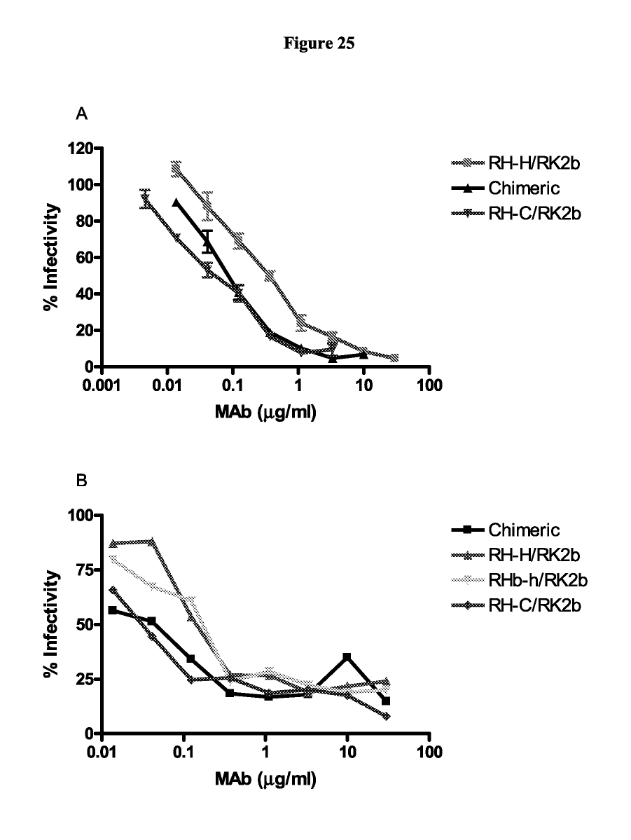

Three humanized antibodies were tested in the HCVpp infection assays. All humanized heavy chains were paired with light chain RK2b. Although the peptide binding data suggested little difference between the heavy chains RHb-h, RH-C and RH-H, the data shown in FIGS. 24 and 25 suggested that RH-C is the best inhibitor of HCVpp infection. The RH-C antibody was at least as effective as the positive control chimeric AP33, at inhibiting HCVpp infection but the other humanized antibodies RHb-h and RH-H were significantly less effective. The IC50 and IC90 for four experiments are shown in Table 2 and show that the humanized antibody shows very similar IC50 and IC90 values to that of the chimeric antibody across all genotypes.

This result was unexpected since the location of the back-mutation in RH-C (i.e., residue position 47), is both a vernier and interface residue suggesting that the tryptophan residue found in the original human FW may either improve the interface between the heavy and light chains, or may better support the H2 loop, or may do both.

The tryptophan residue present in the AP33 epitope has been shown to be crucial for AP33 binding. See Tarr A. W. et al., Hepatology 43:592-601 (2006). The Y47 residue lies directly underneath a lipophilic region of the CDRs and it is a reasonable supposition that the Y47W mutation helps to fill a gap at the base of the lipophilic region.

One method that may help to elucidate the nature of the improved binding mediated by the Y47W mutation is a kinetic analysis of antibody E2 binding. However, we have been unable to perform kinetic analysis of the interaction between RH-C and the E2 protein. The HCV E2 protein forms aggregates when purified. Unfortunately, monomeric E2 protein, which so far is unavailable, is necessary to measure binding affinity to antibody.

The data from the peptide analysis suggested that there is very little difference between the binding of heavy chain versions RH-G and RH-H. It would be interesting to test these versions combined with RH-C in the HCV pseudoparticle experiments. It is plausible that there may be a positive effect on binding and inhibition since these residues might help support the H2 and H3 loops respectively.

### Example 1I

### Analysis of the Chimeric Mutants AP33 Y47F and Y47W

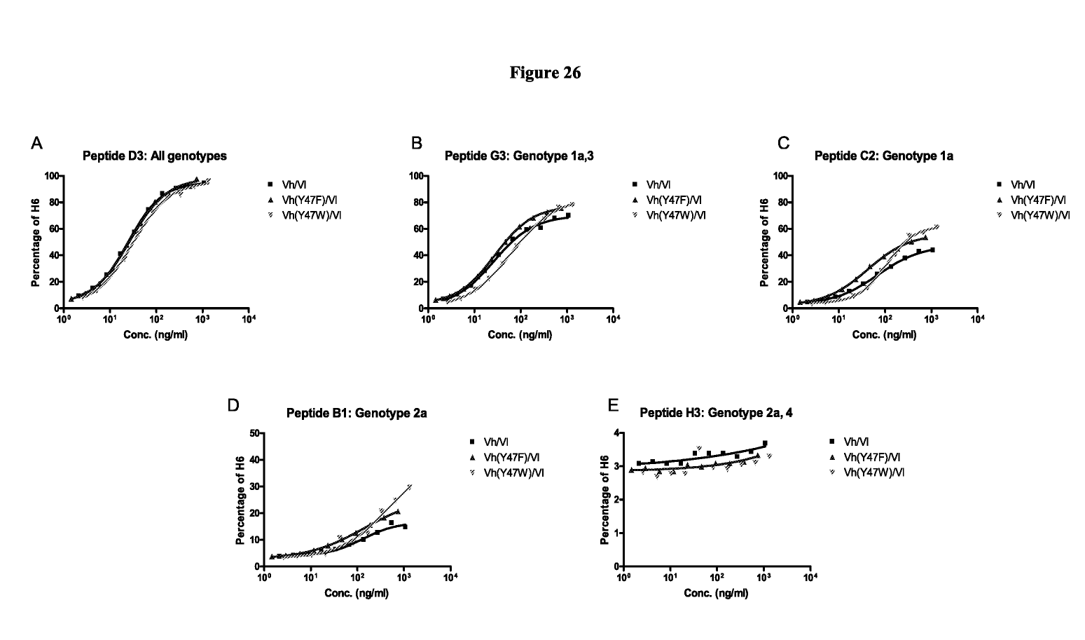

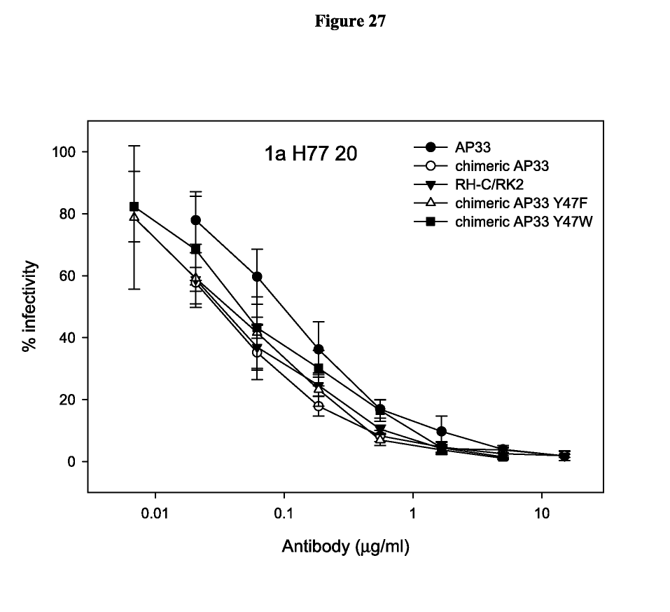

In order to further investigate the contribution of residue Y47 to binding, two chimeric heavy chain mutants were made, Y47W and Y47F. Both these mutants were expressed in association with the chimeric light chain, V1 and compared to AP33 in the HCVpp infection assays and peptide binding. The data from the peptide binding experiments (FIG. 26) suggest that making residue tyrosine 47 more hydrophobic, by substitution with a phenylalanine or tryptophan, may improve binding to some E2 peptides, especially peptide B1 (genotype 2a). However when the antibodies were used in the HCVpp infection assay against a genotype 1a (from isolate 1a H77.20) shown in FIG. 27, there was no enhancement of inhibition by either Y47W or Y47F mutation. It may be concluded therefore that the improved Y47W mutation in RH-C is specific to the humanization although further HCVpp infection assays need to be carried out on a variety of genotypes to determine if the Y47W mutation in AP33 may generally improve the antibody potency of infection inhibition.

### Example 2

### Materials and Methods

The following materials and methods were used for the experiments described in Example 2A-C.

Generation of Baculovirus Expressed Soluble E2 (sE2)

sE2 expression: Soluble E2 (sE2) were generated by deleting the transmembrane domains by truncating at amino acid 661 (sE2661) as described previously. See Roccasecca, R. et al., J Virol 77:1856-67 (2003). sE2661 was cloned into a baculovirus transfer vector co-transfected with BacPak6 linearized viral DNA (BD Clontech) into adherent Sf-9 insect cells cultured in ESF921 protein-free medium (Expression Systems, LLC) at 27° C. The resulting viral stock was amplified twice using standard baculovirus methods before use in large-scale protein production. The production was done in Wave™ bioreactors (GE Bioscience). Ten-liter T.ni Pro cells (Expression Systems, LLC) cultures were grown to 2×10⁶ cells/mL and infected with 50 mL of the viral stock as prepared above. The supernatant was harvested 48 hours post infection by centrifugation 3000×g for 15 minutes and filtered through a 0.2 μM filter prior to purification.

sE2 purification: The 10 L baculovirus supernatant was batched with 50 mL of Nickel-NTA resin. The HIS-tagged soluble E2 was eluted off of the resin with 250 mM Imidazole in PBS+0.3M NaCl. The elution was diluted into 20 mM NaAcetate, pH 5.0 and loaded over a 34 mL SpFF cation exchange column, and the protein was eluted off in the acetate buffer with 0.3M NaCl. The elution was then loaded over a 24 mL 5200 gel filtration column in PBS+0.15M NaCl and dialyzed into PBS buffer. In Source Decay using mass spectrometry, the N-terminus matched the expected N-terminus of the secreted protein.

Determining AP33/RH-C/RK2b Affinity to HCV E2

BIAcore assay: Surface plasmon resonance (SPR) measurements on a BIAcore A100 instrument were used to determine affinity for binding of soluble E2 (sE2) to antibody. A format of capture of the humanized antibody on an anti-human Fc sensor chip surface, followed by injection of a varied concentration of sE2, was employed. The anti-human Fc antibody was covalently linked to the sensor chip surface using amine chemistry, as suggested by the manufacturer. The humanized antibody was captured by injecting 60 μL of a 0.5 μg/mL solution at a flow rate of 30 μL/min. Sensorgrams were collected for 60 μL injections of sE2 solutions followed by monitoring of dissociation for 480 s. The sensor chip surface was regenerated by injection of a 15 μL aliquot of 3 M MgCl2 resulting in dissociation of the antibody-antigen complex from capture antibody. Measurements were repeated with sE2 concentrations ranging from 1.56 nM to 50 nM in 2-fold increments. All measurements included real-time subtraction of data from a reference flow cell with no captured anti-E2 antibody. A sensorgram for injection of buffer alone was also subtracted. The running buffer was Hepes-buffered saline, pH 7.2, and the temperature was 25° C. These data were analyzed with a 1:1 Langmuir binding model, using software supplied by the manufacturer, to determine the kinetics constants.

Scatchard analysis: Affinity of RH-C/RK2b to HCV E2, as part of the E1E2 heterodimer expressed on the surface of 293T cells, was determined using a radioligand cell binding assay. The anti-HCV antibodies, RH-C/RK2b and RH-C/RK2b Fab, were iodinated using the Iodogen method. The radiolabeled anti-HCV antibodies were purified from free 1251-Na by gel filtration using a NAP-5 column. The purified RH-C/RK2b and RH-C/RK2b Fab antibodies had a specific activity of 17.96 μCi/μg and 55.21 μCi/μg, respectively. Competition reaction mixtures of 50 μL volume containing a fixed concentration of iodinated antibody and decreasing concentrations of serially diluted unlabeled antibody were placed into 96-well plates. 293T cells were transfected with Fugene6 transfection reagent (Roche) as per manufacturer's recommendations. Cells were transfected with 25 μg/mL plasmids plus 100 μL Fugene6 reagent in a final volume of 25 mL of Freestyle medium (Invitrogen, Gibco) without any supplements. Cells were detached from plates 48 hours post transfection using Sigma Cell Dissociation buffer, washed with binding buffer (50:50 DMEM/F12 with 2% FBS, 50 mM HEPES, pH 7.2, and 2 mM sodium azide) and added at an approximate density of 2×10⁵ cells in 0.2 mL of binding buffer to the 50 μL competition reaction mixtures. The final concentration of the iodinated antibody in each competition reaction with cells was ˜200 μM for RH-C/RK2b and ˜500 μM for RH-C/RK2b Fab and the final concentration of the unlabeled antibody in the competition reaction with cells varied, starting at 500 nM and then decreasing by 1:2 fold for 10 concentrations. Competition reactions with cells were incubated at RT for 2 hours. Competition reaction with cells for each concentration of unlabeled antibody was assayed in triplicate. After the incubation, the competition reactions were transferred to a Millipore Multiscreen filter plate and washed 4× with binding buffer to separate the free from bound iodinated antibody. The filters were counted on a Wallac Wizard 1470 gamma counter (PerkinElmer Life and Analytical Sciences Inc.). The binding data was evaluated using NewLigand software (Genentech), which uses the fitting algorithm of Munson and Robard (Munson, P. J., and D. Rodbard, Anal Biochem 107:220-39 (1980)) to determine the binding affinity of the antibody.

Generation of Infectious Cell Culture HCV (HCVcc)

Generation of plasmids encoding full length HCVcc genomes: Full length HCV genomes for Jc1 (J6/C3) and Con1/C3 were chemically synthesized by outsourcing to Gene Oracle Inc. (Mountain View, Calif.) using DNA sequences for HC-J6(CH) (J6), JFH-1 and Con1 as described in the NCBI database [accession numbers AF177036, AJ238799 and AB047639 for HC-J6(CH) (clone pJ6CF), Con1 and JFH-1, respectively]. Chimeric HCVcc viruses that encode the J6 and Con1 structural regions (core-E1-E2-p7-part of NS2) fused to the JFH-1 NS2-NS5B region were generated as described previously (Pietschmann, T. et al., Proc Nail Acad Sci USA 103:7408-13 (2006)). To make the Con1/C3-neo HCVcc, a DNA fragment containing the 5′-untranslated region (UTR) followed by the neomycin resistance gene and the Encephalomyocarditis virus internal ribosome entry site (EMCV IRES) element flanked by EcoRI and PmeI restriction sites was chemically synthesized by outsourcing to Gene Oracle Inc. (Mountain View, Calif.). The plasmid encoding Con1/C3-neo HCVcc was generated by digesting with EcoRI and PmeI. Both Jc1 (J6/C3) and Con1/C3-neo DNA fragments were ligated into pUC19 vector using unique EcoRI and XbaI restriction sites to generate pUC-Jc1 and pUC-Con1/C3-neo.

In vitro transcription reactions: pUC-Jc1 and pUC-Con1/C3-neo plasmids were digested with XbaI, which is located at the 3′ end of the HCV genome. 30 μg of pUC-Jc1 and pUC-Con1/C3-neo were digested overnight at 37° C. using 20 U XbaI in a final volume of 300 The following day, RNA was extracted using acid phenol as described previously. See Kapadia, S. B. et al. J Virol 81:374-83 (2007). In vitro transcription reactions were performed using the T7 Megascript kit (Ambion) as per manufacturer's recommendations. HCV RNA was extracted using phenol/chloroform and ethanol precipitation, as described previously. See Kapadia, S. B. et al., J Virol 81:374-83 (2007)). RNA was stored at −70° C.

Generation of HCVcc stocks: Huh-7.5 cells were cultured in complete Dulbecco's modified Eagle's medium (c-DMEM) (supplemented with 10% fetal bovine serum [FBS], 100 U/ml penicillin, 100 mg/ml streptomycin, 2 mM L-glutamine, and 0.1 mM nonessential amino acids) under an atmosphere of 5% CO2 at 37° C. On the day of transfection, cells were trypsinized, washed twice with Opti-MEM medium (Gibco) and resuspended at a final concentration of 10⁷ cells/ml in Opti-MEM. 400 ml of cells (4×10⁶ cells) plus 10 μg of in vitro transcribed Jc1 or Con1/C3-neo RNA were added to 0.4 cm electroporation cuvettes (BioRad). Electroporation was performed using a Gene Pulser (BioRad) using the following parameters: 0.27 kV, 100 Ohms and 950 μF. The cuvettes were incubated at RT for 10 minutes to allow the cells to recuperate before transferring the cells into one T162 flask containing c-DMEM. Cells were trypsinized and split when cultures reached 80-90% of confluency as required. Supernatants were harvested starting at 3 days post transfection, clarified and infectious viral titers were measured using the TCID₅₀ calculation method as described previously. See Lindenbach, B. D., et al., Science 309:623-6 (2005). Supernatants were aliquoted and stored at −70° C.

Generation of HCV Pseudoparticles (HCVpp)

Plasmids: Plasmids expressing E1 and E2 glycoproteins from HCV genotypes 1a (H77), 1b (Con1) and 2a (J6) were generated as previously described (Hsu, M. et al, Proc Natl Acad Sci USA 100:7271-6 (2003)) with some modifications. Briefly, the region encoding E1 and E2 (and containing the signal peptide from the C-terminus of HCV core) was cloned into the pRK mammalian expression vector to generate the expression plasmids, pRK-H77, pRK-Con1 and pRK-J6, respectively. The Δ8.9 packaging plasmid was originally acquired by Genentech from Greg Hannon (Cold Spring Harbor Labs)/David Baltimore (Cal Tech). See Zufferey et al., Nature Biotechnology 15:871-875 (1997). The FCMV-Luc-IRES-dsRED plasmid is a modified pFUGW plasmid, which was obtained by Genentech from Greg Hannon at Cold Spring Harbor Labs, and encodes firefly luciferase and DsRed driven by the HCMV promoter and IRES element, respectively.

HCVpp were produced in HEK 293T cells as described previously (Bartosch, B. et al., J Exp Med 197:633-42 (2003)) with some modifications. Briefly, 2.5×10⁶ 293T cells were seeded the day before in 10-cm plates. The following day, the cells were co-transfected with the FCMV-Luc-IRES-DsRed plasmid (5 μg), Δ8.9 transfer vector (10 μg) and either the pRK-H77, pRK-Con1 or pRK-J6 plasmids (1 μg) using Lipofectamine 2000 (Invitrogen), as per manufacturer's recommendations. Six hours post-transfection the OptiMEM medium (Invitrogen, Gibco) was replaced with c-DMEM. Two days post transfection, supernatants were harvested, clarified and further purified by ultracentrifugation (3000 rpm for 5 minutes) and used in infectivity assays. 5×10³ Huh-7.5 cells were seeded in white walled 96-well plates (Costar). The following day, cells were transduced with appropriate dilution of HCVpp. Seventy-two hours post-infection, cells were lysed in 1× lysis buffer and luciferase activity was measured using the Luciferase Assay System (Promega), as per manufacturer's recommendations.

ELISA assay to determine antibody binding to HCV E2

Preparation of E2 lysates: 293T cells were transiently transfected with 10 μg pRK-H77, pRK-Con1 or pRK-J6 plasmids using Lipofectamine 2000, as per manufacturer's recommendations. Forty-eight hours post transfection, cells were washed with PBS and then lysed in 1 μL lysis buffer (20 mM Tris-HCl, pH 7.4; 150 mM NaCl; 1 mM EDTA; 0.5% NP-40; mM iodoacetamide). The lysate was incubated with shaking at 4° C. for 20 minutes and centrifuged for 5 minutes. The clarified supernatant as then used to coat the ELISA plates.

HCV E2 ELISA: ELISA assay was performed as previously described. See Owsianka, A. et al., J Virol 79:11095-104 (2005). Briefly, 96-well Immulon 2 plates were coated with 0.25 μg/well Galanthus nivalis lectin (GNA, Sigma) in 100 μL PBS and incubated at RT overnight. The following day, plates were washed 3× with PBS containing 0.02% Tween-20 (PBST), coated with cell lysate diluted in PBST and incubated at RT for 2 hours. Dilutions of chronic HCV-infected patient sera were diluted in 2% skimmed milk powder/PBST and incubated for 1 hour at RT. After 3× washes with PBST, 100 μL/well of anti-human HRP conjugated secondary antibody was added at a dilution of 1:1000 in PBST and incubated for 1 hour at RT. Wells were washed 6× with PBST and wells were incubated with 100 μL TMB substrate in the dark at RT for 30 minutes. Reactions were stopped by adding 50 μL/well of 0.5M H₂SO₄ and A₄₅₀ was measured using a Synergy 2 plate reader (BioTek Instruments).

HCVcc Infection Assays

For infections in 96-well plates, 5×10³ Huh-7.5 cells/well were plated. The following day, the cells were infected with Jc1 or Con1/C3-neo HCVcc at a multiplicity of infection (MOI)=0.3. To identify antibodies that neutralize HCVcc, antibodies were diluted to 150 μg/ml in c-DMEM and seven 3-fold dilutions of the antibody were made in a separate 96-well plate. HCVcc and antibody dilutions were combined and pre-incubated for 1 hour at 37° C. prior to inoculating naïve Huh-7.5 cells. Total RNA was harvested 3 days post infection and HCV RNA replication (measured as a ratio of HCV/GAPDH cDNA) was determined using RT-qPCR, as described below.

Quantitation of HCV Infection

HCVcc RNA replication: For experiments performed in 96-well plates, total RNA was extracted using the SV96 Total RNA Isolation System (Promega), according to manufacturer's instructions. RNA from each well was eluted into 100 μL of RNase-free water and 4 μL of RNA was reverse transcribed using the Taqman Reverse Transcription Reagent Kit (Applied Biosystems). RT-qPCR was performed using 5 μL of cDNA in a 25 μL reaction using TaqMan Universal PCR Master Mix (Applied Biosystems). In all reactions, expression of the housekeeping gene glyceraldehyde-3-phosphate dehydrogenase (GAPDH) was determined as an internal endogenous control for amplification efficiency and normalization. The primers and probes for HCV and GAPDH are as follows: GT1b sense primer, 5′-CTGCGGAACCGGTGAGTACA-3′ (SEQ ID NO:204); GT1b anti-sense primer, 5′-TGCACGGTCTACGAGACCTCC-3′ (SEQ ID NO:205); GT1b probe, 6FAM-ACCCGGTCGTCCTGGCAATTCC-MGBNFQ (SEQ ID NO:206); GT2a sense primer, 5′-CTTCACGCAGAAAGCGCCTA (SEQ ID NO:207); GT2a anti-sense primer, 5′-CAAGCACCCTATCAGGCAGT-3′ (SEQ ID NO:208); GT2a probe, 6FAM-TATGAGTGTCGTACAGCCTC-MGBNFQ (SEQ ID NO:209); GAPDH sense primer, 5′-GAAGGTGAAGGTCGGAGTC-3′ (SEQ ID NO:210); GAPDH anti-sense primer, 5′-GAAGATGGTGATGGGATTTC-3′ (SEQ ID NO:211); GAPDH probe, VIC-ATGACCCCTTCA TTGACCTC-MGBNFQ (SEQ ID NO:212). Fluorescence was monitored using a 7500 HT real-time PCR machine (Applied Biosystems, CA).

Titration of infectious HCVcc: Infectious HCVcc present in the supernatants of infected cells was measured as described previously. See Lindenbach, B. D. et al., Science 309:623-6 (2005). Briefly, titrations were performed by seeding 5×10³ Huh-7.5 cells in poly-L-lysine coated 96-well plates. The following day, cells were inoculated with 10-fold dilutions of supernatants in a final volume of 100 μL. Three days later, cells were fixed with 4% paraformaldehyde in PBS and immunostained as described previously (Kapadia, S. B. et al., J Virol 81:374-83 (2007)) with an anti-HCV core antibody, C₇₋₅₀ (Abcam). Titers were calculated according to the method of Reed and Muench as described previously. See Lindenbach, B. D. et al., Science 309:623-6 (2005).

Effect of Sera from Chronically HCV-Infected Patients on RH-C/RK2b-Mediated Neutralization

To determine whether sera from chronic HCV-infected patients antagonized RH-C/RK2b-mediated neutralization of HCV infection in vitro, neutralization assays were performed using the HCVpp system. HCVpp was incubated with different concentrations of RH-C/RK2b for 1 h at 37° C. in the presence of either 10% fetal bovine serum (FBS), 10% normal human serum (NHS), or 10% of sera from chronic HCV-infected patients (CHCHS-1, 2 and 3). Huh −7.5 cells seeded in 96-well plates were inoculated with the HCVpp:antibody mixture. Four hours post-transduction, the medium was replaced with c-DMEM containing 10% FBS plus supplements for the remainder of the assay. Three days later, cells were lysed and luciferase activity was measured as described above. Levels of anti-HCV E1/E2 antibodies were determined using ELISA as described above.

### Example 2A

### Neutralization of HCVcc and HCVpp by RH-C/RK2b

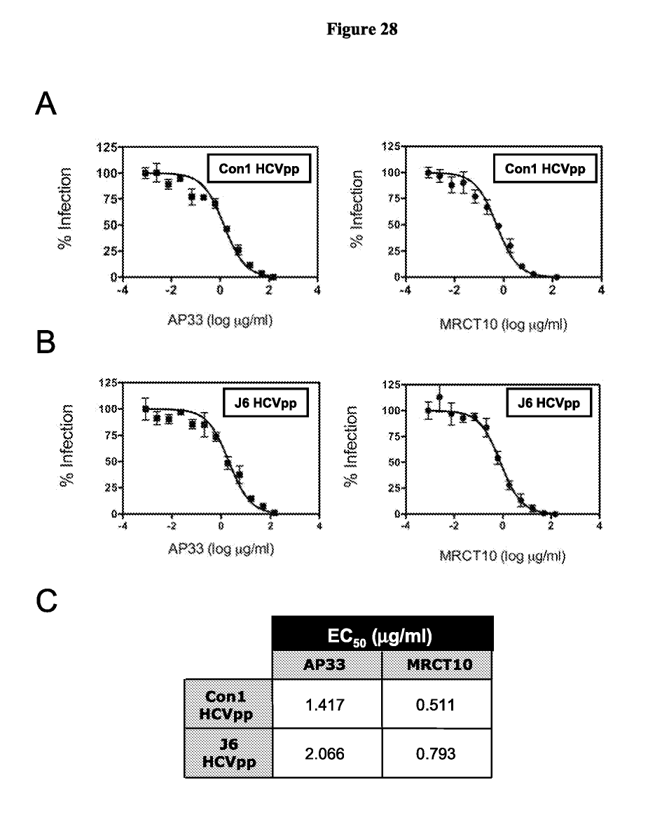

To determine whether RH-C/RK2b inhibits HCV entry and infection, a neutralization assay was performed in Huh-7.5 cells using both HCVpp and HCVcc. AP33 was used as a control. To identify the specific inhibition of HCV entry by RH-C/RK2b, HCVpp containing E1E2 sequences from GT1b (Con1) or GT2a (J6) were incubated in the presence of AP33 or RH-C/RK2b. RH-C/RK2b inhibited Con1 and J6 HCVpp entry equivalently (EC₅₀=0.511 μg/mL and 0.793 μg/mL for Con1 and J6 HCVpp, respectively). See FIGS. 28A-C. In addition, RH-C/RK2b neutralization of both HCVpp genotypes was comparable to that seen with AP33 (EC₅₀=1.417 μg/mL and 2.066 μg/mL for Con1 and J6 HCVpp, respectively (FIGS. 28A-C).

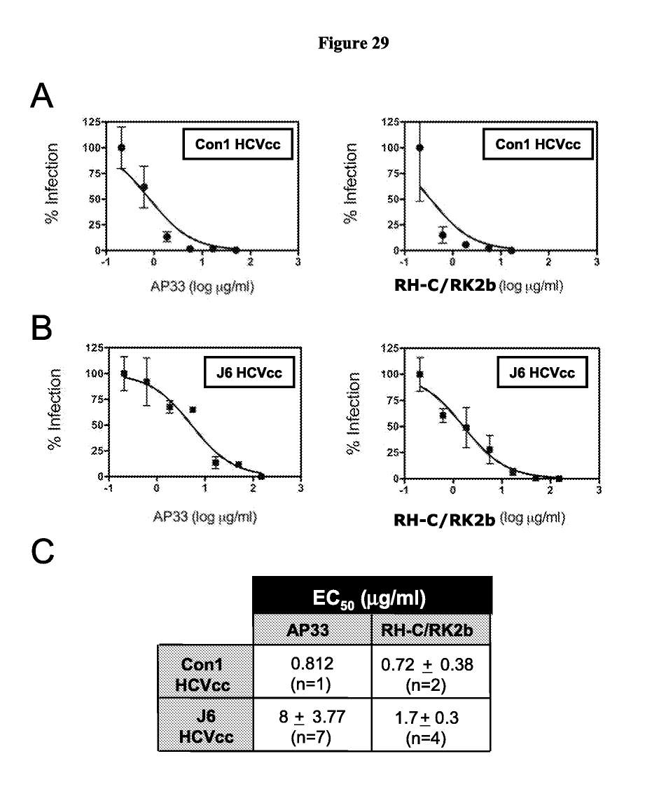

To determine if RH-C/RK2b neutralized the infectious cell culture virus (HCVcc), similar neutralization assays were performed with AP33 and RH-C/RK2b. AP33 inhibited both Con1 and J6 HCVcc to levels comparable to that previously described for HCVpp containing E1E2 sequences from multiple genotypes (Owsianka, A. et al., J Virol 79:11095-104 (2005)). While RH-C/RK2b inhibited Con1 HCVcc infection to levels comparable to AP33 (FIGS. 29A and C), RH-C/RK2b inhibited J6 HCVcc at least ˜4.7-fold better than AP33 (FIGS. 29B-C).

### Example 2B

### Affinity Measurements of Anti-HCV E2 Antibodies to E2

In further experiments, the affinity of AP33 and RH-C/RK2b to soluble E2 (sE2) was determined by BIAcore assays. Both AP33 and RH-C/RK2b bound sE2 with similar affinities (−5-8 nM for AP33 and ˜3.8 nM for RH-C/RK2b). In comparison, the Fab fragments of each antibody bound sE2 with an affinity of ˜0.50 nM. In addition to binding sE2 protein, binding of AP33 and RH-C/RK2b to E1E2 heterodimers expressed on the surface of 293T cells was determined. Since it is known that 293T cells transfected with plasmids encoding E1E2 express functional E1E2 heterodimers on their cell surface, scatchard analysis was performed to determine affinities of RH-C/RK2b and RH-C/RK2b Fab. Affinities of RH-C/RK2b and RH-C/RK2b Fab to cell surface expressed E2 (−5 and ˜50 nM, respectively) were comparable to that seen with sE2 in the BIAcore assay described above. See Table 7.

TABLE 7
Antibody Affinity (nM)

| Antibody | sE2 | E1E2 Scatchard |
|---|---|---|
| AP33 | 5-8 | |
| AP33 Fab | 50 | |
| RH-C/RK2b | 3.8 ± 0.6 | 5 |
| RH-C/RK2b Fab | | 50 |

### Example 2C

### Sera from Chronic HCV-Infected Patients do not Antagonize RH-C/RK2b-Mediated Neutralization

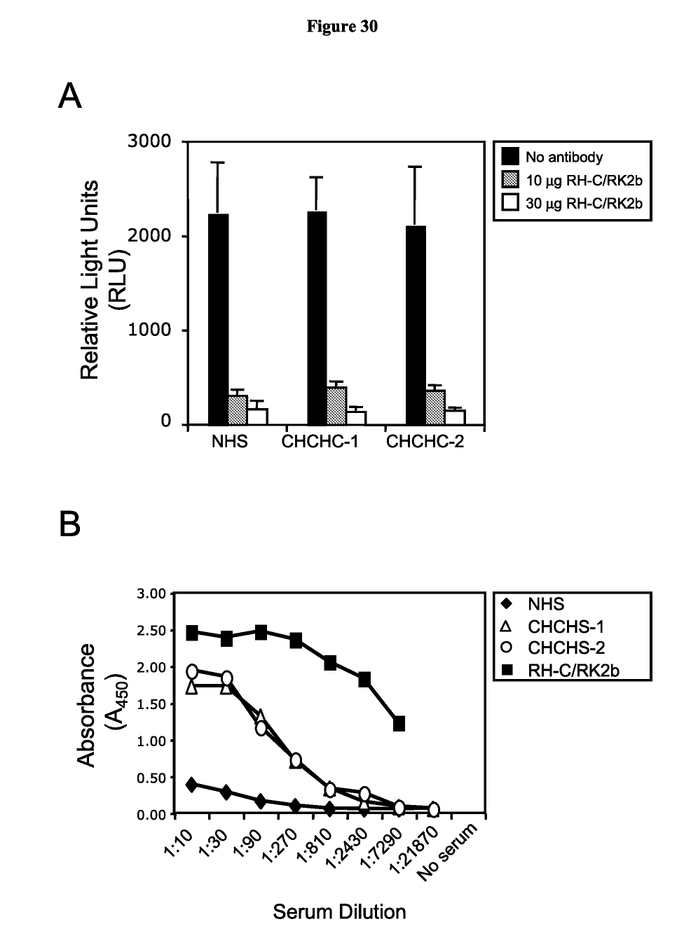

In order to determine whether chronic patient sera, which contain anti-HCV antibodies, can antagonize the neutralizing ability of RH-C/RK2b, a neutralization assay was performed using Con1 HCVpp in the presence of 10% normal human serum (NHS) or sera from chronic HCV-infected patients (CHCHS-1 and -2). RH-C/RK2b inhibited HCV infection to comparable levels irrespective of the source of human serum (FIG. 30A). To determine whether these chronic HCV-infected patient sera contained antibodies against genotype 1b, an ELISA assay was performed using lysates from GT1b (Con1) E1E2-transfected 293T cells. 3-fold dilutions of RH-C/RK2b starting at an initial concentration of 10 μg/mL were used as controls. While no binding to E2 was detected with NHS, dose-dependent binding was detected with both chronic HCV-infected patient sera, suggesting that they contained Con1 HCV E1E2-reactive antibodies. See FIG. 30B. These results suggest that while anti-HCV antibodies do exist in patient sera, they do not interfere with the ability of RH-C/RK2b to neutralize HCV in vitro.

### Example 3

### Synergistic Inhibition of HCVcc Infection Between IFN-a and RH-C/RK2B In Vitro

To test whether the addition of RH-C/RK2b enhanced the antiviral effect of IFN-a in vitro, Huh-7.5 cells were infected with Jc1 or Con1/C3-neo HCVcc in the presence of RH-C/RK2b alone, IFN-a alone or in combination of both RH-C/RK2b plus IFN-a.

Huh-7.5 cells were differentiated by growing them in c-DMEM containing 1% dimethyl sulfoxide (DMSO), as described previously. See Sainz, B., Jr., and F. V. Chisari., J Virol 80:10253-7 (2006). This would prevent virus spread due to cell proliferation and would specifically measure HCV spread. Briefly, Huh-7.5 cells were seeded in Biocoat plates (Beckton Dickinson) and grown to 90% confluency before switching to 1% DMSO-containing c-DMEM for two weeks. Medium was changed every two days. The cells were infected with either Jc1 or Con1/C3-neo alone or in the presence of RH-C/RK2b alone, IFN-a alone or the combination of both RH-C/RK2b+IFN-a+. a-interferon was purchased from (PBL Biomedical Laboratories). Total RNA was harvested every four days and HCV RNA replication was measured as described above in Example 2.

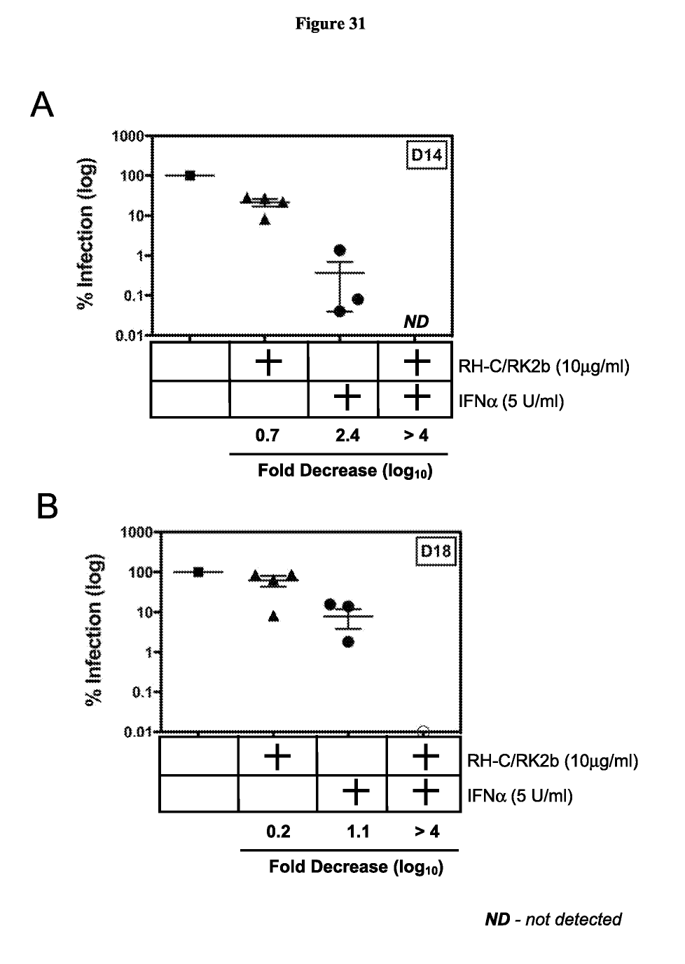

HCV RNA was measured at day 14 or 18 post infection to analyze infection. At day 14 post infection, while RH-C/RK2b and IFN-a alone decreased infection by ˜5-fold and ˜250-fold, respectively, there was at least a 1000-fold decrease in HCV RNA replication in the presence of RH-C/RK2b plus IFN-a (FIG. 31A). Similar synergistic effects were identified on day 18 post infection (FIG. 31B).

### Example 4

### Synergistic Inhibition of HCVcc Infection Between IFN-a and RH-H/RK2B and RHb-H/RK2b In Vitro

To test whether the addition of RH-H/RK2b or RHb-H/RK2b enhance the antiviral effect of IFN-a in vitro, Huh-7.5 cells are infected with Jc1 or Con1/C3-neo HCVcc in the presence of RH-H/RK2b alone, RHb-H/RK2b alone, IFN-a alone or in combination of both RH-H/RK2b or RHb-H/RK2b plus IFN-a.

Huh-7.5 cells are differentiated by growing them in c-DMEM containing 1% dimethyl sulfoxide (DMSO), as described previously. See Sainz, B., Jr., and F. V. Chisari., J Virol 80:10253-7 (2006). This prevents virus spreading due to cell proliferation and specifically measures HCV spread. Briefly, Huh-7.5 cells are seeded in Biocoat plates (Beckton Dickinson) and are grown to 90% confluency before switching to 1% DMSO-containing c-DMEM for two weeks. Medium is changed every two days. The cells are infected with either Jc1 or Con1/C3-neo alone or in the presence of RH-H/RK2b alone, RHb-H/RK2b alone, IFN-a alone or in combination of both RH-H/RK2b or RHb-H/RK2b plus IFN-a. a-interferon is purchased from (PBL Biomedical Laboratories). Total RNA is harvested every four days and HCV RNA replication is measured as described above in Example 2.

1. A method of treating or preventing a hepatitis C virus (HCV) infection in a subject, comprising administering to the individual: a) an effective amount of a composition comprising an anti-HCV antibody that binds hepatitis E2 protein; and b) an effective amount of α-interferon.

2. The method of claim 1, wherein the anti-HCV antibody is a monoclonal antibody.

3. The method of claim 2, wherein the monoclonal antibody comprises (a) a light chain variable domain comprising (i) CDR-L1 comprising sequence RASESVDGYGNSFLH (SEQ ID NO:41); (ii) CDR-L2 comprising sequence LASNLNS (SEQ ID NO:42); and (iii) CDR-L3 comprising sequence QQNNVDPWT (SEQ ID NO:43) and (b) a heavy chain variable domain comprising (i) CDR-H1 comprising sequence GDSITSGYWN (SEQ ID NO:44); (ii) CDR-H2 comprising sequence YISYSGSTY (SEQ ID NO:45); and (iii) CDR-H3 comprising sequence ITTTTYAMDY (SEQ ID NO:46).

4. The method of claim 2, wherein the monoclonal antibody comprises (a) a light chain variable domain comprising (i) CDR-L1 comprising sequence RASESVDGYGNSFLH (SEQ ID NO:41); (ii) CDR-L2 comprising sequence LASNLNS (SEQ ID NO:42); and (iii) CDR-L3 comprising sequence QQNNVDPWT (SEQ ID NO:43) and (b) a heavy chain variable domain comprising (i) CDR-H1 comprising sequence SGYWN (SEQ ID NO:47); (ii) CDR-H2 comprising sequence YISYSGSTYYNLSLRS (SEQ ID NO:48); and (iii) CDR-H3 comprising sequence ITTTTYAMDY (SEQ ID NO:46).

5. The method of claim 2, wherein the monoclonal antibody is a humanized antibody.

6. The method of claim 5, wherein the humanized antibody comprises (a) a light chain variable domain comprising (i) CDR-L1 comprising sequence RASESVDGYGNSFLH (SEQ ID NO:41); (ii) CDR-L2 comprising sequence LASNLNS (SEQ ID NO:42); and (iii) CDR-L3 pa-1470224 comprising sequence QQNNVDPWT (SEQ ID NO:43) and (b) a heavy chain variable domain comprising (i) CDR-H1 comprising sequence GDSITSGYWN (SEQ ID NO:44); (ii) CDR-H2 comprising sequence YISYSGSTY (SEQ ID NO:45); and (iii) CDR-H3 comprising sequence ITTTTYAMDY (SEQ ID NO:46).

7. The method of claim 5, wherein the humanized antibody comprises (a) a light chain variable domain comprising (i) CDR-L1 comprising sequence RASESVDGYGNSFLH (SEQ ID NO:41); (ii) CDR-L2 comprising sequence LASNLNS (SEQ ID NO:42); and (iii) CDR-L3 comprising sequence QQNNVDPWT (SEQ ID NO:43) and (b) a heavy chain variable domain comprising (i) CDR-H1 comprising sequence SGYWN (SEQ ID NO:47); (ii) CDR-H2 comprising sequence YISYSGSTYYNLSLRS (SEQ ID NO:48); and (iii) CDR-H3 comprising sequence ITTTTYAMDY (SEQ ID NO:46).

8. The method of claim 5, wherein the humanized antibody comprises a variable heavy chain domain selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18 and a variable light chain domain selected from the group consisting of SEQ ID NO:6, SEQ ID NO:7, SEQ ID NO:19, and SEQ ID NO:20.

9. The method of claim 5, wherein the humanized antibody is an antigen binding fragment.

10. The method of claim 9, wherein the antigen binding fragment is selected from the group consisting of a Fab fragment, a Fab′ fragment, a F(ab′)₂ fragment, a scFv, a Fv, and a diabody.

11. The method of claim 1, wherein the α-interferon is selected from a group consisting of IFN-α1, IFN-α2, IFN-α4, IFN-α5, IFN-α6, IFN-α7, IFN-α8, IFN-α10, IFN-α13, IFN-α14, IFN-α16, IFN-α17, and IFN-α21.

12. The method of claim 11, wherein the α-interferon is IFN-a2.

13. The method of claim 12, wherein the IFN-α2 is selected from the group consisting of IFN-α2a, IFN-α2b, or IFN-α2c.

14. The method of claim 13, wherein the IFN-α2 is pegylated.

15. The method of claim 1, wherein the anti-HCV antibody is administered simultaneously, concurrently, rotationally, intermittently, or sequentially with α-interferon.

16. The method of claim 1, wherein the hepatitis C virus infection is an acute hepatitis C virus infection.

17. The method of claim 1, wherein the hepatitis C virus infection is a chronic hepatitis C virus infection.

18. The method of claim 1, wherein treating the hepatitis C virus infection comprises reducing viral load.

19. The method of claim 1, wherein treating the hepatitis C virus infection comprises reducing viral titer.
